SEQUENCE LISTING (110 sequences)

SEQ ID NO: 1 - DNA, 18 bp, Artificial
  PCR primer and/or probe
ccaaccgcgagaagatga

SEQ ID NO: 2 - DNA, 18 bp, Artificial
  PCR primer and/or probe
tccatcacgatgccagtg

SEQ ID NO: 3 - DNA, 18 bp, Artificial
  PCR primer and/or probe
cttcgtctccgccatcag

SEQ ID NO: 4 - DNA, 18 bp, Artificial
  PCR primer and/or probe
ttgccaatctggacacca

SEQ ID NO: 5 - DNA, 20 bp, Artificial
  PCR primer and/or probe
ccacggagatggtagagtgc

SEQ ID NO: 6 - DNA, 20 bp, Artificial
  PCR primer and/or probe
cgatgaaaatgttcccttgc

SEQ ID NO: 7 - DNA, 18 bp, Artificial
  PCR primer and/or probe
ggtctgcggggctacttt

SEQ ID NO: 8 - DNA, 23 bp, Artificial
  PCR primer and/or probe
tgcctcacaaacaagtacatcag

SEQ ID NO: 9 - DNA, 23 bp, Artificial
  PCR primer and/or probe
tctcagatggaggaatttttgat

SEQ ID NO: 10 - DNA, 17 bp, Artificial
  PCR primer and/or probe
agccaggtcgtcaggag

SEQ ID NO: 11 - DNA, 26 bp, Artificial
  PCR primer and/or probe
aatcgtgagtctccactataagacag

SEQ ID NO: 12 - DNA, 22 bp, Artificial
  PCR primer and/or probe
gcctgtaaagagacaggaaacc

SEQ ID NO: 13 - DNA, 21 bp, Artificial
  PCR primer and/or probe
gatcttcaacttccggaggac

SEQ ID NO: 14 - DNA, 18 bp, Artificial
  PCR primer and/or probe
actgggaggcgaagaagg

SEQ ID NO: 15 - DNA, 19 bp, Artificial
  PCR primer and/or probe
catgcctgtcctgggattt

SEQ ID NO: 16 - DNA, 20 bp, Artificial
  PCR primer and/or probe
agaatcaatatggcggaagc

SEQ ID NO: 17 - DNA, 18 bp, Artificial
  PCR primer and/or probe
ctatgcgcctgcagaggt

SEQ ID NO: 18 - DNA, 25 bp, Artificial
  PCR primer and/or probe
acctgctcctcattattgtaaacat

SEQ ID NO: 19 - DNA, 20 bp, Artificial
  PCR primer and/or probe
cattggggtgtcaaacttca

SEQ ID NO: 20 - DNA, 20 bp, Artificial
  PCR primer and/or probe
ccggttgaaatacggatgac

SEQ ID NO: 21 - DNA, 20 bp, Artificial
  PCR primer and/or probe
gacgggtgatttctgcattt

SEQ ID NO: 22 - DNA, 24 bp, Artificial
  PCR primer and/or probe
ctctcttgaagttttcaattgtgc

SEQ ID NO: 23 - DNA, 18 bp, Artificial
  PCR primer and/or probe
gacgtcctgaggctggtg

SEQ ID NO: 24 - DNA, 22 bp, Artificial
  PCR primer and/or probe
ggtgcaggaaatgtaattgttg

SEQ ID NO: 25 - DNA, 21 bp, Artificial
  PCR primer and/or probe
cacacttctctccgcgtacat

SEQ ID NO: 26 - DNA, 21 bp, Artificial
  PCR primer and/or probe
gttgtagcctccttcccaagt

SEQ ID NO: 27 - DNA, 18 bp, Artificial
  PCR primer and/or probe
catttggcccatggattc

SEQ ID NO: 28 - DNA, 20 bp, Artificial
  PCR primer and/or probe
ccaatggtcacatggaattg

SEQ ID NO: 29 - DNA, 18 bp, Artificial
  PCR primer and/or probe
cccttccatgcgtccata

SEQ ID NO: 30 - DNA, 22 bp, Artificial
  PCR primer and/or probe
gattgtcagcacaaactgaagc

SEQ ID NO: 31 - DNA, 19 bp, Artificial
  PCR primer and/or probe
ttctgctggacaccctgaa

SEQ ID NO: 32 - DNA, 20 bp, Artificial
  PCR primer and/or probe
ttcagattagccccgatgtc

SEQ ID NO: 33 - DNA, 19 bp, Artificial
  PCR primer and/or probe
ttcccgtgtaaccagttcg

SEQ ID NO: 34 - DNA, 20 bp, Artificial
  PCR primer and/or probe
cggcgaactctttgatctct

SEQ ID NO: 35 - DNA, 19 bp, Artificial
  PCR primer and/or probe
ctgctctcacggtccagtc

SEQ ID NO: 36 - DNA, 27 bp, Artificial
  PCR primer and/or probe
ttttctattgtaaggtcctttgtatcc

SEQ ID NO: 37 - DNA, 21 bp, Artificial
  PCR primer and/or probe
tgctgcttaacctttcagctt

SEQ ID NO: 38 - DNA, 20 bp, Artificial
  PCR primer and/or probe
atccttctgggcctacaggt

SEQ ID NO: 39 - DNA, 20 bp, Artificial
  PCR primer and/or probe
accatgaaggacgaggtagc

SEQ ID NO: 40 - DNA, 20 bp, Artificial
  PCR primer and/or probe
tgcagggagaagtaggcttg

SEQ ID NO: 41 - DNA, 20 bp, Artificial
  PCR primer and/or probe
ccaacttccagcaggacaag

SEQ ID NO: 42 - DNA, 20 bp, Artificial
  PCR primer and/or probe
ccacagacttgcacatggac

SEQ ID NO: 43 - DNA, 18 bp, Artificial
  PCR primer and/or probe
agagatgcctgcccacag

SEQ ID NO: 44 - DNA, 19 bp, Artificial
  PCR primer and/or probe
tggccaccacgtacatctt

SEQ ID NO: 45 - DNA, 20 bp, Artificial
  PCR primer and/or probe
gcagggctgagatcaagttc

SEQ ID NO: 46 - DNA, 25 bp, Artificial
  PCR primer and/or probe
gacagaggagtcatttagttgttgc

SEQ ID NO: 47 - DNA, 22 bp, Artificial
  PCR primer and/or probe
ttaacaaaagaaagggcaaagc

SEQ ID NO: 48 - DNA, 26 bp, Artificial
  PCR primer and/or probe
tcagagaaacgatcaaaattagacat

SEQ ID NO: 49 - DNA, 21 bp, Artificial
  PCR primer and/or probe
gatttttgatgtgcagcttcc

SEQ ID NO: 50 - DNA, 20 bp, Artificial
  PCR primer and/or probe
gtgagtgcctggagctctct

SEQ ID NO: 51 - DNA, 18 bp, Artificial
  PCR primer and/or probe
atgatggggcagcaagtg

SEQ ID NO: 52 - DNA, 19 bp, Artificial
  PCR primer and/or probe
cccatcagaaatggagtgc

SEQ ID NO: 53 - DNA, 20 bp, Artificial
  PCR primer and/or probe
tccatgttggtggactatgg

SEQ ID NO: 54 - DNA, 19 bp, Artificial
  PCR primer and/or probe
gtggtggtccatgttcctg

SEQ ID NO: 55 - DNA, 18 bp, Artificial
  PCR primer and/or probe
gatccccagggctcaaac

SEQ ID NO: 56 - DNA, 26 bp, Artificial
  PCR primer and/or probe
tcaccgtaaatatgatttaagtccac

SEQ ID NO: 57 - DNA, 20 bp, Artificial
  PCR primer and/or probe
ctgccctatctggacatggt

SEQ ID NO: 58 - DNA, 18 bp, Artificial
  PCR primer and/or probe
tgcctcccgtgtgaatct

SEQ ID NO: 59 - DNA, 20 bp, Artificial
  PCR primer and/or probe
tgatcctcacagctgttgct

SEQ ID NO: 60 - DNA, 23 bp, Artificial
  PCR primer and/or probe
tccattctcttgtcgaagtttct

SEQ ID NO: 61 - DNA, 18 bp, Artificial
  PCR primer and/or probe
agtctctgccgcccttct

SEQ ID NO: 62 - DNA, 19 bp, Artificial
  PCR primer and/or probe
gtgactggggcattgattg

SEQ ID NO: 63 - DNA, 20 bp, Artificial
  PCR primer and/or probe
taggctgagcaacatgaagg

SEQ ID NO: 64 - DNA, 19 bp, Artificial
  PCR primer and/or probe
gctgggtggcagtaggaat

SEQ ID NO: 65 - DNA, 22 bp, Artificial
  PCR primer and/or probe
gctgaacagtgacaaatccaac

SEQ ID NO: 66 - DNA, 20 bp, Artificial
  PCR primer and/or probe
cttcaggaacagccaccagt

SEQ ID NO: 67 - DNA, 21 bp, Artificial
  PCR primer and/or probe
gattggatcaggaccatgtga

SEQ ID NO: 68 - DNA, 23 bp, Artificial
  PCR primer and/or probe
ccattctcattttatgttgctca

SEQ ID NO: 69 - DNA, 21 bp, Artificial
  PCR primer and/or probe
ggttgagtttaagccaatcca

SEQ ID NO: 70 - DNA, 20 bp, Artificial
  PCR primer and/or probe
tgctgacctaggcttgatga

SEQ ID NO: 71 - DNA, 20 bp, Artificial
  PCR primer and/or probe
ctgtcctgcgtgttgaaaga

SEQ ID NO: 72 - DNA, 23 bp, Artificial
  PCR primer and/or probe
ttgggtaatttttgggatctaca

SEQ ID NO: 73 - DNA, 21 bp, Artificial
  PCR primer and/or probe
gcccagctatgaactccttct

SEQ ID NO: 74 - DNA, 18 bp, Artificial
  PCR primer and/or probe
gaaggcagcaggcaacac

SEQ ID NO: 75 - DNA, 20 bp, Artificial
  PCR primer and/or probe
agacagcagagcacacaagc

SEQ ID NO: 76 - DNA, 18 bp, Artificial
  PCR primer and/or probe
atggttccttccggtggt

SEQ ID NO: 77 - DNA, 20 bp, Artificial
  PCR primer and/or probe
gatgccttcagcagagtgaa

SEQ ID NO: 78 - DNA, 21 bp, Artificial
  PCR primer and/or probe
gcaacccaggtaacccttaaa

SEQ ID NO: 79 - DNA, 27 bp, Artificial
  PCR primer and/or probe
caaaacatatttctttgtagaggacaa

SEQ ID NO: 80 - DNA, 21 bp, Artificial
  PCR primer and/or probe
ttcagctatttgcttgggaaa

SEQ ID NO: 81 - DNA, 20 bp, Artificial
  PCR primer and/or probe
gcagtcagatccacagcatc

SEQ ID NO: 82 - DNA, 22 bp, Artificial
  PCR primer and/or probe
tccaaagaatcctcactcccta

SEQ ID NO: 83 - DNA, 20 bp, Artificial
  PCR primer and/or probe
ggattttggaaaccagcaga

SEQ ID NO: 84 - DNA, 20 bp, Artificial
  PCR primer and/or probe
ccgtctctctcttcctcgac

SEQ ID NO: 85 - DNA, 22 bp, Artificial
  PCR primer and/or probe
tcactgtcttgtacccttgtgc

SEQ ID NO: 86 - DNA, 20 bp, Artificial
  PCR primer and/or probe
ggcgtttggagtggtagaaa

SEQ ID NO: 87 - DNA, 19 bp, Artificial
  PCR primer and/or probe
ttggatgctcttggggttc

SEQ ID NO: 88 - DNA, 20 bp, Artificial
  PCR primer and/or probe
aagcagctggagtggttgtt

SEQ ID NO: 89 - DNA, 20 bp, Artificial
  PCR primer and/or probe
ctaaggccaaccgtgaaaag

SEQ ID NO: 90 - DNA, 20 bp, Artificial
  PCR primer and/or probe
accagaggcatacagggaca

SEQ ID NO: 91 - DNA, 18 bp, Artificial
  PCR primer and/or probe
ctggaacccacggtcatc

SEQ ID NO: 92 - DNA, 21 bp, Artificial
  PCR primer and/or probe
gtggccacgagcatagttatt

SEQ ID NO: 93 - DNA, 20 bp, Artificial
  PCR primer and/or probe
aggcacggcaaaaacagtat

SEQ ID NO: 94 - DNA, 20 bp, Artificial
  PCR primer and/or probe
tgtggcatttggcatcaata

SEQ ID NO: 95 - DNA, 20 bp, Artificial
  PCR primer and/or probe
ctcttctggctaccgtcacc

SEQ ID NO: 96 - DNA, 19 bp, Artificial
  PCR primer and/or probe
aagcccttcgtgcagagat

SEQ ID NO: 97 - DNA, 21 bp, Artificial
  PCR primer and/or probe
ggctcctggacactacaccta

SEQ ID NO: 98 - DNA, 20 bp, Artificial
  PCR primer and/or probe
atagttggcctccaccactg

SEQ ID NO: 99 - DNA, 20 bp, Artificial
  PCR primer and/or probe
agcacactgctggtcatcaa

SEQ ID NO: 100 - DNA, 18 bp, Artificial
  PCR primer and/or probe
cagcctcatctggcctgt

SEQ ID NO: 101 - DNA, 21 bp, Artificial
  PCR primer and/or probe
gggagtctggaacattgtgaa

SEQ ID NO: 102 - DNA, 24 bp, Artificial
  PCR primer and/or probe
gtgcacattgtaagtaggtggact

SEQ ID NO: 103 - DNA, 18 bp, Artificial
  PCR primer and/or probe
ctcctggtgagcgaggac

SEQ ID NO: 104 - DNA, 19 bp, Artificial
  PCR primer and/or probe
gaccaggttgcccatcact

SEQ ID NO: 105 - DNA, 18 bp, Artificial
  PCR primer and/or probe
tggcacaaaagggacgag

SEQ ID NO: 106 - DNA, 19 bp, Artificial
  PCR primer and/or probe
ggccaggaataccaggaag

SEQ ID NO: 107 - DNA, 20 bp, Artificial
  PCR primer and/or probe
tccacagcgatatccagaca

SEQ ID NO: 108 - DNA, 20 bp, Artificial
  PCR primer and/or probe
ggacatcaccaggattggac

SEQ ID NO: 109 - DNA, 20 bp, Artificial
  PCR primer and/or probe
aactctttcggtcgtacccc

SEQ ID NO: 110 - DNA, 19 bp, Artificial
  PCR primer and/or probe
tcctcgcagttcgaatctg

(19) United States
(12) Patent Application Publication
(10) Pub. No.: US 2020/0371122 A1
(43) Pub. Date: Nov. 26, 2020

(54) BIOMARKER FOR SENESCENT CELLS

(71) Applicant: Buck Institute for Research on Aging, Novato, CA (US)

(72) Inventors: Christopher D. Wiley, Sonoma, CA (US); Judith Campisi, Berkeley, CA (US); Arvind Ramanathan, Novato, CA (US)

(21) Appl. No.: 16/652,963

(22) PCT Filed: Sep. 19, 2018
(86) PCT No.: PCT/US2018/051803

§ 371 Date: Apr. 1, 2020

Related U.S. Application Data
(60) Provisional application No. 62/569,437, filed on Oct. 6, 2017.

(51) Int. Cl. G01N 33/88; A61K 31/496

(52) U.S. Cl. CPC G01N 33/88; A61K 31/496; G01N 2800/52; G01N 2800/7042; G01N 2800/50

(57) ABSTRACT

Various biological markers that function as indicators of the level of senescent cells in an organism are provided. In certain embodiments, the markers described herein (e.g., eicosanoids) can provide effective indicators of the presence and/or quantity of senescent cells in a subject (e.g., in a human or non-human mammal) and methods of identifying elevated levels of senescent cells in a mammal, and methods for determining the efficacy of senolytic agents, are provided.

### CROSS-REFERENCE TO RELATED APPLICATIONS

This application claims benefit of and priority to U.S. Ser. No. 62/569,437, filed on Oct. 6, 2017, which is incorporated herein in its entirety for all purposes.

### STATEMENT OF GOVERNMENTAL SUPPORT

This invention was made with government support under Grant Nos. AG009909 and AG051729 awarded by the National Institutes of Health. The Government has certain rights in this invention.

### BACKGROUND

Apoptosis and senescence have been proposed to be two processes that prevent the emergence of malignantly transformed cells. Senescence is typically a permanent cell cycle arrest in which cells remain metabolically active and adopt characteristic phenotypic changes. Senescent cells often appear large and extended, and exhibit spindle and vacuolization features. The establishment of this phenotype is believed, inter alia, to be the result of telomere shortening after a number of cell divisions, which cells perceive as DNA damage, or a response to stressful stimuli such as non-telomeric DNA damage, mitochondrial dysfunction, hyperproliferation and metabolic imbalances. Expression of oncogenes or cell cycle stimulators, such as mutant Ras, cyclin E, E2F3 and mutant Raf can also trigger a senescence response, which supports the tumor suppressing properties of senescence.

The presence of senescent cells in an individual is thought to contribute to aging and aging-related dysfunction (see, e.g., Campisi (2005) Cell, 120: 513-522). Senescent cells accumulate in tissues and organs of individuals as they age and are found at sites of age-related pathologies. Moreover, at least in mouse models, it is clear that senescent cells drive a growing and diverse number of age-related diseases (see, e.g., Chinta et al. (2018) Cell Repts. 22: 930-940; Childs et al. (2016) Science, 354(6311): 472-477; Baker et al. (2016) Nature, 530(7589): 184-189; Chang et al. (2016) Nat. Med. 22(1): 78-83).

The presence of senescent cells in vivo is often observed in the pre-malignant stages of tumor formation and they frequently progressively disappear as tumors develop, suggesting that the senescent barrier needs to be overcome in order to progress into full malignancy. Moreover, cellular senescence has long been associated with age-dependent organismal changes since the accumulation of senescent cells has been shown to contribute to the functional impairment of different organs. This has led to the hypothesis that senescence is an antagonistically pleiotropic process, with beneficial effects in the early decades of life of the organism as a tumor suppressor but with detrimental effects on fitness and survival in later stages of life as a driver of aging.

Cellular senescence is also associated with the secretion of growth factors, chemokines and cytokines, (and now biologically active lipids such as eicosanoids as described herein) known as the senescence-associated secretary phenotype (SASP). SASPs have shown an effect on cell proliferation and angiogenesis, as well as in promoting ageing. Also, SASPs can induce the migration of leukocytes and tumor cells, which in turn may induce tumor metastasis. Increased expression of intracellular and/or secreted proteins has often been used as a surrogate marker of senescence, although it is none are an unequivocally specific measurement.

Given that senescent cells have been causally implicated in certain aspects of age-related decline in health and may contribute to certain diseases, and are also induced as a result of necessary life-preserving chemotherapeutic and radiation treatments, the presence of senescent cells may have deleterious effects on millions of patients worldwide.

Several labs are developing drugs that selectively target senescent cells and eliminate them (senolytic drugs). Transgenic mouse models already exist that allow selective elimination of senescent cells. In transgenic mice, the efficacy of elimination can be assessed owing to a reporter on the transgene. In non-transgenic mice—and humans, of course—there is a need to be able to assess whether and to what extent a senolytic drug is efficacious.

### SUMMARY

Various biological markers that function as indicators of the level of senescent cells in an organism are provided. In certain embodiments, the markers described herein (e.g., certain eicosanoids) can provide effective indicators of the presence and/or quantity of senescent cells in a subject (e.g., in a human or non-human mammal) and methods of identifying elevated levels of senescent cells in a mammal, are provided.

Various embodiments contemplated herein may include, but need not be limited to, one or more of the following:

### Embodiment 1

A method of identifying elevated levels of senescent cells in a mammal, said method comprising:
determining the levels of one or more indicators of senescent cells in a biological sample from said mammal, wherein said one or more indicators are selected from the group consisting of an eicosanoid, an eicosanoid precursor, leukotriene A4 (LTA4), leukotriene B4 (LTB4), PGD2, and 5-HETE; and
wherein an elevated level of said one or more indicators is an indicator of elevated levels of senescent cells in said mammal.

### Embodiment 2

The method of embodiment 1, wherein said indicators comprise one or more indicators selected from the group consisting of an eicosanoid, an eicosanoid precursor, leukotriene A4 (LTA4), and leukotriene B4 (LTB4).

### Embodiment 3

The method according to any one of embodiments 1-2, wherein said elevated level is as compared to a normal healthy mammal.

### Embodiment 4

The method according to any one of embodiments 1-3, wherein an elevated level is a statistically significant elevated level.

### Embodiment 5

The method according to any one of embodiments 1-4, wherein said indicators comprises an eicosanoid, or an eicosanoid precursor.

### Embodiment 6

The method of embodiment 5, wherein said indicator(s) comprises an eicosanoid.

### Embodiment 7

The method of embodiment 6, wherein said indicators comprise 1a,1b-dihomo-15-deoxy-delta12,14-prostaglandin J2 (dihomo-15d-PGJ2). It is noted that PGJ2 is intracellular and so is released when senescent cells are lysed as a result of senolysis.

### Embodiment 8

The method of embodiment 5, wherein said indicator(s) comprise an eicosanoid precursor.

### Embodiment 9

The method of embodiment 8, wherein said indicator(s) comprise an eicosanoid precursor selected from the group consisting of arachidonic acid (AA), eicosapentanoic acid (EPA), and dihomo-gamma-linoleic acid (DGLA).

### Embodiment 10

The method of embodiment 9 wherein said indicator(s) comprise arachidonic acid (AA).

### Embodiment 11

The method of embodiment 9 wherein said indicator(s) comprise eicosapentanoic acid (EPA).

### Embodiment 12

The method of embodiment 9 wherein said indicator(s) comprise dihomo-gamma-linoleic acid (DGLA).

### Embodiment 13

The method according to any one of embodiments 1-12, wherein said mammal is a human.

### Embodiment 14

The method according to any one of embodiments 1-12, wherein said mammal is a non-human mammal.

### Embodiment 15

The method according to any one of embodiments 1-12, wherein said biological sample comprises a sample selected from the group consisting of blood or a blood fraction, urine, cerebrospinal fluid, a tissue biopsy, an oral fluid, and a nasal, buccal swab, lavage fluids (e.g., brochoalveolar lavage), synovial fluid, lymph, pericardial fluid, and interstitial fluid.

### Embodiment 16

The method of embodiment 15, wherein said sample comprises blood or a blood fraction.

### Embodiment 17

The method of embodiment 15, wherein said sample comprises urine.

### Embodiment 18

The method according to any one of embodiments 1-17, wherein said indicator(s) are determined using one or more methods selected from the group consisting of an immunoassay, mass spectrometry, and HPLC.

### Embodiment 19

The method according to any one of embodiments 1-18, wherein said indicator(s) are determined using an ELISA.

### Embodiment 20

The method according to any one of embodiments 1-19, wherein said indicator(s) are determined as components of a differential diagnosis for a pathology characterized by elevated levels of senescent cells.

### Embodiment 21

The method of embodiment 20, wherein said pathology comprises a pathology selected from the group consisting of a cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke), a neurodegenerative disease (e.g., Alzheimer's disease, Parkinson's disease, Huntington's disease, dementia, mild cognitive impairment, and motor neuron dysfunction), a metabolic disease (e.g., diabetes, diabetic ulcer, metabolic syndrome, and obesity), and a senescence-associated disease.

### Embodiment 22

The method of embodiment 21, wherein said pathology comprises a senescence-associated disease that comprises a pulmonary disorder e.g., pulmonary fibrosis, chronic obstructive pulmonary disease, asthma, cystic fibrosis, emphysema, bronchiectasis, and age-related loss of pulmonary function.

### Embodiment 23

The method of embodiment 21, wherein said pathology comprises a senescence-associated disease that comprises an eye disease (e.g., macular degeneration, glaucoma, cataracts, presbyopia, and vision loss).

### Embodiment 24

The method of embodiment 21, wherein said pathology comprises a senescence-associated disease that comprises an age-related disorder selected from the group consisting of renal disease, renal failure, frailty, hearing loss, muscle fatigue, skin conditions, skin wound healing, liver fibrosis, pancreatic fibrosis, oral submucosa fibrosis, and sarcopenia.

### Embodiment 25

The method of embodiment 21, wherein said pathology comprises a senescence-associated disease that comprises a dermatological disease or disorder (e.g., eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, cutaneous lupus, etc.).

### Embodiment 26

The method of embodiment 21, wherein said pathology comprises a senescence-associated disease selected from the group consisting of atherosclerosis, osteoarthritis, pulmonary fibrosis, hypertension, and chronic obstructive pulmonary disease.

### Embodiment 27

A method of treating a pathology characterized by elevated levels of senescent cells in a mammal, said method comprising: administering an effective amount of one or more senolytic agents to a mammal determined to have elevated levels of one or more indicators of senescent cells wherein said one or more indicators are selected from the group consisting of an eicosanoid, an eicosanoid precursor, leukotriene A4 (LTA4), leukotriene B4 (LTB4), and 5-HETE.

### Embodiment 28

The method of embodiment 27, wherein said mammal is determined to have elevated levels of one or more indicators using the methods according to any one of embodiments 1-26.

### Embodiment 29

The method according to any one of embodiments 27-28, wherein administering comprises administering a therapeutically effective course of therapy of a small molecule senolytic agent wherein the senolytic agent selectively kills senescent cells in comparison with non-senescent cells.

### Embodiment 30

The method according to any one of embodiments 27-29, wherein the senolytic agent is a specific inhibitor of MDM2, Bcl-xL or Akt.

### Embodiment 31

The method according to any one of embodiments 27-29, wherein the senolytic agent comprises an inhibitor of Bcl-xL or Bcl-2.

### Embodiment 32

The method according to any one of embodiments 27-29, wherein the senolytic agent comprises an inhibitor of MDM2.

### Embodiment 33

The method of embodiment 32, wherein the MDM2 inhibitor comprises an imidazoline compound (e.g., a cis-imidazoline compound), a dihydroimidazothiazole compound, a spiro-oxindole compound, a benzodiazepine compound, or a piperidinone.

### Embodiment 34

The method of embodiment 32, wherein the MDM2 inhibitor is selected from the group consisting of Nutlin-1, Nutlin-2, RG-7112, RG7388, RO5503781, DS-3032b, MI-63, MI-126, MI-122, MI-142, MI-147, MI-18, MI-219, MI-220, MI-221, MI-773, 3-(4-chlorophenyl)-34(1-(hydroxymethyl)cyclopropyl)methoxy)-2-(4-nitrobenzyl)isoindolin-1-one, Serdemetan, AM-8553, CGM097, RO-2443, and RO-5963.

### Embodiment 35

The method of embodiment 33, wherein the senolytic agent comprises an imidazoline compound.

### Embodiment 36

The method of embodiment 35, wherein the imidazoline compound comprises a compound having the structure:

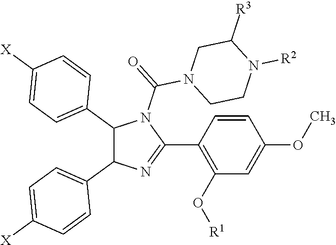

or a pharmaceutically acceptable salt thereof; wherein: X is halide; R¹ is alkyl, R² is —H or heteroalkyl, and R³ is —H or ═O.

### Embodiment 37

The method of embodiment 36, wherein the imidazoline compound is selected from the group consisting of nutlin-1, nutlin-2, and nutlin-3.

### Embodiment 38

The method of embodiment 36, wherein the imidazoline compound comprises a 4-[[(4S,5R)-4,5-bis(4-chlorophenyl)-4,5-dihydro-2-[4-methoxy-2-(1-methyle-thoxy)phenyl]-1H-imidazol-1-yl]carbonyl]-2-piperazinone or a pharmaceutically acceptable salt thereof.

### Embodiment 39

The method of embodiment 35, wherein the imidazoline compound comprises a compound having the structure:

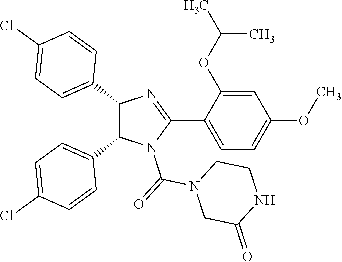 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

or a pharmaceutically acceptable salt thereof.

### Embodiment 40

The method according to any one of embodiments 27-39, wherein the senolytic agent is administered to the mammal as a monotherapy.

### Embodiment 41

The method according to any one of embodiments 27-40, wherein the administering comprises administering an amount of the senolytic agent and/or a frequency of dosage that is less than would be effective for treating cancer.

### Embodiment 42

The method according to any one of embodiments 27-41, wherein the administering comprises a period of treatment followed by a non-treatment interval of at least two weeks.

### Embodiment 43

The method according to any one of embodiments 27-41, wherein the administering comprises at least two treatment cycles of the senolytic agent, each treatment cycle independently including a treatment period of one day to three months followed by the non-treatment interval.

### Embodiment 44

The method according to any one of embodiments 27-41, wherein the administering comprises a single dose of the senolytic agent followed by the non-treatment interval.

### Embodiment 45

The method according to any one of embodiments 27-44, wherein said pathology comprises a pathology selected from the group consisting of a cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke), a neurodegenerative disease (e.g., Alzheimer's disease, Parkinson's disease, Huntington's disease, dementia, mild cognitive impairment, and motor neuron dysfunction), a metabolic disease (e.g., diabetes, diabetic ulcer, metabolic syndrome, and obesity), and a senescence-associated disease.

### Embodiment 46

The method of embodiment 45, wherein said pathology comprises a senescence-associated disease that comprises a pulmonary disorder (e.g., pulmonary fibrosis, chronic obstructive pulmonary disease, asthma, cystic fibrosis, emphysema, bronchiectasis, and age-related loss of pulmonary function.

### Embodiment 47

The method of embodiment 45, wherein said pathology comprises a senescence-associated disease that comprises an eye disease (e.g., macular degeneration, glaucoma, cataracts, presbyopia, and vision loss).

### Embodiment 48

The method of embodiment 45, wherein said pathology comprises a senescence-associated disease that comprises an age-related disorder selected from the group consisting of renal disease, renal failure, frailty, hearing loss, muscle fatigue, skin conditions, skin wound healing, liver fibrosis, pancreatic fibrosis, oral submucosa fibrosis, and sarcopenia.

### Embodiment 49

The method of embodiment 45, wherein said pathology comprises a senescence-associated disease that comprises a dermatological disease or disorder (e.g., eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, cutaneous lupus, etc.).

### Embodiment 50

The method of embodiment 45, wherein said pathology comprises a senescence-associated disease selected from the group consisting of atherosclerosis, osteoarthritis, pulmonary fibrosis, hypertension, and chronic obstructive pulmonary disease.

### Embodiment 51

The method according to any one of embodiments 27-50, wherein the senolytic agent is administered locally at or near the site of the disease or disorder.

### Embodiment 52

The method of embodiment 51, wherein the senolytic agent is administered to an osteoarthritic joint.

### Embodiment 53

The method according to any one of embodiments 27-52, wherein said mammal is a human.

### Embodiment 54

The method according to any one of embodiments 27-52, wherein said mammal is a non-human mammal.

### Embodiment 55

The method according to any one of embodiments 27-54, wherein said method (preferentially) reduces the levels of senescent cells in said mammal.

### Embodiment 56

A method of evaluating the efficacy of a treatment of a pathology characterized by elevated senescent cells, said method comprising:
determining a first level of one or more indicators of senescent cells in said mammal using a method according to any one of embodiments 1-26;
treating said mammal using a method according to any one of embodiments 27-55; and
determining a second level of one or more indicators of senescent cells in said mammal after or during said treating, using a method according to any one of embodiments 1-26 wherein a decrease in the second level of said indicator(s) as compared to the first level of said indicators indicates that said treatment is effective and the absence of change in level or an increase in the second level of said indicator(s) as compared to the first level of said indicators indicates that said treating is not effective.

### Embodiment 57

The method of embodiment 56, wherein said method comprises detecting active senolysis.

### Embodiment 58

The method according to any one of embodiments 56-57, wherein said method comprises detecting Dihomo-15d-PGJ2, and/or 15d-PGJ2 in urine as a marker of active senolysis in said mammal.

With respect to the embodiments described herein, it is noted that the use of dihomo-15d-PGJ2, and/or 15d-PGJ2) as biomarkers of senolysis (see, e.g., FIG. 14) during senolytic treatment is a major finding of the work described herein. It is believed these are the first biomarkers of its their kind (e.g., as seen in the urine data). Accordingly, one focus of the work described herein is the detection of active senolysis in fluids during the process, showing that senolytic drugs are actually working.

### Definitions

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that is characterized by elevated levels of senescent cells and/or a pathology characterized by elevated levels of senescent cells.

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term treat can refer to prophylactic treatment, which includes a delay in the onset or the prevention of the onset of a pathology or disease.

A “senescent cell” may exhibit any one or more of the following seven characteristics. (1) Senescence growth arrest is essentially permanent and cannot be reversed by known physiological stimuli. (2) Senescent cells increase in size, sometimes enlarging more than twofold relative to the size of non-senescent counterparts. (3) Senescent cells express a senescence-associated β-galactosidase (SA-β-gal), which partly reflects the increase in lysosomal mass. (4) Most senescent cells express p16INK4a, which is not commonly expressed by quiescent or terminally differentiated cells. (5) Cells that senesce with persistent DNA damage response (DDR) signaling harbor persistent nuclear foci, termed DNA segments with chromatin alterations reinforcing senescence (DNA-SCARS). These foci contain activated DDR proteins and are distinguishable from transient damage foci. DNA-SCARS include dysfunctional telomeres or telomere dysfunction-induced foci (TIF). (6) Senescent cells express and may secrete molecules associated with senescence, which in certain instances may be observed in the presence of persistent DDR signaling, which in certain instances may be dependent on persistent DDR signaling for their expression. (7) The nuclei of senescent cells lose structural proteins such as Lamin B1 or chromatin-associated proteins such as histones and HMGB1 (see, e.g., Freund et al. (2012) Mol. Biol. Cell, 23: 2066-2075; Davalos et al. (2013) J. Cell Biol. 201: 613-629; Ivanov et al. (2013) J. Cell Biol. 202(1):129-43). (8). Senescent cells are also characterized by senescent cell-associated secreted molecules, which include growth factors, proteases, cytokines (e.g., inflammatory cytokines), chemokines, cell-related metabolites, reactive oxygen species (e.g., H₂O₂), and other molecules that stimulate inflammation and/or other biological effects or reactions that may promote or exacerbate the underlying disease of the subject. Senescent cell-associated molecules include those that are described in the art as comprising the senescence-associated secretory phenotype (SASP, e.g., which includes secreted factors that may make up the pro-inflammatory phenotype of a senescent cell), senescent-messaging secretome, and DNA damage secretory program (DDSP). These groupings of senescent cell associated molecules, as described in the art, contain molecules in common and are not intended to describe three separate distinct groupings of molecules. Senescent cell-associated molecules include certain expressed and secreted growth factors, proteases, cytokines, and other factors that may have potent autocrine and paracrine activities (see, e.g., Coppe et al. (2006) J. Biol. Chem. 281: 29568-29574; Coppe et al. (2010) PLoS One, 5: 39188; Krtolica et al. (2001) Proc. Natl. Acad. Sci. USA, 98: 12072-12077; Parrinello et al. (2005) J. Cell Sci. 118: 485-496). Extracellular matrix (ECM) associated factors include inflammatory proteins and mediators of ECM remodeling and which are strongly induced in senescent cells (see, e.g., Kuilman et al. (2009) Nat. Rev., 9: 81-94). Other senescent cell-associated molecules include extracellular polypeptides (proteins) described collectively as the DNA damage secretory program (DDSP) (see, e.g., Sun et al. (2012) Nat. Med., 18: 1359-1368). Senescent cell-associated proteins also include cell surface proteins (or receptors) that are expressed on senescent cells, which include proteins that are present at a detectably lower amount or are not present on the cell surface of a non-senescent cell. Senescence cell-associated molecules include secreted factors that may make up the pro-inflammatory phenotype of a senescent cell (e.g., SASP). These factors include, without limitation, GM-CSF, GROα, GROα, β, γ, IGFBP-7, IL-1α, IL-6, IL-7, IL-8, MCP-1, MCP-2, MIP-1α, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-13, MCP-4, MIF, MIP-3α, MMP-12, MMP-13, MMP-14, NAP2, Oncostatin M, osteoprotegerin, PIGF, RANTES, sgp130, TIMP-2, TRAIL-R3, Acrp30, angiogenin, Axl, bFGF, BLC, BTC, CTACK, EGF-R, Fas, FGF-7, G-CSF, GDNF, HCC-4, 1-309, IFN-γ, IGFBP-1, IGFBP-3, IL-1 R1, IL-11, IL-15, IL-2R-α, IL-6 R, I-TAC, Leptin, LIF, MMP-2, MSP-a, PAI-1, PAI-2, PDGF-BB, SCF, SDF-1, sTNF RI, sTNF MI, Thrombopoietin, TIMP-1, tPA, uPA, uPAR, VEGF, MCP-3, IGF-1, TGF-β3, MIP-1-delta, IL-4, FGF-7, PDGF-BB, IL-16, BMP-4, MDC, MCP-4, IL-10, TIMP-1, Fit-3 Ligand, ICAM-1, Axl, CNTF, INF-γ, EGF, BMP-6. Additional identified factors, which include those sometimes referred to in the art as senescence messaging secretome (SMS) factors, some of which are included in the listing of SASP polypeptides, include without limitation, IGF1, IGF2, and IGF2R, IGFBP3, IDFBP5, IGFBP7, PA11, TGF-β, WNT2, IL-1α, IL-6, IL-8, and CXCR2-binding chemokines. Cell-associated molecules also include without limitation the factors described in Sun et al., Nat. Med, supra, and include, including, for example, products of the genes, MMP1, WNT16B, SFRP2, MMP12, SPINK1, MMP10, ENPP5, EREG, BMP6, ANGPTL4, CSGALNACT, CCL26, AREG, ANGPT1, CCK, THBD, CXCL14, NOV, GAL, NPPC, FAMI50B, CST1, GDNF, MUCLJ, NPTX2, TMEM155, EDNJ, PSG9, ADAMTS3, CD24, PPBP, CXCL3, MMP3, CST2, PSG8, PCOLCE2, PSG7, TNFSF15, C17orf67, CALCA, FGF18, IL8, BMP2, MATN3, TFP1, SERPINI 1, TNFRSF25, and IL23A. Senescent cell-associated proteins also include cell surface proteins (or receptors) that are expressed on senescent cells, which include proteins that are present at a detectably lower amount or are not present on the cell surface of a non-senescent cell.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110: 4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments. In addition, it is possible that nucleic acids of the present invention can alternatively be triple-stranded.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer, which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331). In certain embodiments antibodies also include peptibodies. Peptibodies consist of biologically active peptides grafted onto an Fc domain. This approach retains certain desirable features of antibodies, notably an increased apparent affinity through the avidity conferred by the dimerization of two Fcs and a long plasma residency time (see, e.g., Shimamoto et al. (2012) Mabs, 4(5): 586-591).

The term “biological sample” refers to sample that is a sample of biological tissue, cells, or fluid that, in a healthy and/or pathological state, contains one or more of the indicators of senescent cells described herein. Such samples include, but are not limited to, cultured cells, acute cell preparations, sputum, amniotic fluid, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Although the sample is typically taken from a human patient, the assays can be used on samples from any mammal, such as dogs, cats, sheep, cattle, and pigs, etc. The sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

The term “small organic molecule” refers to a molecule of a size comparable to those organic molecules generally used in pharmaceuticals. The term excludes biological macromolecules (e.g., proteins, nucleic acids, etc.). In certain embodiments preferred small organic molecules range in size up to about 5000 Da, or up to about 4000 kDa, or up to about 3,000 kDa, or up to about 2000 Da, or up to about 1000 Da.

### BRIEF DESCRIPTION OF THE DRAWINGS

FIG. 1, panels A-I, shows that senescent cells synthesize eicosanoids. Panels A-B: Lipids were extracted from proliferating (PRO—10%), quiescent (QUI—0.2%), and IR-induced senescent (SEN(IR)—10% or 0.2%) IMR-90 fibroblasts and were analyzed by liquid chromatography combined with mass spectrometry (LC-MS). Eicosanoids (panel A) and lipid precursors (panel B) were detected in control and senescent cells. Panel C: Protein was extracted from QUI or SEN(IR) cells and analyzed by immunoblot for cPLA2 (phosphorylated on serine 505 or total cPLA2), p38MAPK (phosphorylated or total), and tubulin. Panel D: RNA was isolated from QUI and SEN(IR) cells, reverse transcribed, and eicosanoid synthesis gene expression was measured by quantitative PCR. Panels E-F: RNA was isolated from cells at various time points after IR (10 Gy) and analyzed by quantitative PCR for PTGS2 (panel E) or ALOX5 (panel F). Panel G: Fragmentation pattern of a 15d-PGJ2 standard. Panels H-I: Mass spectra of 15d-PGJ2 (panel H) and dihomo-15d-PGJ2 (panel I) measured in senescent and proliferating cells.

FIG. 2, panels A-F, shows that prostaglandins promote the SASP. Panel A: IMR-90 fibroblasts were induced to senesce by ionizing radiation (IR) and treated with DMSO, COX-2 inhibitors—CAY-10404 (CAY) or NS-398 (NS), ALOX5 inhibitors—zileuton (Zil) or BW-B70C (BW), or combinations of each type of inhibitor for 10 days. Conditioned media were harvested and secreted IL-6 was measured by ELISA. Panel B: Cells were treated as in A, and SASP mRNA levels were measured by quantitative PCR. Panel C: NF-κB luciferase reporter activity was measured in SEN(IR) cells treated with either DMSO or CAY-10404 and BW-B70C. Panel D: Proliferating cells were cultured for 10 days in the presence of 10 μM of prostaglandins PGA2, PGD2, PGE2, PGF2α, or PGJ2. Conditioned media were harvested and secreted IL-6 was measured by ELISA. Panels E-F: Cells were treated as in panel D, RNA was extracted, and SASP and PTGS levels were measured by quantitative PCR (panel E), PGJ2 treatment is shown independently (panel F) due to an exponentially increased level of induction.

FIG. 3, panels A-L, shows that prostaglandins reinforce cell cycle arrest during senescence. Panels A-C: IMR-90 fibroblasts were irradiated with 5 Gy IR and cultured for ten days in zileuton, CAY-10404, NS-398, CAY-10404 and zileuton (CAY+Zil), NS-398 and zileution (NS+Zil). Cells were then assayed for (panel A) cell number, (panel B) senescence-associated beta-galactosidase (SA-B-gal), and (panel C) p21 mRNA levels normalized to tubulin. Panels D-F: Proliferating cells were cultured for 10 days in the presence of 10 μM of prostaglandins PGA2, PGD2, PGE2, PGF2α, PGJ2, or 15d-PGJ2 and assayed for (panel D) EdU labeling (24 h), (panel E) SA-B-gal, or (panel F) p21 mRNA levels normalized to tubulin. Panel G: Cells were treated as in panel D and protein lysates were analyzed by immunoblot for LMNB1, p53, HMGB1, p21, and actin. H-I. IMR-90 fibroblasts were transduced with viruses expressing shRNAs to either GFP (shGFP) or p53 (shp53) and treated with either PGD2 or PGJ2. After 3 days, cells were fixed and stained for either (H) KI-67, or (I) cleaved caspase 3 and scored according to positivity. Panels J-L: Cells were treated with 1.4 μM 15d-PGJ2 and irradiated with different doses of IR. After 10 days, cells were scored for (panel J) SA-B-gal, (panel K) EdU labeling (24 h), or (panel L) cell number.

FIG. 4, panels A-J, shows that senescence-associated leukotriene synthesis promotes pulmonary fibrosis. C57BL/6 and p16-3MR mice were administered either PBS or bleomycin (Bleo) intratracheally. From day 7 after bleomycin until analysis, mice received by gavage 50 mg/kg/day ABT-263 or vehicle (Veh) or by I.P. 25 mg/kg/day ganciclovir (GCV). Panel A: RNA expression of p16^{INNK4a} in lungs from mice at day 14. Panel B: p21^{WAF1} (CDKN1A) expression in lungs from treated mice at day 14. Panel C: Hydroxyproline levels in lungs from mice 21 days after bleomycin instillation. Panels D-E: RNA expression of (panel D) COL3A1 or (panel E) COL4A1 in lungs from treated mice on day 14. Panel F: Picrosirius red staining of lungs treated as in panel C. Panel G: Heat map indicating expression of ALOX5, LTC4S, PTGDS, PTGS2, and PTGES 14 days after bleomycin injury in lungs from treated mice. Panel H: Ratio of phosphorylated cPLA2 to total cPLA2 in western lysates from treated mice on day 14. Panel I: Lipids were extracted from day 14 BALF of treated mice and analyzed for cysteinyl leukotrienes by ELISA. Panel J: Lipids from I were analyzed for PGE2 by ELISA.

FIG. 5, panels A-E, shows that temporal changes in eicosanoid biosynthesis reveal pro- and anti-fibrotic effects of senescent cells. Panels A-B: C57BL/6 and p16-3MR mice were administered either PBS (Day 0) or bleomycin (Bleo) intratracheally, and RNA was extracted from tissue at the indicated time points. Panel A: RNA levels of p16^{INK4a} (p16, blue line) and collagen (Col1a2, red line). Panel B: RNA levels of Alox5 (blue line) and Ptgds (red line). Panels C-D: Conditioned media (CM) from senescent (2 or 20 days after IR) or non-senescent (0 days) cells treated with either DMSO, NS-398, or zileuton for 24 h prior to generation of conditioned media. Media were the applied to naïve nonsenescent IMR-90 fibroblasts in the presence of TGF-beta or carrier (BSA). After 24 h treatment, RNA was extracted from treated cells and analyzed by qPCR for (panel C) Col1a2 or, (panel D) Acta2. Panel E: IMR-90 or LL-29 fibroblasts were induced to senescence by 10 Gy IR, and analyzed at day 10 for expression of ALOX5, PTGS2, PTGDS, and PTGES by qPCR.

FIG. 6, panels A-C, shows eicosanoid biosynthesis enzyme levels change over time. IMR-90 fibroblasts were induced to senesce by 10 Gy ionizing radiation (IR) and mRNA was harvested at the indicated number of days after irradiation. Gene expression of PTGES (panel A), LTA4H (panel B), and LTC4S (panel C) was measured by quantitative PCR for each time point.

FIG. 7 illustrates biosynthetic pathways that promote synthesis of dihomo-15d-PGJ2 and 15dPGJ2 in senescent cells. Arrows indicate steps in the synthesis of 15d-PGJ2. Bold letters indicate biosynthetic intermediates. Green letters indicate fold changes in biosynthetic enzymes elevated at the mRNA level during senescence. Bar graphs demonstrate relative levels of the intermediates along the 15d-PGJ2 biosynthetic pathways. Purple lettering indicates non-enzymatic dehydration reactions.

FIG. 8, panels A-D, shows that eicosanoids control the SASP. Panel A: IMR-90 fibroblasts were transfected with a PPARgamma luciferase reporter. After selection, cells were irradiated (IR) and treated with either a vehicle (DMSO) or NS-398 for 10 days and extracts were analyzed by luminometry. Panel B: Mock-irradiated and senescent SEN(IR) cells were analyzed by qPCR for CYSLTR2 and LTB4R2. Panel C: IMR-90 fibroblasts were induced to senesce by 10 Gy ionizing radiation (IR) and treated with zileuton (Zil), NS-398 (NS), CAY-10404 (CAY), CAY+Zil, NS+Zil, or vehicle (DMSO) for 10 days. RNA was extracted and analyzed by quantitative PCR (qPCR). Panel D: Cells were irradiated (IR) and treated with either a vehicle (DMSO) or CAY-10404 for 10 days and RNA was analyzed by qPCR for PTGS2. Panel D: Proliferating cells were treated with 10 μM of 5-HETE, LTB4, LTC4, LTD4, LTE4 or vehicle (EtOH) and analyzed by qPCR.

FIG. 9, panels A-D, shows that eicosanoids promote oncogene-induced senescence. Proliferating IMR-90 fibroblasts were transduced with a RasV12-overexpressing lentivirus and treated for 7 days with zileuton, NS-398, CAY-10404, NS-398+Zileuton, or vehicle (DMSO). Panel A: IL-6 ELISA on conditioned media from treated cells. Panel B: 24 hour EdU incorporation indices for treated cells. Panels C-D: 1000 cells from A were re-plated and cultured for an additional 7 days in identical treatments, and wells were stained with crystal violet and analyzed for (panel C) percentages of well areas covered by colonies and (panel D) representative images of wells from each treatment group.

FIG. 10, panels A-F, shows the reversibility of prostaglandin-induced senescent phenotypes. Panels A-B: Proliferating IMR-90 fibroblasts were cultured in the presence of 10 μM PGD2, PGE2, or PGJ2 for 7 days, and then subcultured for an additional 10 days in the presence (Hold) or absence (Release) of the same prostaglandin and analyzed for SA-B-gal labeling (panel A) or EdU 24 hour incorporation (panel B). Panels C-E: Cells were treated with DMSO, 10 μM PGD2, or 10 μM PGJ2, as in A, RNA was then extracted and analyzed by qPCR for (panel C) p21^{WAF1} (CDKN1A), (panel D) LMNB1, or (panel E) MMP3. Panel F: IL-6 ELISA on conditioned media from cells treated as in panels C-E.

FIG. 11 shows that p53 is stabilized following PGD2 or PGJ2 treatment without detectable posttranslational modification. Proliferating IMR-90 fibroblasts were treated with 10 μM PGA2, PGD2, PGE2, PGF2α, PGJ2, or vehicle (DMSO) for 10 days, follow by analysis by western blot for total p53, p53 phospho-S15, p53 phospho-S33, p53 phospho-S37, p53 phospho-S15, p53 phospho-S392, p53 acetyl-K320, p53 acetyl-K382, or beta-actin.

FIG. 12, panels A-B, shows that 15d-PGJ2 induces apoptosis in breast cancer cells with mutant p53. MCF7 or MDA-MB-231 breast cancer cells were treated with 10 μM 15d-PGJ2 for 3 days. Cells were then fixed and stained for cleaved caspase-3 by immunofluorescence. Panel A: Representative images of cleaves caspase-3 staining. Panel B: Quantitation of caspase positivity.

FIG. 13, panels A-F, illustrates elevated markers of eicosanoid biosynthesis in mice exposed to senescence inducers. Panel A: p16-3MR mice were treated with a single bolus of doxorubicin (DOXO) or phosphate-buffered saline (PBS) by i.p. injection. After 5 days, mice were treated with either GCV or vehicle for 5 days. Livers were harvested on day 10 and analyzed for eicosanoid synthase gene expression by qPCR. Panel B: p16-3MR mice were aged for 21 mo receiving GCV or PBS for 5 days each month by i.p. injection. Livers were harvested from these mice and 1 mo old controls and analyzed for eicosanoid synthase gene expression by qPCR. Panels C-F: Mice were treated with PBS or bleomycin by intratracheal administration and treated with either vehicle or ABT-263 from day 7 to day 13. Lungs were harvested for analysis on Day 14. Panel C: qPCR analysis of leukotriene synthases ALOX5 and LTC4S normalized to actin. Panel D: qPCR analysis of prostaglandin synthases PTGDS, PTGS2, and PTGES normalized to actin. Panel E: Western blots on lung extracts probed with antibodies to cPLA2 phospho-550. Panel F: Ratio of cPLA2-S505P/cPLA2.

FIG. 14, panels A-E, show that 15d-PGJ2 is a biomarker of senolysis. Panel A: IMR-90 fibroblasts were induced to senesce by mitochondrial dysfunction (MiDAS) or ionizing radiation [SEN(IR)]. 10 days after induction, cells were treated with DMSO 10 uM ABT-263, and 15d-PGJ2 was measured in conditioned media by ELISA. Panels B-C: HEPG2 (B) or HUVEC(C) cells were induced to senesce by IR, and conditioned media from DMSO or ABT-263 treatments were generated as in panel A. 15d-PGJ2 was measure by ELISA. Panels D-E: C57BL/6 mice were injected intraperitoneally with doxorubin (DOXO; 10 mg/kg) or PBS. 6 weeks later, ABT-263 (50 mg/kg) or vehicle (Veh) were administered by gavage. 3 hours after gavage, blood was collected by cardiac puncture (panel D). 12 hours after gavage urine was collected (panel E). Lipids were extracted from either fluid, and 15d-PGJ2 was measured by ELISA as in panel A.

### DETAILED DESCRIPTION

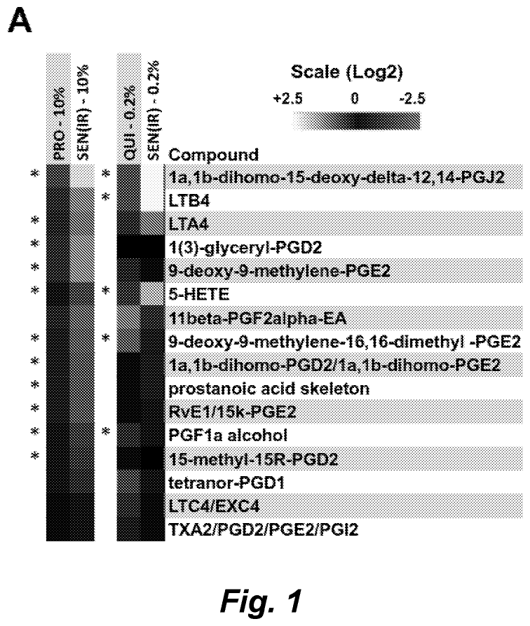

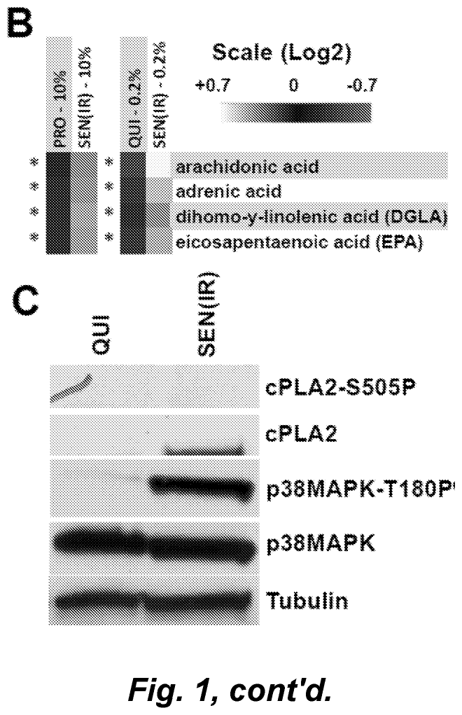

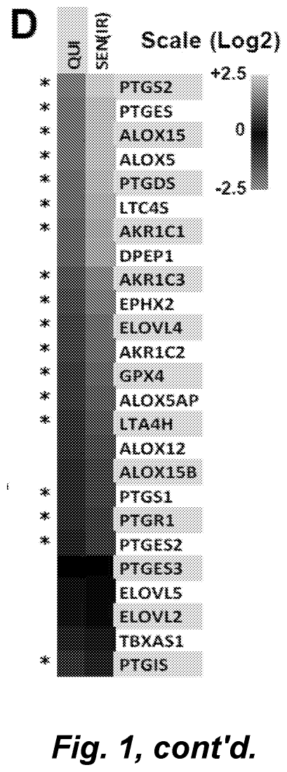

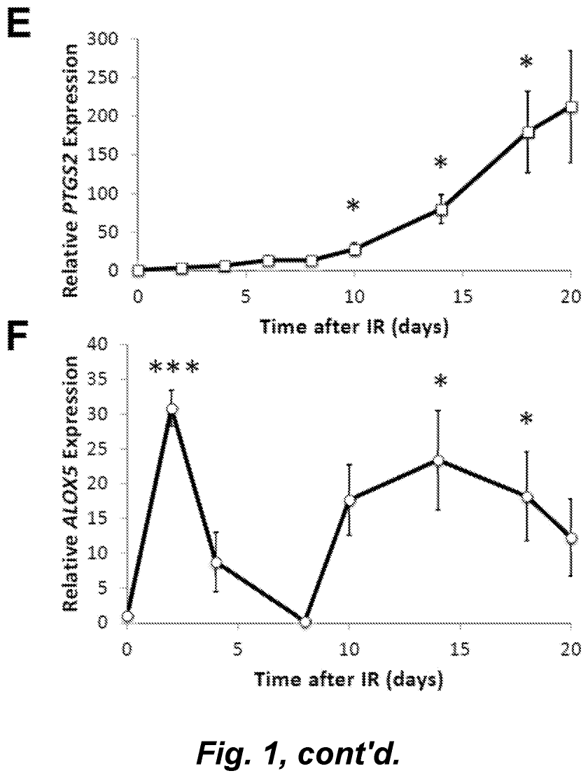

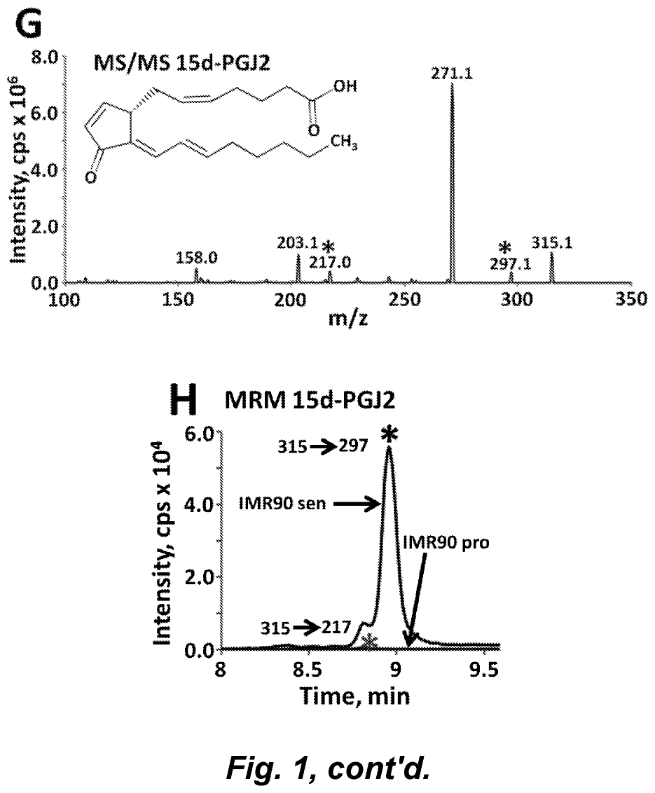

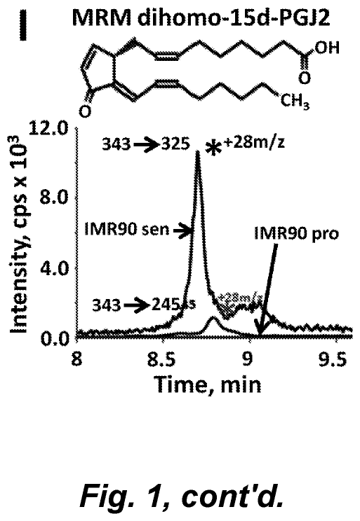

In order to better understand the metabolic changes that occur during cellular senescence, we extracted intracellular lipids and aqueous metabolites from proliferating, quiescent, and ionizing radiation (IR)-induced senescent fibroblasts and measured their relative abundances by mass spectrometry. It was discovered that that certain subsets of lipids showed strong elevation or decline with senescence. These included ceramides, saturated fatty acids, and retinoic acid. Most notable, however, were striking elevations in relative abundances of eicosanoids, a class of potent signaling lipids derived from 20-carbon fatty acids, most notably arachidonic acid. The most abundant of these eicosanoids was 1a,1b-dihomo-15-deoxy-delta12,14-prostaglandin J2 (dihomo-15d-PGJ2), but dihomo versions of prostaglandin D2 (PGD2) and prostaglandin E2 (PGE2) were also detected. Additionally, we observed increases in specific leukotrienes, notably leukotrienes A4 (LTA4) and B4 (LTB4), as well as the related lipoxygenase product, 5-HETE (FIG. 1, panel A). Additionally, the eicosanoid precursors arachidonic acid (AA), eicosapentanoic acid (EPA), and dihomo-gamma-linoleic acid (DGLA) were elevated in senescent cells, as was adrenic acid, a product of the elongation of AA and precursor of the dihomo prostaglandins. Thus, both eicosanoids and their precursors are elevated with senescence (the formation of senescent cells, e.g., cells characterized by the SASP phenotype).

In view of these discoveries the various markers described above can provide effective indicators of the presence and/or quantity of senescent cells in a subject (e.g., in a human or non-human mammal). Accordingly, in certain embodiments methods of identifying elevated levels of senescent cells in a mammal are provided. In certain embodiments the methods involve determining the levels of one or more indicators of senescent cells in a biological sample from the mammal (e.g., a blood or a blood fraction, urine, cerebrospinal fluid, a tissue biopsy, an oral fluid, a nasal or buccal swab, etc.), where the one or more indicators are selected from the group consisting of an eicosanoid, an eicosanoid precursor, leukotriene A4 (LTA4), leukotriene B4 (LTB4), PGD2, and 5-HETE. Elevated level(s) of the indicator(s) (e.g., as compared to the level(s) in a normal healthy mammal) is an indicator of elevated levels of senescent cells in the mammal. In certain embodiments the indicators comprise one or more indicators selected from the group consisting of an eicosanoid, an eicosanoid precursor, leukotriene A4 (LTA4), and leukotriene B4 (LTB4). In certain embodiments the indicator(s) comprise an eicosanoid, or an eicosanoid precursor. In certain embodiments the indicator(s) comprises an eicosanoid (e.g., 1a,1b-dihomo-15-deoxy-delta12,14-prostaglandin J2 (dihomo-15d-PGJ2)). In certain embodiments the indicator(s) comprise an eicosanoid precursor (e.g., arachidonic acid (AA), eicosapentanoic acid (EPA), and/or dihomo-gamma-linoleic acid (DGLA).

An elevated level of these one or more indicators alone, or in the context of a differential diagnosis, can be an indicator of a pathology characterized by elevated levels/numbers of senescent cells. Accordingly, in certain embodiments an elevated level of these one or more indicators can be an indication that the mammal has been successfully treated with one or more a senolytic agent(s). In various embodiments, such senolytic agents, as described herein, act to selectively and/or preferentially kill senescent cells and thus can be used as a prophylactic and/or therapeutic modality in subjects having an elevated level of senescent cells.

In certain embodiments the pathology characterized by elevated levels/numbers of senescent cells can include, but need not be limited to a pathology such as a cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke), a neurodegenerative disease (e.g., Alzheimer's disease, Parkinson's disease, Huntington's disease, dementia, mild cognitive impairment, and motor neuron dysfunction), a metabolic disease (e.g., diabetes, diabetic ulcer, metabolic syndrome, and obesity), or a senescence-associated disease.

The indicator(s) can routinely be determined in a biological sample using methods well known to those of skill in the art. Such methods include, but are not limited to, mass spectrometry (e.g., LC-MSI), chromatograph (e.g., HPLC), and the like (see, e.g., Example 1 herein).

### Senolytic Agents.

As noted above, in certain embodiments, the indicators described herein are surrogate markers for elevated levels of senescent cells. Subject having such elevated levels are candidates for intervention by administration of one or more senolytic agents that selectively/preferentially kill and/or inhibit senescent cells (e.g., cells characterized by a SASP phenotype).

Accordingly in creatine embodiments, the diagnostic methods described herein can further comprise administering an effective amount of one or more senolytic agents to a subject (e.g., to a mammal) determined to have elevated levels of one or more indicators of senescent cells as described herein. Similarly, in certain embodiments methods of treatment are contemplated where the methods comprise administering an effective amount of one or more senolytic agents to a subject (e.g., to a mammal) determined to have elevated levels of one or more indicators of senescent cells described herein (e.g., an eicosanoid, an eicosanoid precursor, leukotriene A4 (LTA4), leukotriene B4 (LTB4), PGD2, and/or 5-HETE).

In certain embodiments the indicator(s) described herein also facilitate evaluation of a treatment regimen involving the use of one or more senolytic agents. Accordingly, in certain embodiments such methods can involve determining a first level of one or more indicators of senescent cells (e.g., an eicosanoid, an eicosanoid precursor, leukotriene A4 (LTA4), leukotriene B4 (LTB4), PGD2, and/or 5-HETE) in a subject; treating the subject mammal using a method described herein (e.g., a method comprising the administration of one or more senolytic agents to the subject); and determining a second level of one the indicator(s) of senescent cells in the subject after or during the treatment where a decrease (e.g., a statistically significant decrease) in the second level of the indicator(s) as compared to the first level of the indicators indicates that the treatment is effective and the absence of change in level or an increase (e.g., a statistically significant increase) in the second level of the indicator(s) as compared to the first level of the indicators indicates that the treatment is not effective. In certain embodiments, where the treatment is deemed not effective the treatment regimen is altered. Such alteration can comprise a change in one or more senolytic agents administered, and/or a change in the dosage regimen.

Accordingly in various embodiments, the methods contemplated herein involve the administration of one or more senolytic agents to the subject at hand.

A senolytic agent as used herein is an agent that “selectively” (preferentially or to a greater degree) destroys, kills, removes, or facilitates selective destruction of senescent cells. In other words, in certain embodiments, the senolytic agent destroys or kills a senescent cell in a biologically, clinically, and/or statistically significant manner compared with its capability to destroy or kill a non-senescent cell. In typical embodiments, a senolytic agent is used in an amount and for a time sufficient to selectively kill established senescent cells but while substantially avoiding the killing or destruction of non-senescent cell(s) in a clinically significant or biologically significant manner. In certain embodiments, the senolytic agents alter at least one signaling pathway in a manner that induces (initiates, stimulates, triggers, activates, and/or promotes) and results in death of the senescent cell(s). The senolytic agent may alter, for example, either or both of a cell survival signaling pathway (e.g., an Akt pathway) or an inflammatory pathway, for example, by antagonizing a protein within the cell survival and/or inflammatory pathway in a senescent cell.

Without wishing to be bound by a particular theory, one mechanism by which the inhibitors and antagonists described herein selectively kill senescent cells is by inducing (e.g., activating, stimulating, removing inhibition of) one or more components of an apoptotic pathway that leads to cell death. In certain embodiments non-senescent cells may be proliferating cells or may be quiescent cells. In certain instances, exposure of non-senescent cells to the senolytic agent as used in the methods described herein may temporarily reduce the capability of non-senescent cell to proliferate, however, an apoptotic pathway is typically not induced and the non-senescent cell is typically not destroyed.

Certain senolytic agents that may be used in the methods described herein have been described as useful for treating a cancer. However, in the methods described herein, the senolytic agents are typically administered in a manner that would be considered different and likely ineffective for treating a cancer. The administration of one or more senolytic agents in the methods described herein may comprise one or more of a daily dose, cumulative dose over a single treatment cycle, or cumulative dose of the agent from multiple treatment cycles that is less than the dose of an agent required for cancer therapy. Therefore, the likelihood is decreased that one or more adverse effects (e.g., side effects) will occur that are associated with treating a subject according to a regimen optimized for treating a cancer. In various embodiments the senolytic agents administered in the methods described herein may be administered at a lower dose than presently described in the art for cancer treatment, and/or in a manner that selectively kill senescent cells (e.g., intermittent dosing). In certain embodiments, the senolytic agents described herein may be administered at a lower cumulative dose per treatment course or treatment cycle and would likely be insufficiently cytotoxic to cancer cells to effectively treat the cancer. In other words, according to the methods described herein, the senolytic agent is not used in a manner that would be understood by a person skilled in the art as a primary therapy for treating a cancer, whether the agent is administered alone or together with one or more additional chemotherapeutic agents or radiotherapy to treat the cancer.

However, in certain embodiments, even though an agent as used in the methods described herein is not used in a manner that is sufficient to be considered as a primary cancer therapy, the methods and senolytic combinations described herein may be used in a manner (e.g., a short term course of therapy) that is useful for inhibiting metastases. A “primary therapy for cancer” as used herein means that when an agent, which may be used alone or together with one or more agents, is intended to be or is known to be an efficacious treatment for the cancer as determined by a person skilled in the medical and oncology arts, administration protocols for treatment of the cancer using the agent have been designed to achieve the relevant cancer-related endpoints. In certain embodiments, to further reduce toxicity, a senolytic agent may be administered at a site proximal to or in contact with senescent cells (not tumor cells).

In certain embodiments the senolytic agents described herein alter (e.g., interfere with/inhibit) one or more cellular pathways that are activated during the senescence process of a cell. In certain embodiments senolytic agents may alter either cell survival signaling pathway (e.g., Akt pathway) and/or an inflammatory pathway in a senescent cell. Activation of certain cellular pathways during senescence decreases or inhibits the cell's capability to induce, and ultimately undergo apoptosis. Without wishing to be bound by theory, the mechanism by which a senolytic agent selectively kills senescent cells is by inducing (e.g., activating, stimulating, removing inhibition of) an apoptotic pathway that leads to cell death. In various embodiments a senolytic agent may alter one or more signaling pathways in a senescent cell by interacting with one, two, or more target proteins in the one or more pathways, which results in removing or reducing suppression of a cell death pathway, such as an apoptotic pathway. Contacting or exposing a senescent cell to a senolytic agent to alter one, two, or more cellular pathways in the senescent cell, may restore the cell's mechanisms and pathways for initiating apoptosis. In certain embodiments, a senolytic agent is an agent that alters a signaling pathway in a senescent cell, which in turn inhibits secretion and/or expression of one or more gene products important for survival of a senescent cell. The senolytic agent may inhibit a biological activity of the gene product(s) important for survival of the senescent cell. Alternatively, the decrease or reduction of the level of the gene product(s) in the senescent cell may alter the biological activity of another cellular component, which triggers, initiates, activates, or stimulates an apoptotic pathway or removes or reduces suppression of the apoptotic pathway. In certain embodiments senolytic agents contemplated herein comprise biologically active agents that are capable of selectively killing senescent cells in the absence of linkage or conjugation to a cytotoxic moiety (e.g., a toxin or cytotoxic peptide or cytotoxic nucleic acid). In certain embodiments the senolytic agents are also active in selectively killing senescent cells in the absence of linkage or conjugation to a targeting moiety (e.g., an antibody or antigen-binding fragment thereof; cell binding peptide) that selectively binds senescent cells.

In certain embodiments, a senolytic agent used in the methods described herein is a small organic molecule. In certain embodiments, the senolytic agents include those that are activated or that are pro-drugs that are converted to the active form, e.g., by enzymes within the cell. In certain embodiments senolytic prodrug is designed so that the prodrug is converted to an active form by enzymes that are expressed at a higher level in senescent cells than in non-senescent cells.

In various embodiments senolytic agents described herein that may alter at least one signaling pathway include, but are not limited to, an agent that inhibits an activity of at least one of the target proteins within the pathway. In certain embodiments the senolytic agent may be a specific inhibitor of one or more BCL-2 anti-apoptotic protein family members wherein the inhibitor inhibits at least BCL-xL (e.g., a Bcl-2/Bcl-xL/Bcl-w inhibitor; a selective Bcl-xL inhibitor; a Bcl-xL/Bcl-w inhibitor), an Akt kinase specific inhibitor, and/or an MDM2 inhibitor. In certain embodiments, molecules such as quercetin (and analogs thereof), enzastaurin, and dasatinib are excluded and are not compounds used in the methods and compositions described herein. In other particular embodiments, methods described herein involve the use of at least two senolytic agents wherein at least one senolytic agent and a second senolytic agent are each different and independently alter either one or both of a survival signaling pathway and an inflammatory pathway in a senescent cell.

Small Senolytic Molecules

Senolytic agents that may be used in the methods described herein include, but are not limited to, small organic molecules. In various embodiments the small organic molecules (also called small molecules or small molecule compounds herein) typically have molecular weights less than about 10⁵ daltons, or less than about 10⁴ daltons, or less than about 10³ daltons. In certain embodiments, a small molecule senolytic agent does not violate the following criteria more than once: (1) no more than 5 hydrogen bond donors (the total number of nitrogen-hydrogen and oxygen-hydrogen bonds); (2) not more than 10 hydrogen bond acceptors (all nitrogen or oxygen atoms); (3) a molecular mass less than 500 daltons; (4) an octanol-water partition coefficient[5] log P not greater than 5.

MDM2 Inhibitors

In certain embodiments, the senolytic agent(s) used in the methods described herein may comprise an MDM2 inhibitor. In certain embodiments an MDM2 (murine double minute 2) inhibitor that may be used in the methods described herein may be a small organic molecule compound that belongs to any one of the following classes of compounds, for example, an imidazoline compound (e.g., cis-imidazoline compound), a spiro-oxindole compound, a benzodiazepine compound, a piperidinone compound, a tryptamine compound, CGM097 ((S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one), and related analogs. In certain embodiments, the MDM2 inhibitor is also capable of binding to and inhibiting an activity of MDMX (murine double minute X, which is also known as HDMX in humans). The human homolog of MDM2 is called HDM2 (human double minute 2) in the art. Therefore, when the subject to which the senolytic inhibitors are administered in accordance with the methods described herein is a human subject, the compounds described herein as MDM2 inhibitors may also inhibit binding of HDM2 to one or more of its ligands.

MDM2 is described in the art as an E3 ubiquitin ligase that can promote tumor formation by targeting tumor suppressor proteins, such as p53, for proteasomal degradation through the 26S proteasome (see, e.g., Haupt et al. (1997) Nature 387: 296-299; Honda et al. (1997) FEBS Lett. 420: 25-27; Kubbutat et al. (1997) Nature, 387: 299-303; and the like). MDM2 also affects p53 by directly binding to the N-terminal end of p53, which inhibits the transcriptional activation function of p53 (see, e.g., Momand et al. (1992) Cell 69: 1237-1245; Oliner et al. (1993) Nature 362: 857-860). Mdm2 is in turn regulated by p53see, e.g., Lahav (2008) Exp. Med. Biol. 641: 28-38). MDM2 activities, include, inter alia, activity as a ubiquitin ligase E3 toward itself and ARRB1, facilitation of nuclear export of p53; promotion of proteasome-dependent ubiquitin-independent degradation of retinoblastoma RB1 protein, inhibition of DAXX-mediated apoptosis by inducing its ubiquitination and degradation, action as a component of TRIM28/KAP1-MDM2-p53 complex involved in stabilizing p53; component of TRIM28/KAP1-ERBB4-MDM2 complex that links growth factor and DNA damage response pathways; mediation of ubiquitination and subsequent proteasome degradation of DYRK2 in the nucleus; ubiquitination of IGF1R and SNAI1 and promotion of these moieties to proteasomal degradation. MDM2 has also been reported to induce mono-ubiquitination of the transcription factor FOXO4 (see, e.g., Brenkman et al. (2008) PLOS One, 3(7): e2819). In various embodiments the MDM2 inhibitors described herein may disrupt the interaction between MDM2 and any one or more of the aforementioned cellular components.

In one embodiment, a senolytic agent useful in the methods described herein comprises an imidazoline (e.g., a cis-imidazoline). Cis-imidazoline compounds include, but are not limited to, those called nutlins in the art. Similar to other MDM2 inhibitors described herein, nutlins are cis-imidazoline small molecule inhibitors of the interaction between MDM2 and p53 (see, e.g., Vassilev et al. (2004) Science 303(5659): 844-48). Illustrative, but non-limiting examples of cis-imidazolines compounds that may be used in the methods described herein are described in U.S. Pat. Nos. 6,734,302; 6,617,346; and 7,705,007 and in U.S. Patent Pub. Nos: 2005/0282803, 2007/0129416, and 2013/0225603. In certain embodiments, the methods described herein comprise use of a nutlin compound called Nutlin-1, or a nutlin compound called Nutlin-2, or a Nutlin compound called Nutlin-3 (see, e.g., CAS Registry Nos. 675576-98-4 and 548472-68-0). The active enantiomer of Nutlin-3 (4-[[4S,5R)-4,5-bis(4-chlorophenyl)-4,5-dihydro-2-[4-methoxy-2-(−1-methylethoxy)phenyl]-1H-imidazol-1-yl]carbonyl]-2-piperazinone) is called Nutlin-3a in the art. In certain embodiments, the methods described herein comprise use of Nutlin-3a for selectively killing senescent cells.

Nutlin-3 is described in the art as a nongenotoxic activator of the p53 pathway, and the activation of p53 is controlled by the murine double minute 2 (MDM2) gene. The MDM2 protein is an E3 ubiquitin ligase and controls p53 half-life by way of ubiquitin-dependent degradation. Nutlin-3a has been investigated in pre-clinical studies (e.g., with respect to pediatric cancers) and clinical trials for treatment of certain cancers (e.g., retinoblastoma). To date in vitro and pre-clinical studies with Nutlin-3 have suggested that the compound has variable biological effects on the function of cells exposed to the compound. For example, Nutlin-3 reportedly increases the degree of apoptosis of cancer cells in hematological malignancies including B-cell malignancies (see, e.g., Zauli et al., (2011) Clin. Cancer Res. 17: 762-770 and references cited therein) and in combination with other chemotherapeutic drugs, such as dasatinib, the cytotoxic effect appears synergistic (see, e.g., Zauli et al., supra).

In certain embodiments the imidazoline compound comprises a compound having the structure:

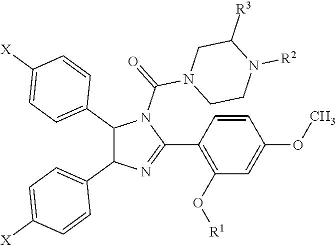

or a pharmaceutically acceptable salt thereof; where X is halide (e.g., Cl, Fl, etc.); R¹ is alkyl (e.g., C1-C12 alkyl), R² is —H or heteroalkyl, and R³ is —H or ═O. In certain embodiments, as noted above, the imidazoline compound is selected from the group consisting of nutlin-1, nutlin-2, and nutlin-3. In certain embodiments the imidazoline compound comprises a 4-[[(4S,5R)-4,5-bis(4-chlorophenyl)-4,5-dihydro-2-[4-methoxy-2-(1-methyle-thoxy)phenyl]-1H-imidazol-1-yl]carbonyl]-2-piperazinone or a pharmaceutically acceptable salt thereof.

In certain embodiments the imidazoline compound comprises a compound having the structure:

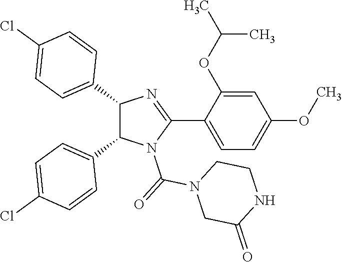 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

or a pharmaceutically acceptable salt thereof.

Another illustrative senolytic cis-imidazoline compound useful for the methods described herein RG-7112 (Roche) (CAS No: 939981-39-2; IUPAC name: ((4S,5R)-2-(4-(tert-butyl)-2-ethoxyphenyl)-4,5-bis(4-chlorophenyl)-4,5-di-methyl-4,5-dihydro-1H-imidazol-1-yl)(4-(3-(methylsulfonyl)propyl)piperazin-1-yl)methanone (see, e.g., U.S. Pat. No. 7,851,626; Tovar et al. (2013) Cancer Res. 72: 2587-2597).

In another illustrative embodiment, an MDM2 inhibitor useful in the methods described herein is a cis-imidazoline compound called RG7338 (Roche) (IPUAC Name: 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoic acid) (CAS 1229705-06-9); Ding et al. (2013) J. Med. Chem. 56(14): 5979-5983; Zhao et al. (2013) J. Med. Chem. 56(13): 5553-5561). Yet another illustrative nutlin compound is R05503781 (also known as idasanutlin). Other potent cis-imidazoline small molecule compounds include dihydroimidazothiazole compounds (e.g., DS-3032b; Daiichi Sankyo) (see, also, Miyazaki et al. (2013) Bioorg. Med. Chem. Lett. 23(3): 728-732; Miyazaki et al. (2012) Bioorg. Med. Chem. Lett. 22(20): 6338-6342; PCT Pub. No: WO 2009/151069).

In certain embodiments, a cis-imidazoline compound that may be used in the methods described herein is a dihydroimidazothiazole compound.

In certain embodiments, the MDM2 small molecule inhibitor comprises a spiro-oxindole compound (see, e.g., compounds described in Ding et al. (2005) J. Am. Chem. Soc. 127: 10130-10131; Shangary et al. (2008) Proc. Natl. Acad. Sci. USA, 105: 3933-3938; Shangary et al. (2008) Mol. Cancer Ther. 7: 1533-1542; Hardcastle et al., (2005) Bioorg. Med. Chem. Lett. 15: 1515-1520; Hardcastle et al. (2006) J. Med. Chem. 49(21): 6209-6221; Watson et al. (2011) Bioorg. Med. Chem. Lett. 21(19): 5916-5919, which are incorporated herein by reference for the spiro-oxindole compounds described therein). Other examples of spiro-oxindole compounds that are MDM2 inhibitors are called in the art MI-63, MI-126; MI-122, MI-142, MI-147, MI-18, MI-219, MI-220, MI-221, and MI-773. Another specific spiro-oxindole compound is 3-(4-chlorophenyl)-3-((1-(hydroxymethyl)cyclopropyl)methoxy)-2-(4-nitrobe-nzyl)isoindolin-1-one. Another compound is called MI888 (see, e.g., Zhao et al. (2013) J. Med. Chem. 56(13): 5553-5561; PCT Pub. No: WO 2012/065022).

In certain embodiments the MDM2 small molecule inhibitor that may be used in the methods described herein comprises a benzodiazepinedione (see, e.g., Grasberger et al. (2005) J. Med. Chem. 48: 909-912; Parks et al. (2005) Bioorg. Med. Chem. Lett. 15: 765-770; Raboisson et al. (2005) Bioorg. Med. Chem. Lett. 15: 1857-1861; Koblish et al. (2006) Mol. Cancer Ther. 5: 160-169). Benzodiazepinedione compounds that may be used in the methods described herein include, but are not limited to, 1,4-benzodiazepin-2,5-dione compounds. Examples of benzodiazepinedione compounds include 5-[(3S)-3-(4-chlorophenyl)-4-[(R)-1-(4-chlorophenyl)ethyl]-2,5-dioxo-7-ph-enyl-1,4-diazepin-1-yl]valeric acid and 5-[(3S)-7-(2-bromophenyl)-3-(4-chlorophenyl)-4-[(R)-1-(4-chlorophenyl)eth-yl]-2,5-dioxo-1,4-diazepin-1-yl]valeric acid (see, e.g., Raboisson et al., supra). Other benzodiazepinedione compounds are called in the art TDP521252 (IUPAC Name: 5-[(3S)-3-(4-chlorophenyl)-4-[(1R)-1-(4-chlorophenyl)ethyl]-7-ethynyl-2,5-dioxo-3H-1,4-benzodiazepin-1-yl]pentanoic acid) and TDP665759 (IUPAC Name: (3S)-4-[(1R)-1-(2-amino-4-chlorophenyl)ethyl]-3-(4-chlorophenyl)-7-iodo-1-[3-(4-methylpiperazin-1-yl)propyl]-3H-1,4-benzodiazepine-2,5-dione) (see, e.g., Parks et al., supra; Koblish et al., supra).

In certain embodiments, the MDM2 small molecule inhibitor comprises a terphenyl (see, e.g., Yin et al. (2005) Angew Chem. Int. Ed. Engl. 44: 2704-2707; Chen et al. (2005) Mol. Cancer Ther. 4: 1019-1025). In certain embodiments, the MDM2 inhibitor that may be used in the methods described herein comprises a quilinol (see, e.g., Lu et al. (2006) J. Med. Chem. 49: 3759-3762). In certain embodiments, the MDM2 inhibitor comprises a chalcone (see, e.g., Stoll et al. (2001) Biochm. 40: 336-344). In certain embodiments, the MDM2 inhibitor is a sulfonamide (e.g., NSC279287) (see, e.g., Galatin et al. (2004) J. Med. Chem. 47: 4163-4165).

In certain embodiments, a compound that may be used in the methods described herein comprises a tryptamine, such as serdemetan (JNJ-26854165; chemical name: N1-(2-(1H-indol-3-yl)ethyl)-N4-(pyridine-4-yl)benzene-1,4-diamine; CAS No. 881202-45-5) (Johnson & Johnson, New Brunswick, N.J.). Serdemetan is a tryptamine derivative that activates p53 and acts as a HDM2 ubiquitin ligase antagonist (see, e.g., Chargari et al. (2011) Cancer Lett. 312(2): 209-218; Kojima et al. (2010) Mol. Cancer Ther. 9: 2545-2557; Yuan et al. (2011) J. Hematol. Oncol. 4:16).

In certain embodiments, MDM2 small molecule inhibitors that may be used in the methods described herein include, but are not limited to those described in Rew et al. (2012) J. Med. Chem. 55: 4936-4954; Gonzalez-Lopez de Turiso et al. (2013) J. Med. Chem. 56: 4053-4070; Sun et al. (2014) J. Med. Chem. 57: 1454-1472).

In certain embodiments, the MDM2 inhibitor comprises a piperidinone compound. An example of a potent MDM2 piperidinone inhibitor is AM-8553 ({(3R,5R,6S)-5-(3-Chlorophenyl)-6-(4-chlorophenyl)-1-[(2S,3 S)-2-hydroxy-3-pentanyl]-3-methyl-2-oxo-3-piperidinyl}acetic acid; CAS No. 1352064-70-0) (Amgen, Thousand Oaks, Calif.).

In certain embodiments, an MDM2 inhibitor that may be used in the methods described herein comprises a piperidine (see, e.g., PCT Publ. No: WO 2011/046771). In certain embodiments, an MDM2 inhibitor that may be used in the methods described herein comprises an imidazole-indole compound (see, e.g., PCT Pub. No: WO 2008/119741).

Examples of compounds that bind to MDM2 and to MDMX and that may be used in the methods described herein also include, but are not limited to, RO-2443 and RO-5963 ((Z)-2-(4-((6-Chloro-7-methyl-1H-indol-3-yl)methylene)-2,5-dioxoimidazoli-din-1-yl)-2-(3,4-difluorophenyl)-N-(1,3-dihydroxypropan-2-yl)acetamide) (see, e.g., Graves et al. (2012) Proc. Natl. Acad. Sci. USA, 109: 11788-11793; Zhao et al. (2013) BioDiscovery, supra).

In another illustrative, but non-limiting embodiment, an MDM2 inhibitor referred to in the art as CGM097 may be used in the methods described herein.

Inhibitors of BCL-2 Anti-Apoptotic Family of Proteins

In certain embodiments, the senolytic agent used in the methods described herein may be an inhibitor of one or more proteins in the BCL-2 family. In certain embodiments, at least one senolytic agent is selected from an inhibitor of one or more BCL-2 anti-apoptotic protein family members wherein the inhibitor inhibits at least BCL-xL. Typically, inhibitors of BCL-2 anti-apoptotic family of proteins alter at least a cell survival pathway. Apoptosis activation may occur via an extrinsic pathway triggered by the activation of cell surface death receptors or an intrinsic pathway triggered by developmental cues and diverse intracellular stresses. This intrinsic pathway, also known as the stress pathway or mitochondrial pathway, is primarily regulated by the BCL-2 family, a class of key regulators of caspase activation consisting of anti-apoptotic (pro-survival) proteins having BH1-BH4 domains (BCL-2 (i.e., the BCL-2 protein member of the BCL-2 anti-apoptotic protein family), BCL-xL, BCL-w, A1, MCL-1, and BCL-B); pro-apoptotic proteins having BH1, BH2, and BH3 domains (BAX, BAK, and BOK); and pro-apoptotic BH3-only proteins (BIK, BAD, BID, BIM, BMF, HRK, NOXA, and PUMA) (see, e.g., Cory et al., Nature Reviews Cancer 2:647-56 (2002); Cory et al., Cancer Cell 8:5-6 (2005); Adams et al., Oncogene 26:1324-1337 (2007)). BCL-2 anti-apoptotic proteins block activation of pro-apoptotic multi-domain proteins BAX and BAK (see, e.g., Adams et al., Oncogene 26:1324-37 (2007)). While the exact mechanism of apoptosis regulation is unknown, it is hypothesized that BH3-only proteins unleashed by intracellular stress signals bind to anti-apoptotic BCL-2 like proteins via a BH3 “ligand” to a “receptor” BH3 binding groove formed by BH1-3 regions on anti-apoptotic proteins, thereby neutralizing the anti-apoptotic proteins (see, e.g., Letai et al. (2002) Cancer Cell, 2: 183-192; Adams et al., Oncogene, supra). BAX and BAK can then form oligomers in mitochondrial membranes, leading to membrane permeabilization, release of cytochrome C, caspase activation, and ultimately apoptosis (see, e.g., Adams et al., Oncogene, supra).

As used herein, in various embodiments a BCL-2 family member that is inhibited by the agents described herein is a pro-survival (anti-apoptotic) family member. The senolytic agents used in the methods described herein inhibit one or more functions of the BCL-2 anti-apoptotic protein, BCL-xL (which may also be written herein and in the art as Bcl-xL, BCL-XL, Bcl-xl, or Bcl-XL). In certain embodiments, in addition to inhibiting BCL-xL function, the inhibitor may also interact with and/or inhibit one or more functions of BCL-2 (e.g., BCL-xL/BCL-2 inhibitors). In certain embodiments, senolytic agents used in the methods described herein are classified as inhibitors of each of BCL-xL and BCL-w (i.e., BCL-xL/BCL-w inhibitors). In certain embodiments, senolytic agents used in the methods described herein that inhibit BCL-xL may also interact with and inhibit one or more functions of each of BCL-2 (i.e., the BCL-2 protein) and BCL-w (i.e., BCL-xL/BCL-2/BCL-w inhibitors), thereby causing selective killing of senescent cells. In certain embodiments, a BCL-2 anti-apoptotic protein inhibitor interferes with the interaction between the BCL-2 anti-apoptotic protein family member (which includes at least BCL-xL) and one or more ligands or receptors to which the BCL-2 anti-apoptotic protein family member would bind in the absence of the inhibitor. In certain embodiments, an inhibitor of one or more BCL-2 anti-apoptotic protein family members wherein the inhibitor inhibits at least BCL-xL specifically binds only to one or more of BCL-xL, BCL-2, BCL-w and not to other Bcl-2 anti-apoptotic Bcl-2 family members, such as Mcl-1 and BCL2A1.

In certain embodiments, the senolytic agent used in the methods described herein is a BCL-xL selective inhibitor and inhibits one or more functions of BCL-xL. Such senolytic agents that are BCL-xL selective inhibitors do not inhibit the function of one or more other BCL-2 anti-apoptotic proteins in a biologically or statistically significant manner BCL-xL may also be called BCL2L1, BCL2-like 1, BCLX, BCL2L, BCLxL, or BCL-X herein and in the art. In one embodiment, BCL-xL selective inhibitors alter (e.g., reduce, inhibit, decrease, suppress) one or more functions of BCL-xL but do not significantly inhibit one or more functions of other proteins in the BCL-2 anti-apoptotic protein family (e.g., BCL-2 or BCL-w). In certain embodiments, a BCL-xL selective inhibitor interferes with the interaction between BCL-xL and one or more ligands or receptors to which BCL-xL would bind in the absence of the inhibitor. In certain particular embodiments, a senolytic agent that inhibits one or more of the functions of BCL-xL selectively binds to human BCL-xL but not to other proteins in the BCL-2 family, which effects selective killing of senescent cells.

BCL-xL is an anti-apoptotic member of the BCL-2 protein family. BCL-xL also plays an important role in the crosstalk between autophagy and apoptosis (see, e.g., Zhou et al. (2011) FEBS J. 278: 403-413). BCL-xL also appears to play a role in bioenergetic metabolism, including mitochondrial ATP production, Ca²⁺ fluxes, and protein acetylation, as well as on several other cellular and organismal processes such as mitosis, platelet aggregation, and synaptic efficiency (see, e.g., Michels et al. (2013) Int. J. Cell Biol., Vol. 2013, Article ID 705294). In certain embodiments, the BCL-xL inhibitors described herein may disrupt the interaction between BCL-xL and any one or more of the aforementioned BH3-only proteins to promote apoptosis in cells.

In certain embodiments, a BCL-xL inhibitor used in the methods described herein is a selective inhibitor, meaning, that it preferentially binds to BCL-xL over other anti-apoptotic BCL2 family members (e.g., BCL-2, MCL-1, BCL-w, BCL-b, and BFL-1/A1). In certain embodiments, a BCL-XL selective inhibitor exhibits at least a 5-fold, 10-fold, 50-fold, 100-fold, 1000-fold, 10000-fold, 20000-fold, or 30000-fold selectivity for binding a BCL-XL protein or nucleic acid over a BCL-2 protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over a MCL-1 protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over a BCL-w protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-XL protein or nucleic acid over a BCL-B protein or nucleic acid. In certain embodiments, a BCL-XL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over an A1 protein or nucleic acid. As described herein, in certain embodiments, an inhibitor of one or more BCL-2 anti-apoptotic protein family members wherein the inhibitor inhibits at least BCL-xL (e.g., a BCL-xL selective inhibitor) has no detectable binding to MCL-1 or to BCL2A1.

Methods for measuring binding affinity of a BCL-xL inhibitor for BCL-2 family proteins are known in the art. By way of example, binding affinity of a BCL-xL inhibitor may be determined using a competition fluorescence polarization assay in which a fluorescent BAK BH3 domain peptide is incubated with BCL-xL protein (or other BCL-2 family protein) in the presence or absence of increasing concentrations of the BCL-XL inhibitor as previously described (see, e.g., U.S. Patent Pubb: 2014/0005190; Park et al. (2013) Cancer Res. 73: 5485-5496; Wang et al. (2000) Proc. Natl. Acad. Sci. USA, 97: 7124-7129; Zhang et al. (2002) Anal. Biochem. 307: 70-75; Bruncko et al. (2007) J. Med. Chem. 50: 641-662). Percent inhibition may be determined by the equation: 1−[(mP value of well-negative control)/range)]×100%. Inhibitory constant (K₁) value is determined by the formula: Kᵢ=[I]₅₀/([L]₅₀/K_{d}+[P]₀/K_{d}+1) as described in Bruncko et al. (2007) J. Med. Chem. 50: 641-662 (see, also, Wang (1995) FEBS Lett. 360: 111-114).

In certain embodiments senolytic agents (e.g., BCL-xL selective inhibitors, BCL-xL/BCL-2 inhibitors, BCL-xL/BCL-2/BCL-w inhibitors, BCL-xL/BCL-w inhibitors) used in the methods described herein.

In certain embodiments, the BCL-xL inhibitor comprises a small molecule compound that is a benzothiazole-hydrazone compound, an aminopyridine compound, a benzimidazole compound, a tetrahydroquinoline compound, a phenoxyl compound, and/or related analogs.

In one embodiment, a BCL-xL selective inhibitor useful for the methods described herein comprises a benzothiazole-hydrazone inhibitor. Benzothiazole-hydrazone compounds include, but are not limited to, WEHI-539 (5-[3-[4-(aminomethyl)phenoxy]propyl]-2-[(8E)-8-(1,3-benzothiazol-2-ylhyd-razinylidene)-6,7-dihydro-5H-naphthalen-2-yl]-1,3-thiazole-4-carboxylic acid), a BH3 peptide mimetic that selectively targets BCL-xL (see, e.g., Lessene et al. (2103) Nat. Chem. Biol., 9: 390-397).

In certain embodiments, the BCL-xL selective inhibitor comprises an aminopyridine compound. One illustrative, but non-limiting, aminopyridine compound that may be used as a selective BCL-xL inhibitor comprises BXI-61 (3-[(9-amino-7-ethoxyacridin-3-yl)diazenyl]pyridine-2,6-diamine) (see, e.g., Park et al. (2013) Cancer Res. 73: 5485-5496, and U.S. Patent Pub. No: 2009/0118135).

In still other embodiments, a BCL-xL selective inhibitor that may be used in the methods described herein is a benzimidazole compound. One example of a benzimidazole compound that may be used as a selective BCL-XL inhibitor is BXI-72 (2′-(4-Hydroxyphenyl)-5-(4-methyl-1-piperazinyl)-2,5′-bi(1H-benzimidazole-) trihydrochloride) (see, e.g., Park et al. (2013) supra). In certain embodiments, the methods described herein utilize BXI-72 for selectively killing senescent cells.

In certain embodiments the BCL-xL selective inhibitor used in the methods described herein comprises a tetrahydroquinoline compound (see, e.g., U.S. Patent Publ. No. 2014/0005190). Examples of tetrahydroquinoline compounds that can be used as selective BCL-xL inhibitors are shown in Table 1 of U.S. Patent Publ. No. 2014/0005190 and described therein which is incorporated herein by reference for the compounds described therein. Other inhibitors described therein may inhibit other BCL-2 family members (e.g., BCL-2) in addition to BCL-xL.

In certain embodiments, a BCL-xL selective inhibitor used in the methods described herein comprises a phenoxyl compound. One non-limiting example of a phenoxyl compound that can be used as a selective BCL-xL inhibitor is 2[[3-(2,3-dichlorophenoxy) propyl]amino]ethanol (2,3-DCPE) (see, e.g., Wu et al. (2004) Cancer Res. 64: 1110-1113).

In still another embodiment, an inhibitor of a Bcl-2 anti-apoptotic family member that inhibits at least BCL-xL that can be used in the methods described herein is described in U.S. Pat. No. 8,232,273. In one illustrative embodiment, the inhibitor comprises a BCL-xL selective inhibitor called A-1155463 (see, e.g., Tao et al. (2014) ACS Med. Chem. Lett. 5(10): 1088-1093).

In certain embodiments senolytic agent(s) useful in the methods described herein inhibits other BCL-2 anti-apoptotic family members in addition to BCL-xL. For example, in certain embodiments the methods described herein comprise use of BCL-xL/BCL-2 inhibitors, and/or BCL-xL/BCL-2/BCL-w inhibitors, and/or BCL-xL/BCL-w inhibitors and analogs thereof. In certain embodiments, the inhibitors include compounds that inhibit BCL-2 and BCL-xL, which inhibitors may also inhibit BCL-w. Examples of these inhibitors include, but are not limited to ABT-263 (4-[4-[[2-(4-chlorophenyl)-5,5-dimethylcyclohexen-1-yl]methyl]piperazin-1--yl]-N-[4-[[(2R)-4-morpholin-4-yl-1-phenylsulfanylbutan-2-yl]amino]-3-(tri-fluoromethylsulfonyl)phenyl]sulfonylbenzamide or IUPAC, (R)-4-(4-((4′-chloro-4,4-dimethyl-3,4,5,6-tetrahydro-[1,1′-biphenyl]-2-yl-) methyl)piperazin-1-yl)-N-((4-((4-morpholino-1-(phenylthio)butan-2-yl)amin-o)-3-((trifluoromethyl)sulfonyl)phenyl)sulfonyl)benzamide) (see, e.g., Park et al. (2008) J. Med. Chem. 51: 6902-6915; Tse et al. (2008) Cancer Res. 68: 3421-3428; PCT Pub. No. WO 2009/155386; U.S. Pat. Nos. 7,390,799; 7,709,467; 7,906,505; 8,624,027; and the like), and ABT-737 (4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfo-nyl]benzamide, Benzamide, 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfon-yl]- or 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfony-lbenzamide) (see, e.g., Oltersdorf et al., (2005) Nature, 435: 677-681; U.S. Pat. Nos. 7,973,161, and 7,642,260). In certain embodiments, the BCL-2 anti-apoptotic protein inhibitor is a quinazoline sulfonamide compound (see, e.g., Sleebs et al. (2011) J. Med. Chem. 54:1914-1926). In certain embodiments, the BCL-2 anti-apoptotic protein inhibitor is a small molecule compound as described in Zhou et al. (2012) J. Med. Chem. 55: 4664-4682 (see, e.g., Compound 21 (R)-4-(4-chlorophenyl)-3-(3-(4-(4-(4-((4-(dimethylamino)-1-(phenylthio)bu-tan-2-yl)amino)-3-nitrophenylsulfonamido)phenyl)piperazin-1-yl)phenyl)-5-e-thyl-1-methyl-1H-pyrrole-2-carboxylic acid), and Zhou et al. (2012) J. Med. Chem. 55: 6149-6161 (see, e.g., Compound 14 (R)-5-(4-Chlorophenyl)-4-(3-(4-(4-(4-((4-(dimethylamino)-1-(phenylthio)bu-tan-2-yl)amino)-3-nitrophenylsulfonamido)phenyl)piperazin-1-yl)phenyl)-1-e-thyl-2-methyl-1H-pyrrole-3-carboxylic acid; Compound 15 (R)-5-(4-Chlorophenyl)-4-(3-(4-(4-(4-(4-(dimethylamino)-1-(phenylthio)but-an-2-yl)amino)-3-nitrophenylsulfonamido)phenyl)piperazin-1-yl)phenyl)-1-is-opropyl-2-methyl-1H-pyrrole-3-carboxylic acid). In certain embodiments, the BCL-2 anti-apoptotic protein inhibitor is a BCL-2/BCL-xL inhibitor such as BM-1074 (see, e.g., Aguilar et al. (2013) J. Med. Chem. 56: 3048-3067); BM-957 (see, e.g., Chen et al. (2012) J. Med. Chem. 55: 8502-8514); BM-1197 (see, e.g., Bai et al. (2014) PLoS One, 9(6):e99404; U.S. Patent Pub. No. 2014/0199234); N-acylsulfonamide compounds (see, e.g., PCT Pub. Nos. WO 2002/024636, WO 2005/049593, and WO 2005/049594, and U.S. Pat. Nos. 7,767,684, and 7,906,505). In certain embodiments, the BCL-2 anti-apoptotic protein inhibitor is a small molecule macrocyclic compound (see, e.g., PCT Pub. No. WO 2006/127364, and U.S. Pat. No. 7,777,076). In certain embodiments, the BCL-2 anti-apoptotic protein inhibitor comprises an isoxazolidine compound (see, e.g., PCT Pub. No. WO 2008/060569; U.S. Pat. Nos. 7,851,637, and 7,842,815).

In certain embodiments, the senolytic agent comprises a compound that is an inhibitor of Bcl-2, Bcl-w, and Bcl-xL, such as ABT-263 or ABT-737. In certain specific embodiments, the senolytic agent comprises a compound or a pharmaceutically acceptable salt, stereoisomer, tautomer, or prodrug thereof as illustrated below, which depicts the structure of ABT-263. ABT-263 is also known as Navitoclax in the art.

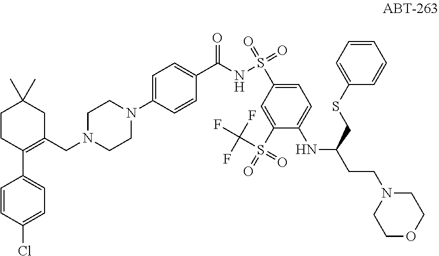 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

### Akt Kinase Inhibitors

In certain embodiments the senolytic agent comprises an Akt Kinase inhibitor. In some embodiments, the senolytic agent comprises a compound that selectively inhibits Akt1, Akt2, and/or Akt3, relative to other protein kinases.

Akt inhibitors (also known as Akt kinase inhibitors or AKT kinase inhibitors) can be divided into six major classes based on their mechanisms of action (see, e.g., Bhutani et al. (2013) Infectious Agents and Cancer, 8: 49). Akt is also called protein kinase B (PKB) in the art. The first class contains ATP competitive inhibitors of Akt and includes compounds such as CCT128930 and GDC-0068, which inhibit Akt2 and Akt1. This category also includes the pan-Akt kinase inhibitors such as GSK2110183 (afuresertib), GSK690693, and AT7867. The second class contains lipid-based Akt inhibitors that act by inhibiting the generation of PIP3 by PI3K. This mechanism is employed by phosphatidylinositol analogs such as Calbiochem Akt Inhibitors I, II and III or other PI3K inhibitors such as PX-866. This category also includes compounds such as Perifosine (KRX-0401) (Aeterna Zentaris/Keryx). The third class contains a group of compounds called pseudosubstrate inhibitors. These include compounds such as AKTide-2 T and FOXO3 hybrid. The fourth class consists of allosteric inhibitors of AKT kinase domain, and include compounds such as MK-2206 (8-[4-(1-aminocyclobutyl)phenyl]-9-phenyl-2H-[1,2,4]triazolo[3,4-f][1,6]n-aphthyridin-3-one; dihydrochloride) (Merck & Co.) (see, e.g., U.S. Pat. No. 7,576,209, which is incorporated herein by reference for the compounds described therein). The fifth class consists of antibodies and includes molecules such as GST-anti-Akt1-MTS. The last class comprises compounds that interact with the PH domain of Akt, and includes Triciribine and PX-316. Other compounds described in the art that act as AKT inhibitors include, for example, GSK-2141795 (GlaxoSmithKline), VQD-002, miltefosine, AZD5363, GDC-0068, and API-1.

In on illustrative, but non-limiting embodiment, the senolytic agent is a compound is an Akt kinase inhibitor having the structure as shown below (also called MK-2206 herein and in the art), 8-[4-(1-aminocyclobutyl)phenyl]-9-phenyl-2H-[1,2,4]triazolo[3,4-f][1,6]na-phthyridin-3-one) or a pharmaceutically acceptable salt, stereoisomer, tautomer, or prodrug thereof. The dihydrochloride salt is shown.

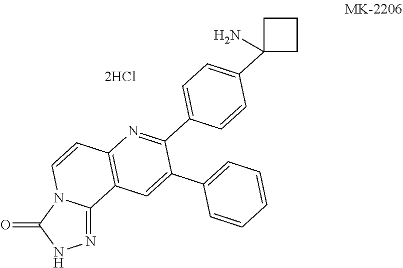 [SMILES: NC1(c2ccc(-c3nc4ccn5c(=O)[nH]nc5c4cc3-c3ccccc3)cc2)CCC1]

Combinations of Senolytic Agents.

In certain embodiments, at least one senolytic agent may be administered with at least one other senolytic agent. In certain embodiments the two or more senolytic agents act additively or synergistically to selectively kill senescent cells. In particular embodiments, the methods described herein utilize a senolytic agent that alters either a cell survival signaling pathway or an inflammatory pathway or alters both the cell survival signaling pathway and the inflammatory pathway in a senescent cell. In certain embodiments, the methods described herein comprise use of at least two senolytic agents wherein at least one senolytic agent and a second senolytic agent are each different and independently alter either one or both of a survival signaling pathway and an inflammatory pathway in a senescent cell. For convenience, when two or more senolytic agents are described herein as being used in combination, one senolytic agent can be called a first senolytic agent, another senolytic agent can be called the second senolytic agent, etc. In other certain embodiments, the methods described herein comprise administering at least three senolytic agents (a first senolytic agent, second senolytic agent, and third senolytic agent). The adjectives, first, second, third, and such, in this context are used for convenience only and are not to be construed as describing order or administration, preference, or level of senolytic activity or other parameter unless expressly described otherwise. In particular embodiments, when two or more senolytic agents are used in the methods described herein, each senolytic agent is a small molecule.

In certain embodiments, the methods described herein comprise administering at least three senolytic agents (a first senolytic agent, second senolytic agent, and third senolytic agent). In certain embodiments, use of at least two senolytic agents results in significantly increased killing of senescent cells compared with use of each senolytic agent alone. In other particular embodiments, use of at least two senolytic agents results in significant killing of senescent cells compared with use of each senolytic agent alone and which effect may be additive or synergistic. In certain embodiments, at least two senolytic agents are each different and selected from (1) an inhibitor of one or more BCL-2 anti-apoptotic protein family members wherein the inhibitor inhibits at least BCL-xL; (for example, a Bcl-2/Bcl-xL/Bcl-w inhibitor, a Bcl-2/Bcl-xL inhibitor, a selective Bcl-xL inhibitor, or a Bcl-xL/Bcl-w inhibitor); an Akt kinase specific inhibitor; a MDM2 inhibitor. In one particular embodiment, when at least one senolytic agent administered to a subject is an inhibitor of one or more BCL-2 anti-apoptotic protein family members wherein the inhibitor inhibits at least BCL-XL (e.g., a Bcl-2/Bcl-xL/Bcl-w inhibitor, a Bcl-2/Bcl-xL inhibitor, a selective Bcl-xL inhibitor, or a Bcl-xL/Bcl-w inhibitor), a second senolytic agent is administered. In other certain embodiments, one of the two senolytic agents is an inhibitor of one or more BCL-2 anti-apoptotic protein family members wherein the inhibitor inhibits at least BCL-xL and the second senolytic agent is an MDM2 inhibitor. In yet still more particular embodiments, when at least one senolytic agent administered to a subject is a selective Bcl-xL inhibitor, a second senolytic agent is administered. In still more particular embodiments, when at least one senolytic agent administered to a subject is an MDM2 inhibitor, a second senolytic agent is administered. In still more particular embodiments, when at least one senolytic agent administered to a subject is an Akt kinase inhibitor, a second senolytic agent is administered. In even more particular embodiments, the inhibitor of one or more BCL-2 anti-apoptotic protein family members wherein the inhibitor inhibits at least BCL-xL is used alone or in combination with another senolytic agent that is also an inhibitor of one or more BCL-2 anti-apoptotic protein family members wherein the inhibitor inhibits at least BCL-xL or is a different senolytic agent as described herein. In particular embodiments, an inhibitor of one or more BCL-2 anti-apoptotic protein family members wherein the inhibitor inhibits at least BCL-xL is combined with an inhibitor of Akt kinase. By way of non-limiting example, the Bcl-2/Bcl-xL/Bcl-w inhibitor ABT-263 may be used in combination with an Akt kinase inhibitor (e.g., MK2206).

In still other particular embodiments, an MDM2 inhibitor that is a senolytic agent is used in combination with at least one additional senolytic agent in the methods described herein. The additional senolytic agent (which may be referred to for convenience as a second senolytic agent) may be another MDM2 inhibitor or may be a senolytic agent that is not a MDM2 inhibitor. In one embodiment, an inhibitor of a Bcl-2 anti-apoptotic family member that inhibits at least Bcl-xL is used in combination with an AKT inhibitor. In a more specific embodiment, the inhibitor of a Bcl-2 anti-apoptotic family member is ABT-263, ABT-737, or WEHI-539 and the AKT inhibitor is MK-2206.

In other certain embodiments, the methods described herein comprise administering at least three senolytic agents (a first senolytic agent, second senolytic agent, and third senolytic agent).

mTOR, NFκB, and PI3-k Pathway Inhibitors

In certain embodiments a compound that may be used together with a senolytic agent described herein in the methods described herein (e.g., for selectively killing senescent cells) may comprise a compound that inhibits one or more of mTOR, NFκB, and PI3-k pathways. Thus, for example, in the methods described herein, administration of a senolytic agent (e.g., for selectively killing senescent cells) may comprise administering to the subject at issue at least one senolytic agent and an inhibitor of one or more of mTOR, NFκB, and PI3-k pathways. Inhibitors of these pathways are known in the art.

Examples of mTOR inhibitors include, but are not limited to, sirolimus, temsirolimus, everolimus, ridaforolimus, 32-deoxorapamycin, zotarolimus, PP242, INK128, PP30, Torinl, Ku-0063794, WAY-600, WYE-687 and WYE-354. Inhibitors of an NF.kappa.B pathway include, for example, NF.kappa.B activity abrogation through TPCA-1 (an IKK2 inhibitor); BAY 11-7082 (an IKK inhibitor poorly selective for IKK1 and IKK2); and MLN4924 (an NEDD8 activating enzyme (NAE)-inhibitor), and MG132.

Examples of inhibitors of PI3-k that may also inhibit mTOR and/or AKT pathways include, but are not limited to, perifosine (KRX-0401), idelalisib, PX-866, IPI-145, BAY 80-6946, BEZ235, RP6530, TGR 1201, SF1126, INK1117, GDC-0941, BKM120, XL147 (SAR245408), XL765 (SAR245409), Palomid 529, GSK1059615, GSK690693, ZSTK474, PWT33597, IC87114, TG100-115, CAL263, RP6503, PI-103, GNE-477, CUDC-907, AEZS-136, BYL719, BKM120, GDC-0980, GDC-0032, and MK2206.

Small Organic Molecules—Salts and General Synthesis Procedures.

Various small organic molecules contemplated herein as senolytic agents include physiologically acceptable salts (i.e., pharmaceutically acceptable salts), hydrates, solvates, polymorphs, metabolites, and prodrugs of the senolytic agents. Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

In certain embodiments the senolytic agents that comprise small organic molecules may generally be used as the free acid or free base. Alternatively, the compounds may be used in the form of acid or base addition salts. Acid addition salts of the free base amino compounds may be prepared according to methods well known in the art, and may be formed from organic and inorganic acids. Suitable organic acids include, but are not limited to, maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, malonic, and benzenesulfonic acids. Suitable inorganic acids include, but are not limited to hydrochloric, hydrobromic, sulfuric, phosphoric, and nitric acids. Base addition salts of the free acid compounds of the compounds described herein may also be prepared by methods well known in the art, and may be formed from organic and inorganic bases. Additional salts include those in which the counterion is a cation. Suitable inorganic bases included (but are not limited to) the hydroxide or other salt of sodium, potassium, lithium, ammonium, calcium, barium, magnesium, iron, zinc, copper, manganese, aluminum, and the like, and organic bases such as substituted ammonium salts (for example, dibenzylammonium, benzylammonium, 2-hydroxyethylammonium). Further salts include those in which the counterion is an anion, such as adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate. Thus, the term “pharmaceutically acceptable salt” of compounds described herein is intended to encompass any and all pharmaceutically suitable salt forms.

Compounds may sometimes be depicted as an anionic species. One of ordinary skill in the art will recognize that the compounds can exist with an equimolar ratio of cation. For instance, the compounds described herein can exist in the fully protonated form, or in the form of a salt such as sodium, potassium, ammonium or in combination with any inorganic base as described above. When more than one anionic species is depicted, each anionic species may independently exist as either the protonated species or as the salt species. In some specific embodiments, the compounds described herein exist as the sodium salt. In other specific embodiments, the compounds described herein exist as the potassium salt.

In certain embodiments, some of the crystalline forms of any compound described herein may exist as polymorphs, which are also included and contemplated by the present disclosure. I n addition, some of the compounds may form solvates with water or other organic solvents. Often crystallizations produce a solvate of the disclosed compounds. As used herein, the term “solvate” refers to an aggregate that comprises one or more molecules of any of the disclosed compounds with one or more molecules of solvent. The solvent may be water, in which case the solvate may be a hydrate. Alternatively, the solvent may be an organic solvent. Thus, in certain embodiments, the senolytic agents described herein may exist as a hydrate, including a monohydrate, dihydrate, hemihydrate, sesquihydrate, trihydrate, tetrahydrate and the like, as well as the corresponding solvated forms. In certain embodiments the compounds may be true solvates, while in other instances, the compounds may simply retain adventitious water or may be a mixture of water and some adventitious solvent.

In general, the compounds used in the methods described herein may be made according to organic synthesis techniques known to those skilled in this art, starting from commercially available chemicals and/or from compounds described in the chemical literature. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002. Methods known to one of ordinary skill in the art may be identified through various reference books and databases. Suitable reference books and treatises detail the synthesis of reactants useful in the preparation of compounds described herein, or provide references to articles that describe the preparation.

Polypeptides, Antibodies, and Nucleic Acids

In certain embodiments, a senolytic agent used in the methods described herein can comprise a polypeptide, peptide, antibody, antigen-binding fragment, peptibody, recombinant viral vector, or a nucleic acid. In certain embodiments, the senolytic agent comprises an antisense oligonucleotide, siRNA, shRNA, or a peptide. For example, senolytic agents such as polypeptides, antibodies, nucleic acids, and the like, include, for example, MDM2 inhibitors, BCL-2 family inhibitors, or Akt kinase inhibitors. In other embodiments, polypeptides, peptides, antibodies (including antigen-binding fragments thereof) that specifically bind to a ligand or target protein of a small molecule senolytic agent described herein, may be used in assays and methods for characterizing or monitoring the use of the small molecule senolytic agent.

A polynucleotide or oligonucleotide that specifically hybridizes to a portion of mRNA that encodes a target protein (e.g., Bcl-xL, Bcl-2, Bcl-w, MDM2, Akt) of a cell that is a senescent cell or that is a cell in a disease microenvironment may induce the cell to senescence by aging, a biologically damaging (i.e., cell damaging) medical therapy, or an environmental insult. In other embodiments, the target protein may be a ligand, or protein either downstream or upstream in a cell survival pathway or inflammatory pathway or apoptotic pathway. Polynucleotides and oligonucleotides may be complementary to at least a portion of a nucleotide sequence encoding a target polypeptide (e.g., a short interfering nucleic acid, an antisense polynucleotide, a ribozyme, or a peptide nucleic acid) and that may be used to alter gene and/or protein expression. These polynucleotides that specifically bind to or hybridize to nucleic acid molecules that encode a target polypeptide may be prepared using the nucleotide sequences available in the art. In another embodiment, nucleic acid molecules such as aptamers that are not sequence-specific may also be used to alter gene and/or protein expression.

Antisense polynucleotides bind in a sequence-specific manner to nucleic acids such as mRNA or DNA. Identification of oligonucleotides and ribozymes for use as antisense agents and identification of DNA encoding the target gene for targeted delivery involve methods well known in the art. For example, the desirable properties, lengths, and other characteristics of such oligonucleotides are well known. Antisense technology can be used to control gene expression through interference with binding of polymerases, transcription factors, or other regulatory molecules (see, e.g., Gee et al., In Huber and Carr, Molecular and Immunologic Approaches, Futura Publishing Co. (Mt. Kisco, N.Y.; 1994)).

Short interfering RNAs may be used for modulating (decreasing or inhibiting) the expression of a gene encoding a target polypeptide of interest. Small nucleic acid molecules, such as short interfering RNA (siRNA), micro-RNA (miRNA), and short hairpin RNA (shRNA) molecules may be used according to the methods described herein to modulate the expression of a target protein. A siRNA polynucleotide preferably comprises a double-stranded RNA (dsRNA) but may comprise a single-stranded RNA (see, e.g., Martinez et al. (2002) Cell 110: 563-574). An siRNA polynucleotide may comprise other naturally occurring, recombinant, or synthetic single-stranded or double-stranded polymers of nucleotides (ribonucleotides or deoxyribonucleotides or a combination of both) and/or nucleotide analogues as provided herein and known and used by persons skilled in the art.

The term “siRNA” refers to a double-stranded interfering RNA unless otherwise noted. Typically, an siRNA is a double-stranded nucleic acid molecule comprising two nucleotide strands, each strand having about 19 to about 28 nucleotides (i.e., about 19, 20, 21, 22, 23, 24, 25, 26, 27, or 28 nucleotides). In certain embodiments, each strand is 19, 20, 21, 22, or 23 nucleotides. In other particular embodiments, the siRNA comprises two nucleotide strands, each strand having about 15, 16, 17, or 18 nucleotides. In other certain embodiments, one strand of the double stranded siRNA is at least two nucleotides longer, for example, one strand may have a two-base overhang (such as TT) at one end, usually the 3′ terminal end.

Short hairpin interfering RNA molecules comprise both the sense and antisense strands of an interfering RNA in a stem-loop or hairpin structure (e.g., a shRNA). An shRNA may be expressed from a DNA vector in which the DNA oligonucleotides encoding a sense interfering RNA strand are linked to the DNA oligonucleotides encoding the reverse complementary antisense interfering RNA strand by a short spacer. If needed, 3′ terminal T's and nucleotides forming restriction sites may be added. The resulting RNA transcript folds back onto itself to form a stem-loop structure.

In addition to siRNA molecules, other interfering RNA and RNA-like molecules can interact with RISC and silence gene expression, such as short hairpin RNAs (shRNAs), single-stranded siRNAs, microRNAs (miRNAs), and dicer-substrate 27-mer duplexes. Such RNA-like molecules may contain one or more chemically modified nucleotides, one or more non-nucleotides, one or more deoxyribonucleotides, and/or one or more non-phosphodiester linkages. RNA or RNA-like molecules that can interact with RISC and participate in RISC-related changes in gene expression may be referred to herein as “interfering RNAs” or “interfering RNA molecules.” Single-stranded interfering RNA in certain instances effects mRNA silencing, but less efficiently than double-stranded RNA.

A person skilled in the art will also recognize that RNA molecules, such as siRNA, miRNA, shRNA, may be chemically modified to confer increased stability against nuclease degradation while retaining the capability to bind to the target nucleic acids that may be present in cells. The RNA may be modified at any position of the molecule so long as the modified RNA binds to the target sequence of interest and resists enzymatic degradation. Modifications in the siRNA may be in the nucleotide base, the ribose, or the phosphate. By way of example, the T position of ribose can be modified, which modification can be accomplished using any one of a number of different methods routinely practiced in the art. An RNA may be chemically modified by the addition of a halide such as fluoro. Other chemical moieties that have been used to modify RNA molecules include methyl, methoxyethyl, and propyl groups (see, e.g., U.S. Pat. No. 8,675,704).

In certain embodiments, the polynucleotide or oligonucleotide (e.g., including a shRNA) may be delivered by a recombinant vector into which the polynucleotide or oligonucleotide of interest has been incorporated. In other embodiments, the recombinant viral vector may be a recombinant expression vector into which a polynucleotide sequence that encodes an antibody, an antigen-binding fragment, polypeptide or peptide that inhibits a protein in a cell survival pathway or an inflammatory pathway, including the proteins described herein such as Bcl-xL, Bcl-2, Bcl-w, MDM2, and Akt is inserted such that the encoding sequence is operatively linked with one or more regulatory control sequences to drive expression of the polypeptide, antibody, an antigen-binding fragment, or peptide. The recombinant vector or the recombinant expression vector may be a viral recombinant vector or a viral recombinant expression vector. Illustrative viral vectors include, without limitation, a lentiviral vector genome, poxvirus vector genome, vaccinia virus vector genome, adenovirus vector genome, adenovirus-associated virus vector genome, herpes virus vector genome, and alpha virus vector genome. Viral vectors may be live, attenuated, replication conditional or replication deficient, and typically is a non-pathogenic (defective), replication competent viral vector. Procedures and techniques for designing and producing such viral vectors are well known to and routinely practiced by persons skilled in the art.

In certain embodiments a senolytic agent that may be used in the methods described herein comprises an antisense oligonucleotide. By way of non-limiting example, BCL-xL specific antisense oligonucleotides that have been previously described may be used in the methods described herein (see, e.g., PCT Publ. No. WO 00/66724; Xu et al., (2001) Intl. J. Cancer 94: 268-274; Olie et al. (2002) J. Invest. Dermatol. 118: 505-512; and Wacheck et al. (2003) Br. J. Cancer, 89: 1352-1357).

In certain embodiments, a senolytic agent that may be used in the methods described herein comprises a peptide. By way of example and in certain embodiments, a BCL-xL selective peptide inhibitor is a BH3 peptide mimetic. Examples of BCL-xL selective BH3 peptide mimetics include those previously described (see, e.g., Kutzki et al., (2002) J. Am. Chem. Soc. 124: 11838-11839; Yin et al. (2004) Bioorg. Med. Chem. Lett. 22: 1375-1379; Matsumura et al. (2010) FASEB J. 7: 2201).

In certain embodiments, a senolytic agent useful in the methods described herein does not include a polynucleotide, or a fragment thereof, that encodes the exonuclease, EXO1, or a vector (including a viral vector) that comprises a polynucleotide that encodes the EXO1 enzyme (i.e., a polynucleotide encoding an EXO1 enzyme, a fragment of the polynucleotide, or a vector containing such a polynucleotide is excluded). In certain embodiments a senolytic agent useful in the methods described herein also does not include the EXO1 enzyme polypeptide (i.e., the EXO1 enzyme is excluded) or biologically active peptide or polypeptide fragment thereof. In certain embodiments such molecules are not inhibitors of one or both of a cell signaling pathway, such as an inflammatory pathway or a cell survival pathway; instead EXO1 encodes a 5′-3′ exonuclease that degrades capping defective telomeres (see, e.g., PCT Pub No: WO 2006/018632).

A senolytic useful in the methods described herein may comprise a polypeptide that is an antibody, or antigen-binding fragment. In certain embodiments the antigen-binding fragment may be an F(ab′)₂, Fab, Fab′, Fv, or Fd and can also include a peptide or polypeptide that comprises at least one complementary determining region (CDR). The antibody may be an internalizing antibody or antigen-binding fragment that is internalized by the senescent cell via interaction with a target protein.

Binding properties of an antibody to its cognate antigen, may generally be determined and assessed using methods that may be readily performed by those having ordinary skill in the art (see, e.g., Harlow et al., Antibodies: A Laboratory Manual, Cold Spring Harbor Laboratory (1988)). As used herein, an antibody is said to be “immunospecific,” “specific for” or to “specifically bind” to an antigen if it reacts at a detectable level with the polypeptide Affinities of antibodies and antigen binding fragments thereof can be readily determined using conventional techniques, for example, those described by Scatchard et al. (1949) Ann. N.Y. Acad. Sci. USA 51: 660, and by surface plasmon resonance (SPR; BIAcore™, Biosensor, Piscataway, N.J.).

In various embodiments the antibodies may be polyclonal or monoclonal. A variable region or one or more complementarity determining regions (CDRs) may be identified and isolated from antigen-binding fragment or peptide libraries. An antibody, or antigen-binding fragment, may be recombinantly engineered and/or recombinantly produced. An antibody may belong to any immunoglobulin class, for example IgG, IgE, IgM, IgD, or IgA and may be obtained from or derived from an animal, for example, fowl (e.g., chicken) and mammals, which include but are not limited to a mouse, rat, hamster, rabbit, or other rodent, a cow, horse, sheep, goat, camel, human, or other primate. For use in human subjects, antibodies and antigen-binding fragments are typically human, humanized, or chimeric to reduce an immunogenic response by the subject to non-human peptides and polypeptide sequences.

In various embodiments the antibody may be a monoclonal antibody that is a human antibody, humanized antibody, chimeric antibody, bispecific antibody, or an antigen-binding fragment (e.g., F(ab′)₂, Fab, Fab′, Fv, and Fd) prepared or derived therefrom. An antigen-binding fragment may also be any synthetic or genetically engineered protein (see, e.g., Hayden et al. (1997) Curr. Opin. Immunol. 9: 201-212; Coloma et al. (1997) Nat. Biotechnol. 15: 159-163; U.S. Pat. No. 5,910,573; Holliger et al., (1997) Cancer Immunol. Immunother. 45: 128-130; Drakeman et al. (1997) Exp. Opin. Investig. Drugs, 6: 1169-1178; Koelemij et al. (1999) J. Immunother. 22: 514-524; Marvin et al. (2005) Acta Pharmacol. Sin. 26:649-658; Das et al., (2005) Meth. Mol. Med. 109: 329-346; PCT Pub Nos: PCT/US91/08694 and PCT/US91/04666) and from phage- or yeast-display libraries (see, e.g., Scott et al. (1990) Science, 249: 386-390; Devlin et al., (1990) Science 249: 404-406; Cwirla et al. (1997) Science, 276: 1696-1699; U.S. Pat. Nos. 5,223,409, 5,733,731, 5,498,530, 5,432,018, 5,338,665, and 5,922,545, PCT Pub Nos: WO 96/40987 and WO 98/15833). A peptide that is a minimal recognition unit or a CDR (i.e., any one or more of the three CDRs present in a heavy chain variable region and/or one or more of the three CDRs present in a light chain variable region) may be identified by computer modeling techniques, which can be used for comparing and predicting a peptide sequence that will specifically bind to a target protein of interest (see, e.g., Bradley et al., (2005) Science 309: 1868; Schueler-Furman et al. (2005) Science 310: 638). Useful strategies for designing humanized antibodies are described in the art (see, e.g., Jones et al., (1986) Nature 321: 522-525; Riechmann et al. (1988) Nature, 332: 323-327; Padlan et al., (1995) FASEB 9: 133-139; Chothia et al. (1989) Nature, 342: 377-383).

Senolytic Viruses.

In certain embodiments the senolytic agents include engineered senolytic viruses that specifically kill senescent cells. Such viruses are described, inter alia, in U.S. Patent Pub. No: US 2015/0064137 A1.

The foregoing senolytic agents described above are illustrative and non-limiting. Using the teachings provided herein numerous other senolytic agents useful in the methods described herein will be known to those of skill in the art.

Pathologies Associated with Elevated Senescent Cells.

As explained above, in various embodiments, the methods of identifying elevated levels of senescent cells in a subject (e.g., by determining the levels of one or more of an eicosanoid, an eicosanoid precursor, leukotriene A4 (LTA4), leukotriene B4 (LTB4), PGD2, and 5-HETE) are use in the context of a differential diagnosis of, and/or to identify a treatment modality for, a pathology characterized by elevated levels of senescent cells. In certain embodiments Dihomo-15d-PGJ2 is one or more most important eicosanoid markers. In certain embodiments methods of treatment are provided that involve administering one or more senolytic agents to a subject identified as halving elevated levels of one or more of the markers described herein (and by implication one or more pathologies characterized by elevated levels of senescent cells). In certain embodiments methods are provided for evaluating a treatment regimen that involves administration of one or more senolytic agents to a subject having elevated levels of senescent cells (e.g., a subject with a pathology characterized by elevated levels of senescent cells).

Such pathologies (characterized by elevated levels of senescent cells) include, inter alia, various diseases, or disorders related to, associated with, or caused by cellular senescence, including age-related diseases and disorders in a subject. A senescence-associated disease or disorder may also be called herein a senescent cell-associated disease or disorder. Senescence-associated diseases and disorders include, for example, cardiovascular diseases and disorders, inflammatory diseases and disorders, autoimmune diseases and disorders, pulmonary diseases and disorders, eye diseases and disorders, metabolic diseases and disorders, neurological diseases and disorders (e.g., neurodegenerative diseases and disorders), age-related diseases and disorders induced by senescence, skin conditions, dermatological diseases and disorders, and transplant related diseases and disorders. A prominent feature of aging is a gradual loss of function, or degeneration that occurs at the molecular, cellular, tissue, and organismal levels. Age-related degeneration gives rise to well-recognized pathologies, such as sarcopenia, atherosclerosis and heart failure, osteoporosis, pulmonary insufficiency, renal failure, neurodegeneration (including macular degeneration, Alzheimer's disease, and Parkinson's disease), and many others. Although different mammalian species vary in their susceptibilities to specific age-related pathologies, collectively, age-related pathologies generally rise with approximately exponential kinetics beginning at about the mid-point of the species-specific life span (e.g., 50-60 years of age for humans) (see, e.g., Campisi (2013) Annu. Rev. Physiol. 75: 685-705; Naylor et al. (2013) Clin. Pharmacol. Ther. 93: 105-116).

Examples of senescence-associated conditions, disorders, or diseases that may be treated using the methods described herein, and/or whose treatment may be evaluated by the methods described herein and/or that may be diagnosed using the methods described herein include, but are not limited to, cognitive diseases (e.g., mild cognitive impairment (MCI), Alzheimer's disease and other dementias; Huntington's disease), cardiovascular disease (e.g., atherosclerosis, cardiac diastolic dysfunction, aortic aneurysm, angina, arrhythmia, cardiomyopathy, congestive heart failure, coronary artery disease, myocardial infarction, endocarditis, hypertension, carotid artery disease, peripheral vascular diseases, cardiac stress resistance, cardiac fibrosis), metabolic diseases and disorders (e.g., obesity, diabetes, metabolic syndrome); motor function diseases and disorders (e.g., Parkinson's disease, motor neuron dysfunction (MND), Huntington's disease); cerebrovascular disease; emphysema; osteoarthritis; benign prostatic hypertrophy; cancer progression and metastasis; pulmonary diseases (e.g., idiopathic pulmonary fibrosis, chronic obstructive pulmonary disease (COPD), emphysema, obstructive bronchiolitis, asthma), inflammatory/autoimmune diseases and disorders (e.g., osteoarthritis, eczema, psoriasis, osteoporosis, mucositis, transplantation related diseases and disorders); ophthalmic diseases or disorders (e.g., age-related macular degeneration, cataracts, glaucoma, vision loss, presbyopia); diabetic ulcer; metastasis, a chemotherapeutic side effect; a radiotherapy side effect; a side effect of highly active antiretroviral therapy (HAART); aging-related diseases and disorders (e.g., kyphosis, renal dysfunction, frailty, hair loss, hearing loss, muscle fatigue, skin conditions, sarcopenia, and herniated intervertebral disc, a and other age-related diseases that are induced by senescence (e.g., diseases/disorders resulting from irradiation, chemotherapy, HAART smoking tobacco, eating a high fat/high sugar diet, and environmental factors); wound healing; skin nevi; fibrotic diseases and disorders (e.g., cystic fibrosis, renal fibrosis, liver fibrosis, pulmonary fibrosis, oral submucous fibrosis, cardiac fibrosis, and pancreatic fibrosis). In certain embodiments, any one or more of the diseases or disorders described above or herein may be excluded.

In certain embodiments the pathology comprises osteoarthritis, osteoporosis, sarcopenia, idiopathic pulmonary fibrosis, chronic obstructive pulmonary disease (COPD), cancer progression, or atherosclerosis.

### EXAMPLES

The following examples are offered to illustrate, but not to limit the claimed invention.

### Example 1

### Senescent Cells are a Source of Bioactive Lipids During Lung Fibrosis

### Introduction

Cellular senescence is a multifaceted response to cellular stress or damage that results in both a permanent mitotic arrest and secretion of a number of factors with potent biological activities (Campisi & d'Adda di Fagagna (2007) Nat. Rev. Mol. Cell Biol. 8: 729-740; Coppe et al. (2008) PLoS Biol. 6: 2853-2868; Acosta et al. (2008) Cell, 133: 1006-1018; Kuilman et al. (2010) Genes Dev. 24, 2463-2479). This senescence-associated secretory phenotype, or SASP, has largely been studied in the context of secreted proteins, many of which have been shown to have key roles in several processes including development, aging, wound healing, arthrosclerosis, cancer progression and fibrosis (Coppe et al. (2008) PLoS Biol. 6: 2853-2868; Munoz-Espin et al. Cell, 155: 1104-1118; Baker et al. (2016) Nature 530: 184-189; Demaria et al. (2014) Dev. Cell, 31: 722-733; Childs et al. (2016) Science 354: 472-477; Krizhanovsky et al. (2008) Cell, 134: 657-667). By comparison, secretion of lipids and other molecules has been understudied in the context of senescence. Here we show that senescent cells activate biosynthesis of eicosanoids—signaling lipids with potent biological effects, and this activity both promotes the inflammatory part of the SASP and reinforces mitotic arrest. Furthermore, in a model of damaged-induced lung fibrosis, elimination of senescent cells both lowers levels of specific eicosanoids and attenuates the fibrotic response. These data highlight a new and potentially important aspect of cellular senescence, and provide a novel therapeutic target for degenerative conditions driven by senescent cells, such as pulmonary fibrosis.

Cellular senescence is a multifaceted stress response that results in an essentially permanent mitotic arrest, coupled with several phenotypic changes including cellular hypertrophy, nuclear and epigenetic rearrangements, and metabolic alterations (Campisi & d'Adda di Fagagna (2007) Nat. Rev. Mol. Cell Biol. 8: 729-740; Kuilman et al. (2010) Genes Dev. 24, 2463-2479; Wiley & Campisi (2016) Cell Metab. 23: 1013-1021). Senescent cells drive pathologies associated with aging and other degenerative conditions in part by secreting a myriad of biologically active molecules including inflammatory cytokines and chemokines, matrix metalloproteinases, and growth factors that have potent local—and potentially systemic—effects on tissues (Coppe et al. (2008) PLoS Biol. 6: 2853-2868; Acosta et al. (2008) Cell, 133: 1006-1018). Ultimately the accumulation of senescent cells limits both lifespan and healthspan of mice during natural aging⁶. Thus far, this senescence-associated secretory phenotype (SASP) has been studied almost exclusively in the context of secreted proteins. Here we show that senescent cells also synthesize a large number of eicosanoids—a class of biologically active signaling lipids that are often secreted into the extracellular milieu and promote diverse responses such as inflammation, fever, vasoconstriction and vasodilation, pain, hair loss, asthma, and fibrosis (Funk (2001) Science 294:1871-1875; Soberman & Christmas (2003) J. Clin. Invest. 111: 1107-1113).

Recent evidence indicates that both senescence and the SASP are under metabolic control, suggesting mechanisms for potential intervention (Wiley & Campisi (2016) Cell Metab. 23: 1013-1021). In order to better understand the metabolic changes that occur during cellular senescence, we extracted intracellular lipids and aqueous metabolites from proliferating (PRO, 10% FBS), quiescent (QUI, 0.2% FBS), as well as ionizing radiation (IR)-induced senescent (SEN(IR)) IMR-90 fibroblasts cultured in either medium (10% FBS or 0.2% FBS), and measured their relative abundances by mass spectrometry. Thus, we were able to control for differences attributable to both growth state (QUI vs PRO vs SEN(IR)) and culture medium (0.2% vs 10%). From the lipid profiles, we determined that certain subsets of lipids showed strong elevation or decline with senescence. These included ceramides, saturated fatty acids, and retinoic acid. Most notable, however, were striking elevations in relative abundances of eicosanoids: a class of potent signaling lipids derived from 20-carbon fatty acids, most notably arachidonic acid. The most abundant of these eicosanoids was 1a,1b-dihomo-15-deoxy-delta12,14-prostaglandin J2 (dihomo-15d-PGJ2), but dihomo versions of prostaglandin D2 (PGD2) and prostaglandin E2 (PGE2) were also detected (FIG. 1, panel A). Additionally, we observed increases in specific leukotrienes during senescence, notably leukotrienes A4 (LTA4) and B4 (LTB4), as well as the related lipoxygenase product, 5-HETE (FIG. 1, panel A). Additionally, the eicosanoid precursors arachidonic acid (AA), eicosapentanoic acid (EPA), and dihomo-gamma-linoleic acid (DGLA) were elevated in senescent cells (FIG. 1, panel B). Adrenic acid, a product of the elongation of AA and precursor of the dihomo prostaglandins, was similarly elevated during senescence (FIG. 1, panel B). Thus, both eicosanoids and their precursors are elevated with senescence.

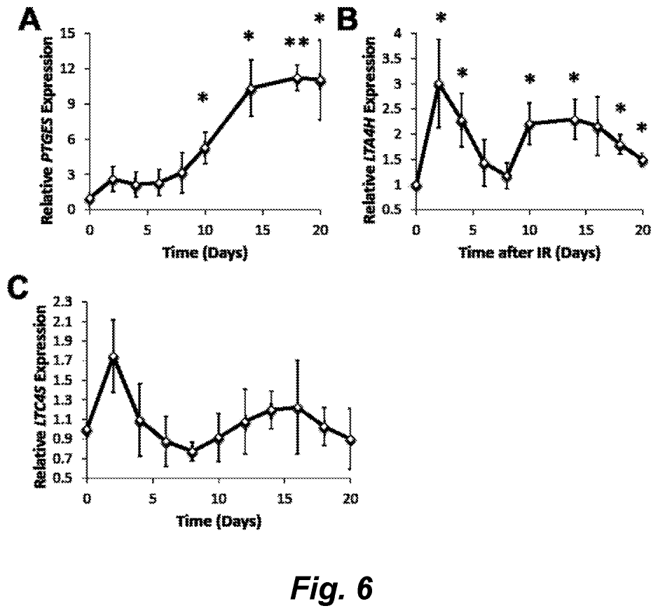

Arachidonic acid is released from the plasma membrane by the activities of phospholipases, particularly cytosolic phospholipase 2 (cPLA2) (Lin et al. (1993) Cell, 72: 269-278). cPLA2 is phosphorylated at serine 505 by p38MAPK (Kramer et al. (1996) J. Biol. Chem. 271: 27723-27729), a kinase activated during senescence (Freund et al. (2011) EMBO J. 30: 1536-1548). Following induction of senescence, we observed activation (phosphorylation) of p38MAPK and a commensurate increase in cPLA2 phosphorylation (FIG. 1, panel C), as previously reported (Lin et al. (1993) Cell, 72: 269-278; Kramer et al. (1996) J. Biol. Chem. 271: 27723-27729). Additionally, we measured mRNA expression of eicosanoid synthesis pathway genes in senescent cells by quantitative PCR. Senescent cells elevated expression of several eicosanoid synthesis genes (FIG. 1, panel D), including prostaglandin synthases PTGS2 (COX-2), PTGES, PTGDS, but not PTGIS or TBXAS. Leukotriene synthesis mRNAs including ALOX5 (5-LO), ALOX15, ALOX5AP, LTC4S, and LTA4H were similarly elevated. Of the three fatty acid elongases (ELOVLs) that synthesize adrenic acid, only ELOVL4 was elevated at senescence (FIG. 1, panel D). Notably, time courses of gene expression revealed that while prostaglandin synthase expression increased exponentially in senescent cells (FIG. 1, panel E, FIG. 6, panel A), leukotriene synthase expression was biphasic—with a large increase 2 days after Irradiation and a later, lower response 10-20 days following IR (FIG. 1, panel F, FIG. 6, panels B, C).

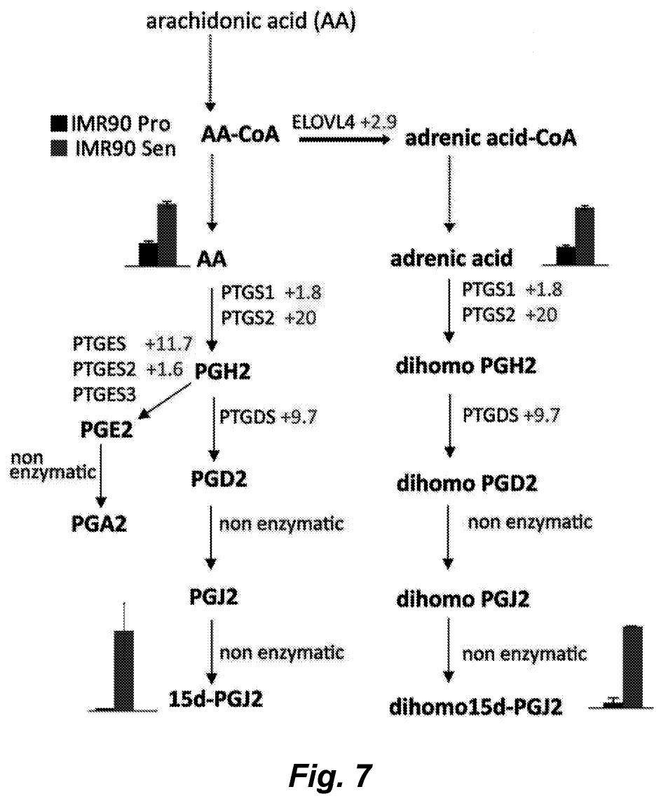

Since dihomo-15d-PGJ2 was highly elevated and abundant (˜1.4 μM) at senescence—and was until now a theoretical compound—we confirmed its identity. While dihomo-prostaglandin standards are unavailable, the identity of dihomo-15d-PGJ2 was determined using commercially available 15d-PGJ2 (Cayman). Dihomo-15d-PGJ2 differs from 15-PGJ2 by the addition of a C₂H₄ attachment resulting in a mass shift of 28 Da (from 315 to 343m/z). In addition to exact mass, the MS/MS fragmentation pattern of dihomo-15d-PGJ2 produces fragments identical to 15d-PGJ2 with the addition of a 28 Da mass shift, confirming the presence of two additional CH2 groups (FIG. 1, panels G-I) (Harkewicz et al. (2007) J. Biol. Chem. 282: 2899-2910). This, in combination with the evidence for increased cPLA2 activity and arachidonic acid, ELOVL4 expression and adrenic acid, PTGDS expression and dihomo-PGD2 (FIG. 7), confirm that dihomo-15d-PGJ2 is the most likely metabolite detected by our analysis.

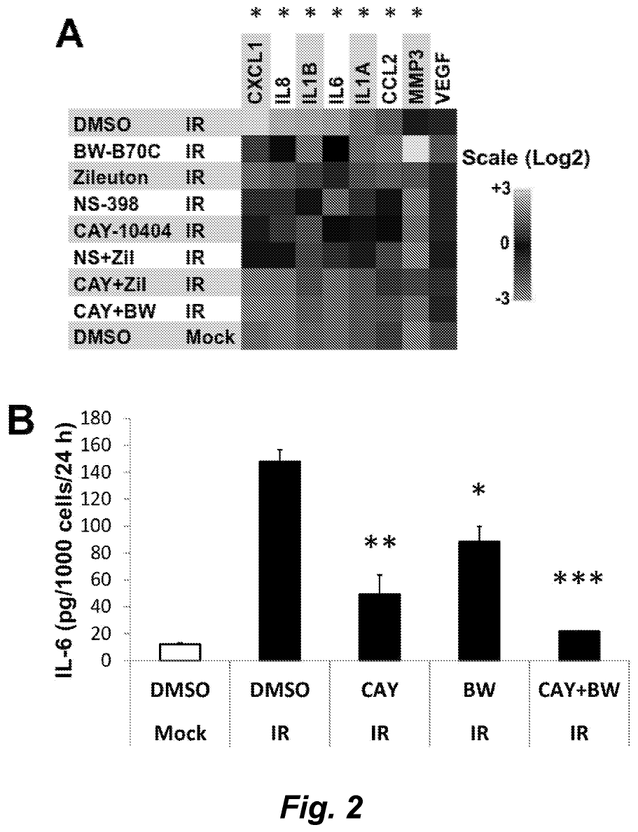

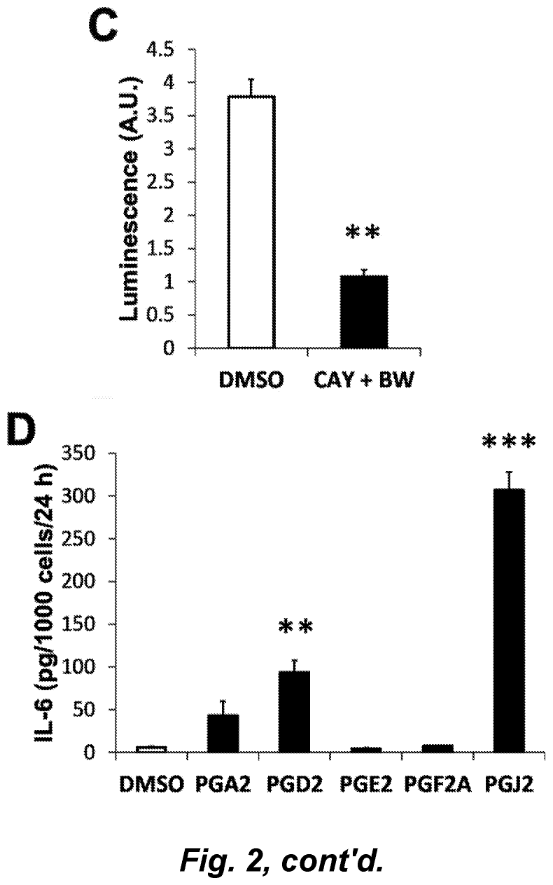

Most prostaglandins and leukotrienes are secreted; therefore, these eicosanoids might be considered a novel lipid component of the SASP. We also considered the possibility that, since many eicosanoids promote growth and inflammation (Ricciotti & FitzGerald (2011) Arterioscler. Thromb. Vasc. Biol. 31: 986-1000; Castellone et al. (2005) Science, 310: 1504-1510; Dennis & Norris (2015) Nat. Rev. Immunol. 15: 511-523), senescence-associated eicosanoid biosynthesis might promote the inflammatory SASP. We therefore inhibited prostaglandin synthesis with either CAY-10404 (CAY) or NS-293 (NS) (PTGS2/COX-2-specific inhibitors), leukotriene synthesis with BW-B70C (BW) or zileuton (Zil) (ALOX5 inhibitors), or synthesis of both prostaglandins and leukotrienes using a combination of COX-2 and ALOX5 inhibitors in senescent (IR) cells—and measured mRNA levels of several SASP factors (FIG. 2, panel A). While ALOX5 inhibitors only slightly decreased levels of most SASP factors, COX-2 inhibitors decreased SASP factor levels more strongly, and the combination of COX-2 and ALOX5 inhibitors lowered SASP levels to near non-senescent (DMSO+Mock) levels. By comparison, VEGF, a pro-angiogenic component of the SASP, was not decreased by inhibitor treatment (FIG. 2, panel A). We also measured IL-6 secretion in senescent cells following a subset of treatments (CAY, BW, or CAY+BW). CAY lowered IL-6 secretion in senescent cells by ˜70%, while BW-B70C lowered it by ˜50%, and the combination of the inhibitors was additive, lowering IL-6 secretion to near baseline levels (FIG. 2, panel B). This combination of inhibitors also lowered the transactivation activity of NF-κB (FIG. 2, panel C), a major transcription factor that promotes the proinflammatory SASP in senescent cells (Freund et al. (2011) EMBO J. 30: 1536-1548).

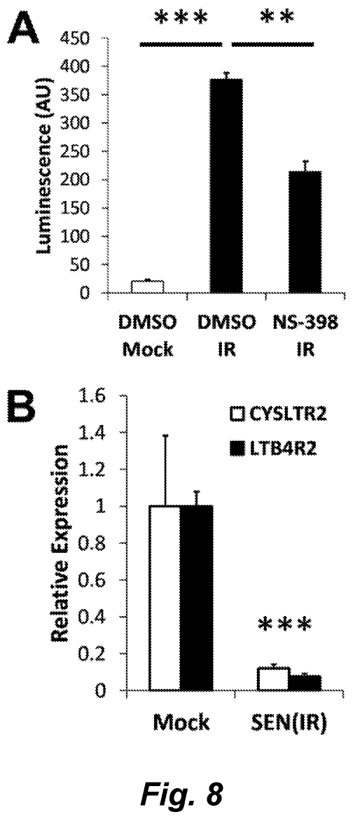

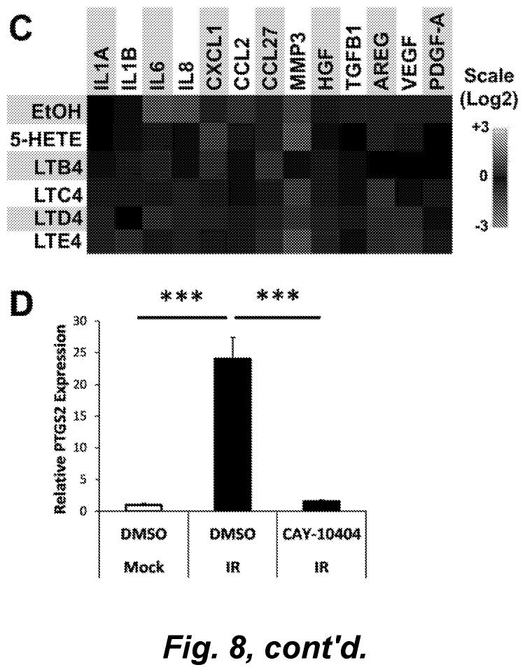

Since dihomo-15d-PGJ2 was the most abundant prostaglandin in our analysis (FIG. 1A), and 15d-PGJ2 is an endogenous ligand for PPARγ (Forman et al. (1995) Cell, 83: 803-812), we also assessed PPARγ transactivation by luciferase reporter assay (FIG. 8, panel A). PPARγ activity was elevated ˜18-fold in senescent cells, and this activation was reduced ˜50% in response to NS-398, demonstrating PPARγ activation in senescent cells is partially prostaglandin-dependent.

Since inhibition of COX-2 strongly attenuated much of the SASP (FIG. 2, panels A-B), we sought to determine which prostaglandins might be responsible for SASP maintenance. We therefore treated non-senescent cells with 10 μM of PGA2, PGD2, PGE2, PGF2α, or PGJ2. Of these, PGA2, PGD2, PGJ2 elevated IL-6 secretion in response to treatment, with PGJ2 displaying the highest induction (FIG. 2, panel D). We also measured expression of additional SASP factors by qPCR (FIG. 2, panels E-F). All prostaglandins elevated mRNA levels of IL1A and IL1B, as well as MMP3. Senescent cells lost leukotriene receptor expression (FIG. 8, panel B), and no ALOX5 products strongly elevated assayed SASP mRNAs (FIG. 8, panel C). Prostaglandins also elevated levels of PTGS2 (COX-2) mRNA, while PGE2 elevated PTGES (FIG. 2, panel E), suggesting a positive feedback mechanism sustains prostaglandin synthase expression. In agreement with this, inhibition of COX-2 activity by CAY-10404 lowered its mRNA levels in senescent cells to non-senescent levels (FIG. 8, panel D). Notably, PGJ2 (FIG. 2, panel F) and 15d-PGJ2 (FIG. 2, panel E) strongly induced most SASP factors, except VEGF, which we had observed to be COX-2-independent (FIG. 2, panel A), and PDGF. Many prostaglandins lowered levels of several other SASP factors, though since dihomo-15d-PGJ2 was most abundant in senescent cells, it is likely that PGJ2, 15d-PGJ2, and related compounds are most important for promoting the SASP.

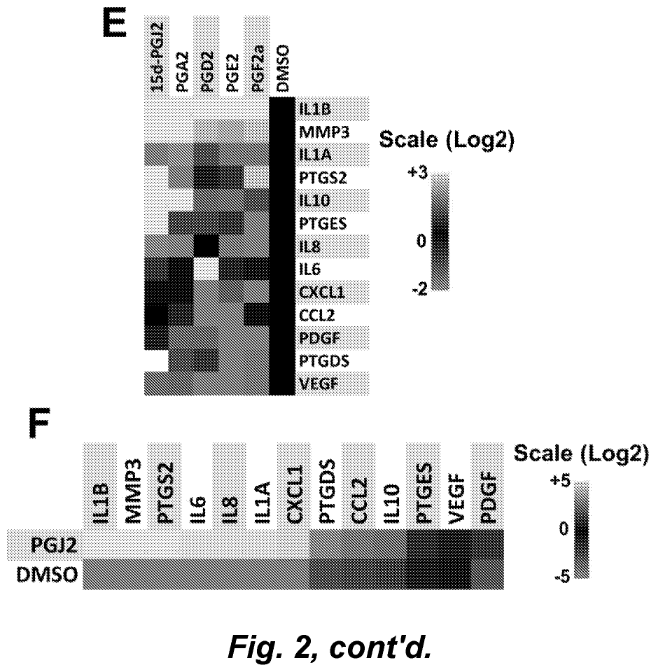

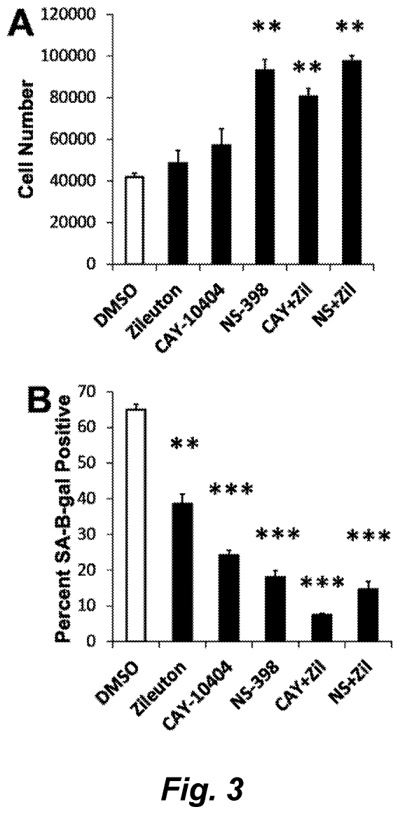

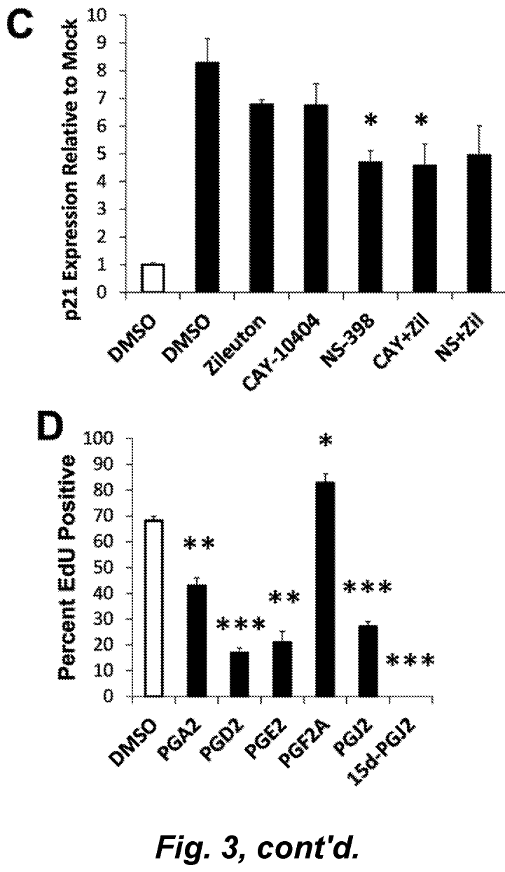

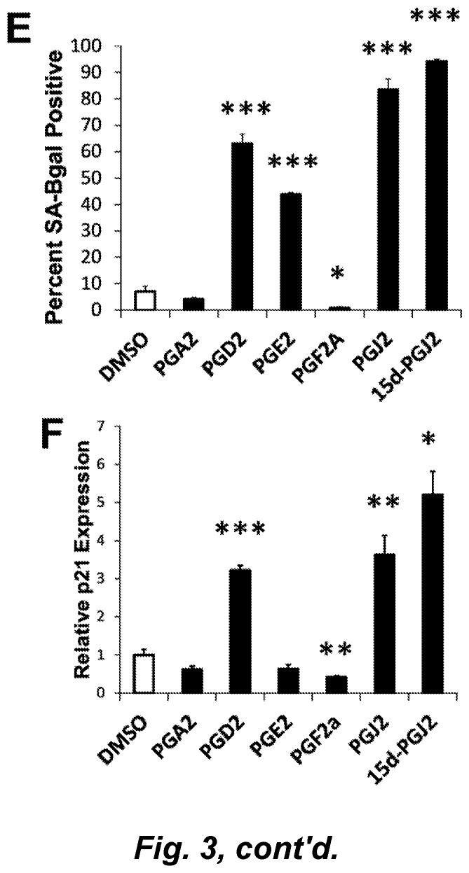

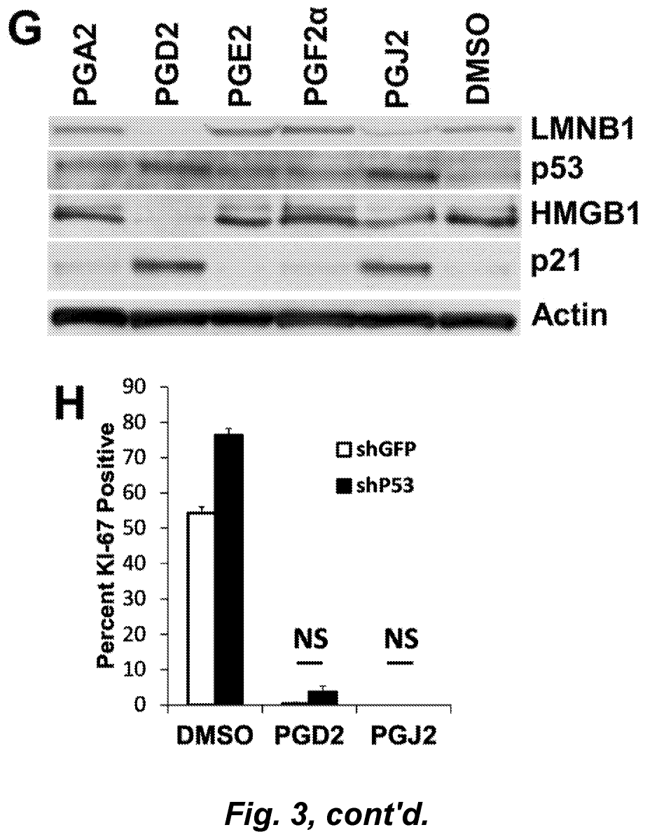

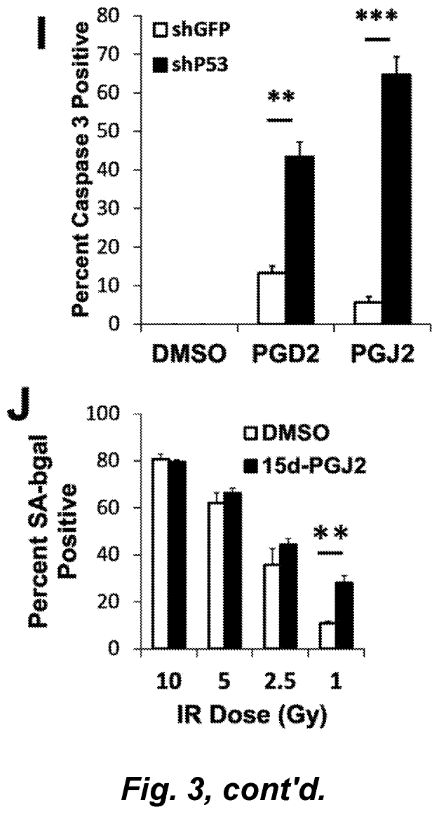

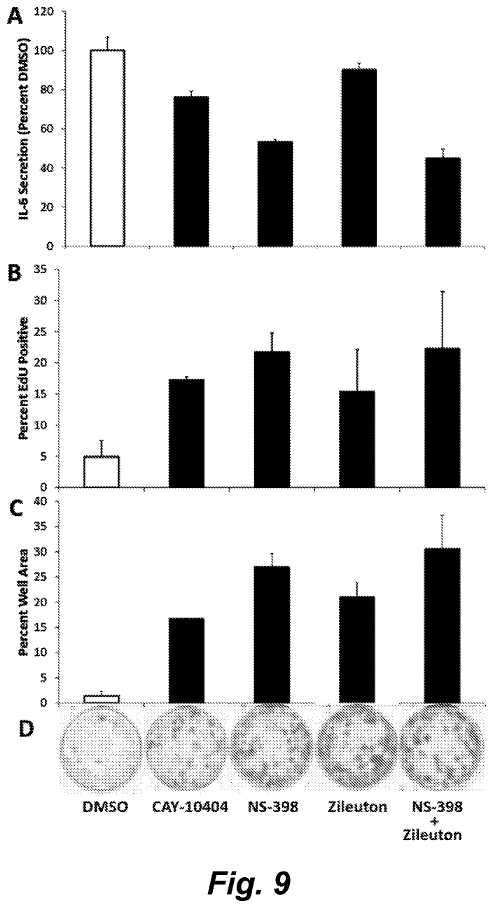

We noticed when counting cells for normalization of our IL-6 ELISA (FIG. 2, panel B) that SEN(IR) cells cultured in the presence of COX-2 inhibitors (CAY-10404 and NS-398) consistently showed an increase in cell number relative to DMSO-treated SEN(IR) cells. We therefore hypothesized that eicosanoid biosynthesis might act to reinforce mitotic arrest during senescence. To address this possibility, we treated irradiated (5 Gy) IMR-90 fibroblasts with CAY-10404, NS-398, zileuton, or combinations of each. BW-B70C retarded growth of non-senescent cells, and was therefore excluded from our analysis. While zileuton mildly increased the number of cells following irradiation, both CAY-10404 and, to a greater degree NS-398, significantly increased cell numbers, and the combination of both zileuton and CAY-10404 resulted in more cells than either inhibitor on its own (FIG. 3, panel A). These increases in cell number also correlated with decreased senescence-associated beta-galactosidase positivity (FIG. 3, panel B) as well as p21^{WAF1} (CDKN1A) mRNA levels (FIG. 3, panel C). Similar results were observed for oncogenic ras-induced senescence (Serrano et al. ('997) Cell, 88: 593-602; Catalano et al. (2005) EMBO J. 24: 170-179) in terms of IL-6 secretion (FIG. 9, panel A), EdU incorporation (Extended Data FIG. 9, panel B), and colony formation (FIG. 9, panels C-D). Thus, activation of eicosanoid biosynthesis promotes senescence.

To identify which eicosanoids are likely to promote senescence, we treated non-senescent cells with prostaglandins (PGA2, PGD2, PGE2, PGF2α, PGJ2, or 15d-PGJ2, as above) or 5-LOX pathway products (5-HETE, LTB4, LTC4, LTD4, or LTE4). Despite zileuton's activity in blunting IR- and ras-induced senescence (FIG. 3, panels A-C, FIG. 9, panels B-C), no ALOX5 products induced senescence on their own. Since mRNA levels of leukotriene receptors (CYSLTR2 and LTB4R2) declined with senescence (FIG. 8, panel B), the effects of senescence-associated leukotrienes are likely cell non-autonomous. Since ALOX5 is known to produce senescence-inducing reactive oxygen species (ROS) (Catalano et al. (2005) EMBO J. 24: 170-179), it may be the case that this activity of ALOX5, rather than leukotriene synthesis per se, reinforces mitotic arrest during senescence.

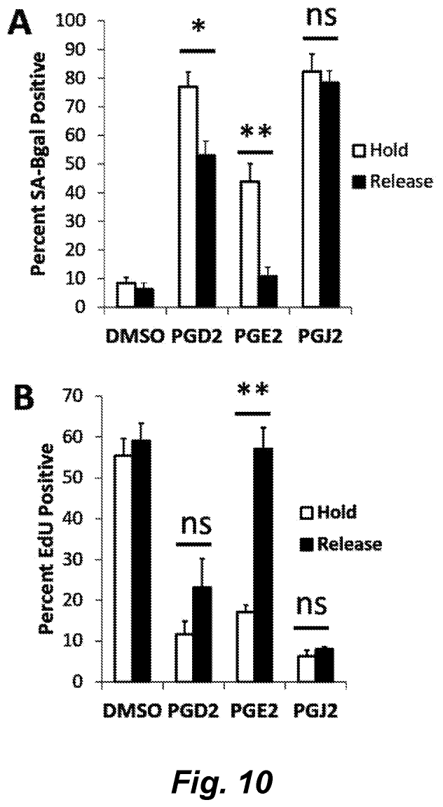

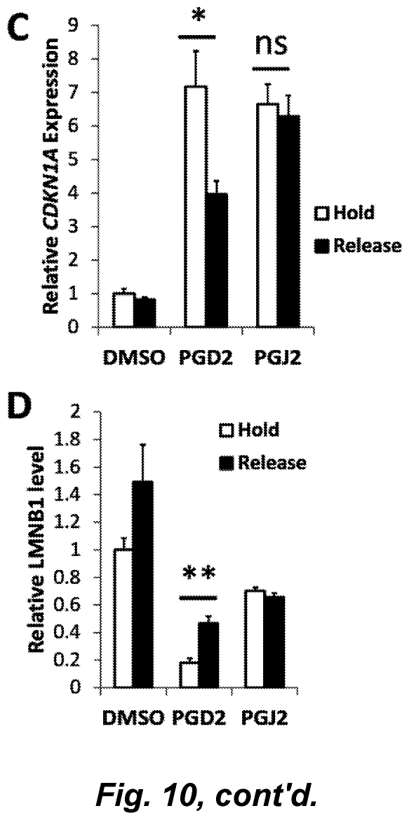

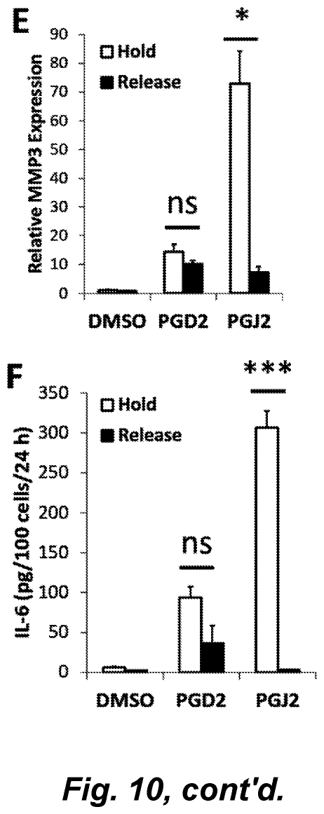

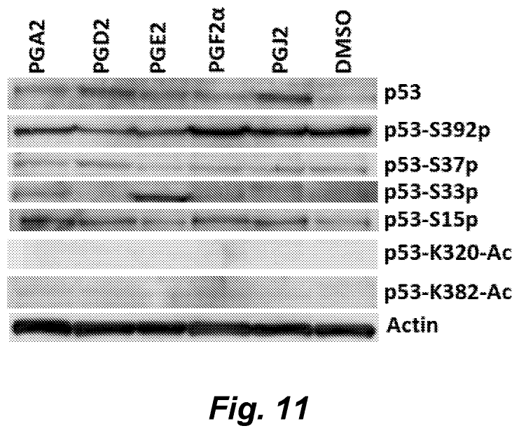

On the other hand, all prostaglandins except PGF2a slowed or arrested cell division, as measured by EdU labeling indices (FIG. 3, panel D). Indeed, PGE2, PGD2, PGJ2, and 15d-PGJ2 all induced senescence-associated beta-galactosidase (FIG. 3, panel E). However, the senescence-like phenotypes induced by PGE2 were unlikely to be senescence, as cells treated with PGE2, but not PGD2 or PGJ2, became smaller and resumed proliferation once PGE2 was removed (FIG. 10, panels A-B). Further, only prostaglandin D2 (PGD2) and its derivatives (PGJ2 and 15d-PGJ2) induced p21 gene expression (FIG. 3, panel F, FIG. 10, panel C) and protein accumulation (FIG. 3, panel G). Treatment with either PGD2 or PGJ2 stabilized p53 (FIG. 3, panel G) without increasing p53 phosphorylation or acetylation (FIG. 11). PGD2 or PGJ2, but not PGE2, treatment also resulted in reduced levels of LMNB1 and cellular HMGB1 (FIG. 3, panel G, FIG. 10, panel D), two additional biomarkers of senescence. In agreement with COX-2 being required for the SASP, we found that removal of PGD2 or PGJ2 reduced expression of MMP3 and secretion of IL-6 (FIG. 10, panels E-F). Thus, derivatives of PGD2 specifically promoted senescence.

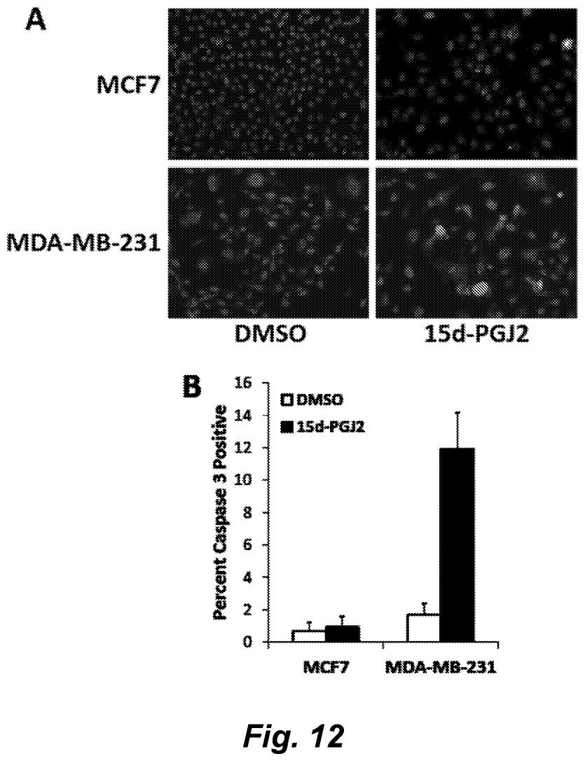

Since p53 was stabilized in response to PGD2 or PGJ2, and p21 is a transcriptional target of p53, we sought to determine if induction of senescence by PGD2 and PGJ2 was p53-dependent. We therefore depleted p53 by shRNA, followed by treatment with either PGD2 or PGJ2. While loss of p53 did not allow PGD2- or PGJ2-treated cells to continue cell division, as measured by Ki67 positivity (FIG. 3, panel H), treated cells also did not senesce, but rather underwent apoptosis, as determined by positivity for cleaved caspase 3 (FIG. 3, panel I). Since p53-depleted fibroblasts similarly undergo apoptosis following IR (Lips & Kaina (2001) Carcinogenesis, 22: 579-585), these prostaglandins appear to function in a manner similar to radiomimetics. Indeed, treatment of a p53-mutant breast cancer cell line (MDA-MB-231) with 10 μM 15d-PGJ2 induced apoptosis, while treatment of a p53-positive cell line (MCF7) did not (Extended Data FIG. 12, panels A-B), suggesting that the tumor suppressive effects of 15d-PGJ2 function in a manner similar to radiation.

Since our prostaglandin treatments were supraphysiological, we treated cells with a senescence-equivalent dose (1.4 μM) of 15d-PGJ2. This dose did not induce senescence on its own. Since inhibition of COX-2 lowered, but did not completely prevent senescence (FIG. 3, panels A-C), we considered the possibility that 15d-PGJ2 might sensitize cells to senescence-inducing stimuli, thereby reinforcing senescence. To test this possibility, we irradiated DMSO- or 15d-PGJ2-treated cells with decreasing doses of IR. At 10 Gy (our standard senescence-inducing dose), no differences were observed in terms of senescence-associated beta-galactosidase (FIG. 3, panel J), EdU incorporation (FIG. 3, panel K), and cell number (FIG. 3, panel L). However, at lower doses of IR, cells treated with 15d-PGJ2 became more senescent relative to control cells (FIG. 3, panels J-L). Thus, physiological levels of 15d-PGJ2 reinforce senescence by sensitizing cells to senescence-inducing stimuli.

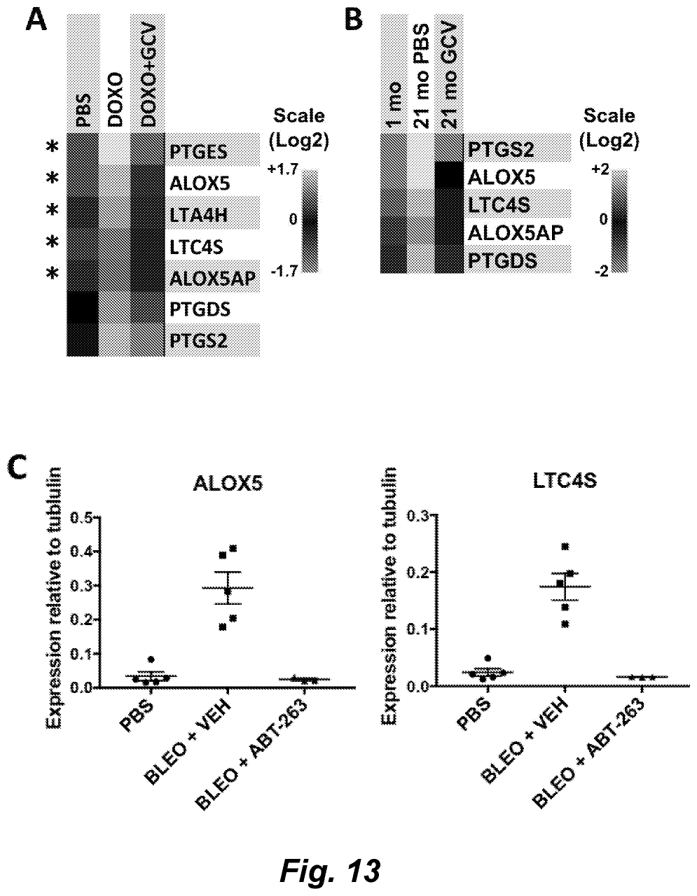

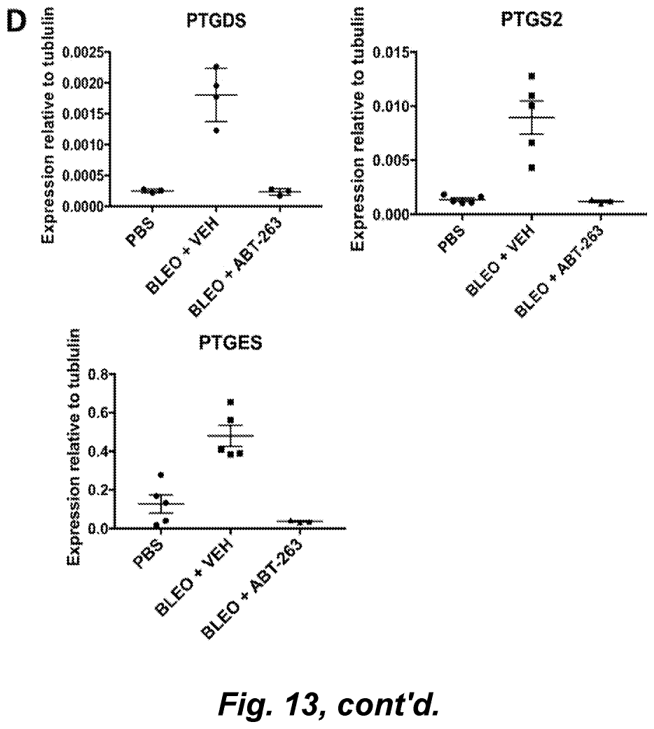

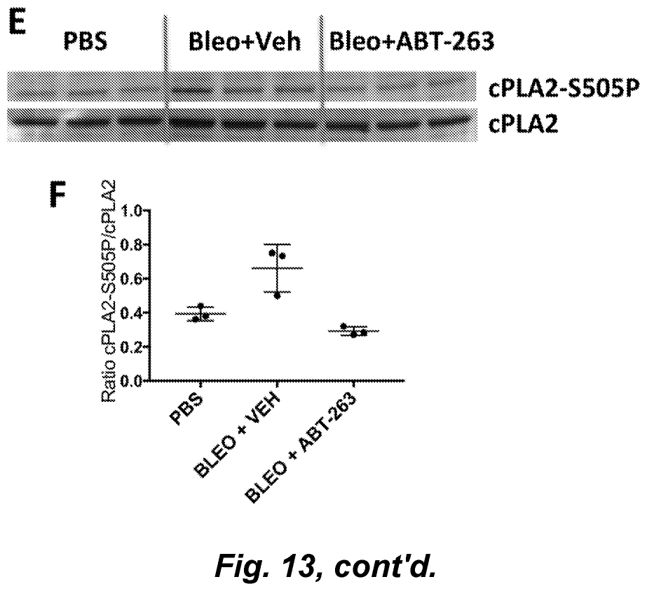

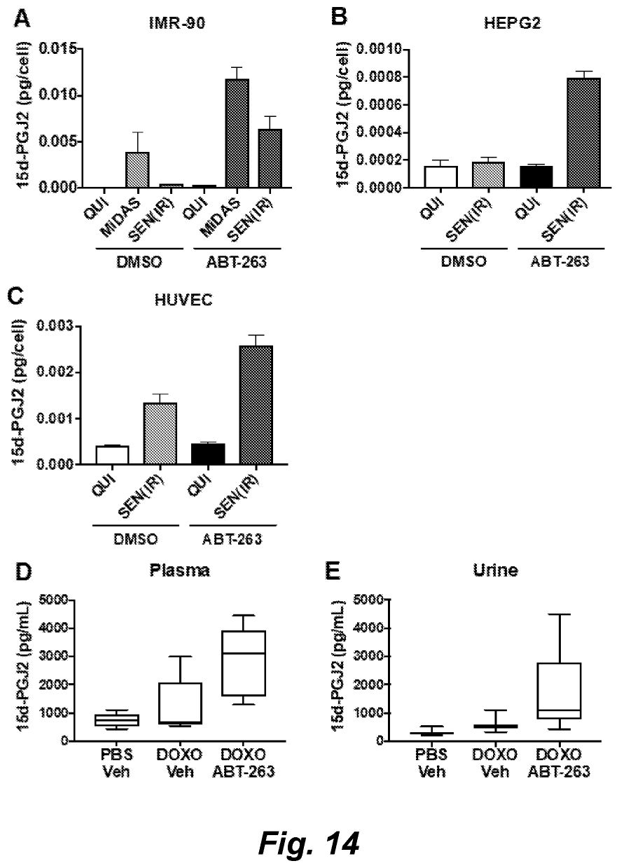

To address the biological significance of our data obtained in cultured cells, we utilized modes of known senescence induction to determine whether senescence-associated eicosanoid biosynthesis occurs in vivo. We treated p16-3MR mice with the chemotherapeutic agent doxorubicin (DOXO, FIG. 13, panel A) or aged mice for 21 months (FIG. 13, panel B). RNA expression of many leukotriene and prostaglandin synthases was significantly elevated (p<0.05). Furthermore, when mice were treated with ganciclovir (GCV) to eliminate p16-positive cells, eicosanoid synthase expression was reduced in doxorubicin-treated mice (FIG. 13, panel A). Aged mice showed a similar trend, but results were not statistically significant (FIG. 13, panel B).

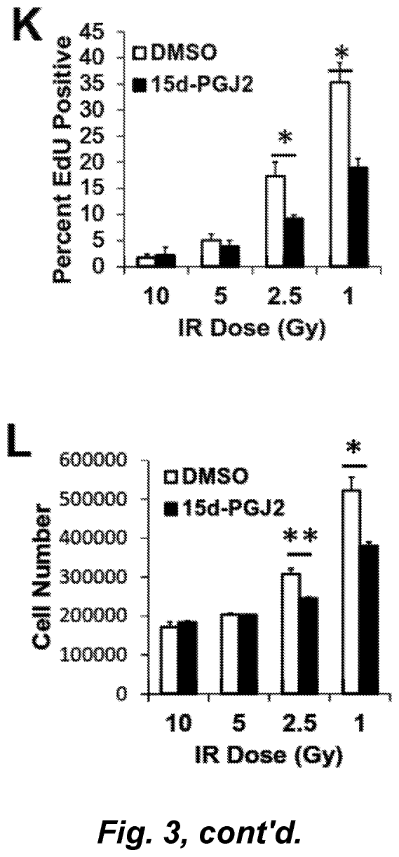

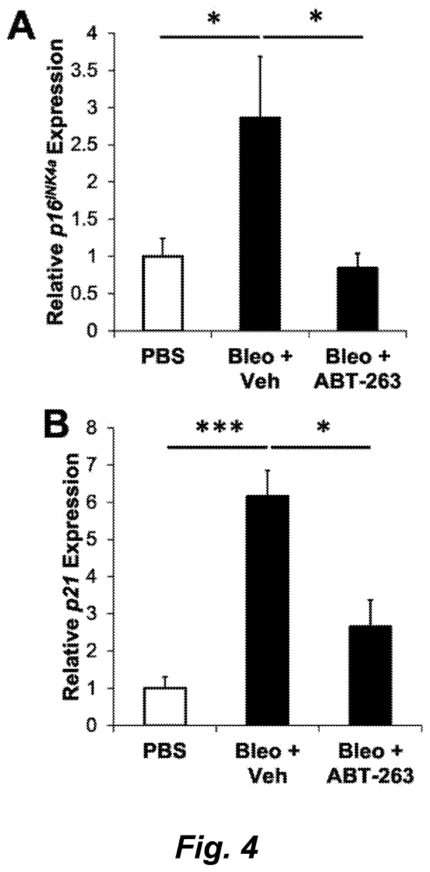

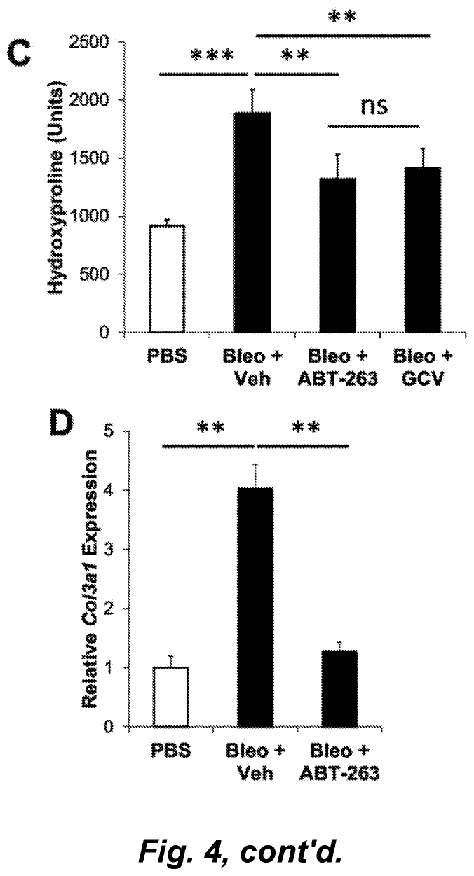

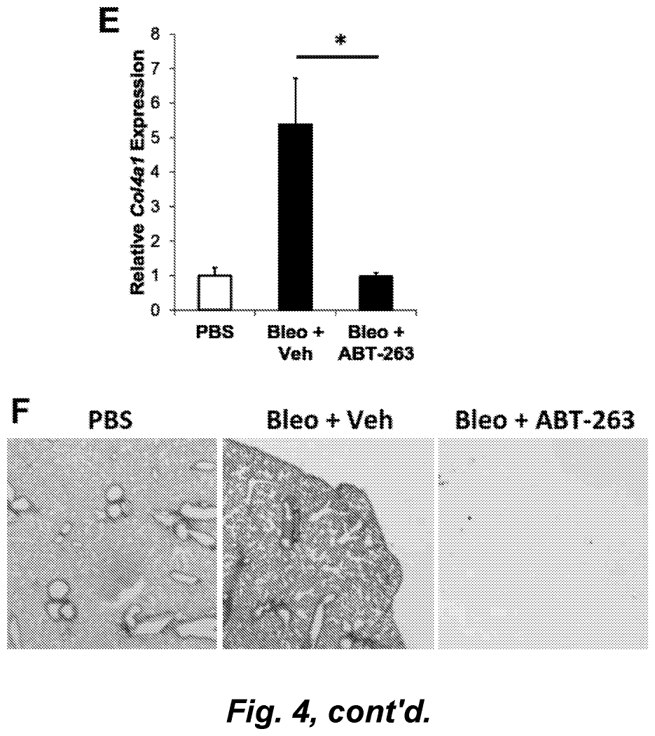

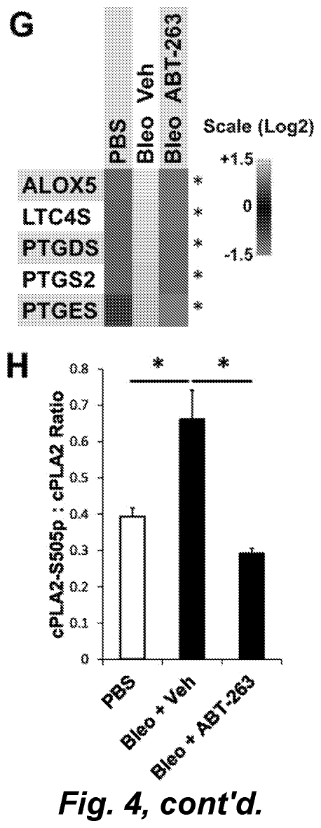

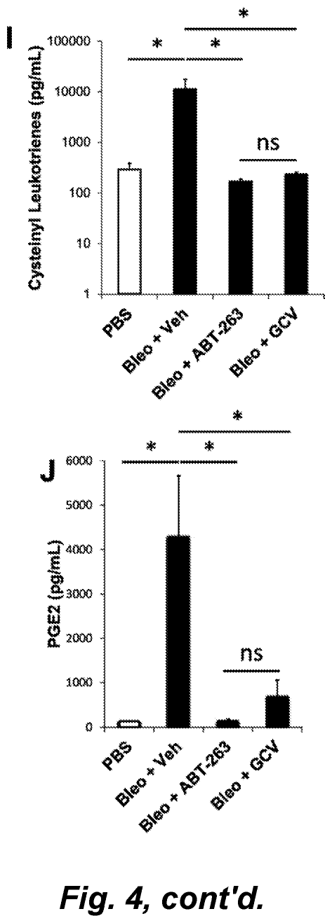

Since our initial results were observed in senescent lung fibroblasts, we sought to determine whether senescent cells were responsible for eicosanoid-driven disorders of the lung. Since eicosanoids have been previously shown to significantly contribute to fibrotic responses in the lung (Peters-Golden et al. (2002) Am. J. Respir. Crit. Care Med. 165: 229-235; Beller et al. Proc. Natl. Acad. Sci. USA, 101: 3047-3052; Oga et al. (2009) Nat. Med. 15: 1426-1430; Dackor et al. (2011) Am. J. Physiol. Lung Cell Mol. Physiol. 301: L645-655), we injured C57BL6/J and 3MR mice with bleomycin to promote fibrosis. In addition, subsets of mice were treated with ABT-263, a BCL-XL inhibitor that selectively eliminates senescent cells (Chang et al. (2016) Nat. Med. 22: 78-83; Yosef et al. (2016) Nat. Commun. 7: 11190). We also repeated key findings using the p16-3MR mouse, which allowed us to more specifically eliminate p16-positive senescent cells in bleomycin-treated mice using ganciclovir (GCV) (Demaria et al. (2014) Dev. Cell, 31: 722-733). RNA levels of p16^{INK4a} and p21^{WAF1}, two major markers of senescence, were increased 14 days after bleomycin injury and were significantly attenuated after ABT-263 and GCV treatment (FIG. 4, panels A-B). Consequently, 21 days after injury, reduced collagen content (hydroxyproline, FIG. 4, panel C) and expression (Col3a1 and Col4a1, FIG. 4, panels D and E) were measured in bleomycin-injured mice treated with ABT-263 or GCV, and histological staining of lungs with picrosirius red indicated attenuation of the fibrotic response (FIG. 4, panel F) by ABT-263. In addition, mRNA levels of Alox5, Ltc4s, Ptgs2, Ptgds, and Ptges were increased in lungs of bleomycin-injured mice, and elimination of senescent cells with ABT-263 lowered these levels to those of PBS-treated animals (FIG. 4, panel G, FIG. 13, panel A). Similarly, phosphorylation of cPLA2 was elevated in bleomycin-treated mice and lowered after ABT-263 (FIG. 4, panel H, FIG. 13, panels B-C). Importantly, levels of both cysteinyl leukotrienes and prostaglandin E2 were elevated in the bronchioalveolar lavage fluid (BALF) from bleomycin-treated animals, and were reduced after ABT-263 and GCV treatment (FIG. 4, panels I and J). These data indicate that the removal of senescent cells attenuates both eicosanoid biosynthesis and collagen deposition, and therefore is as an underlying driver of bleomycin-induced pulmonary fibrosis.

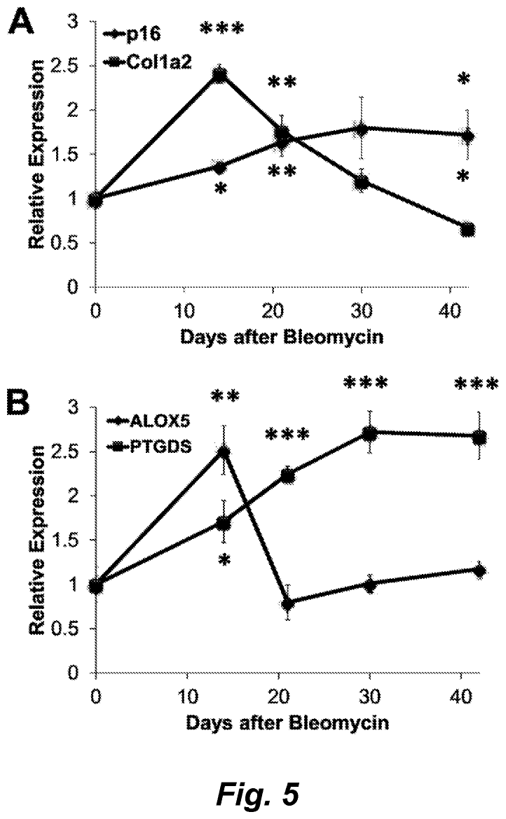

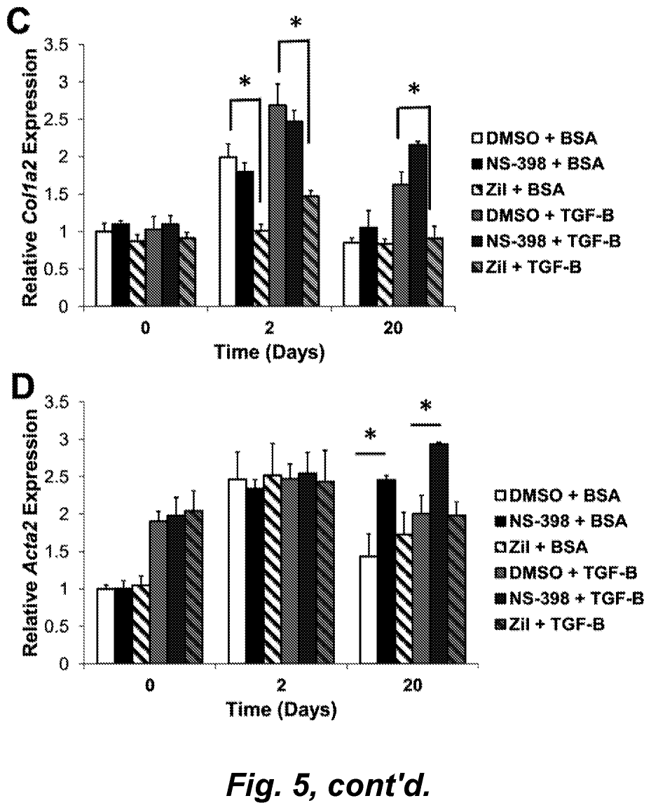

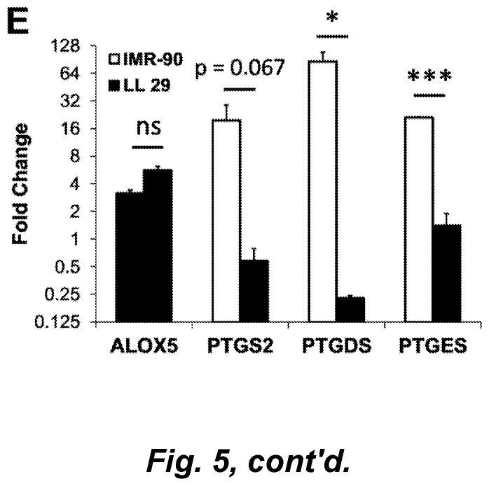

Bleomycin-induced pulmonary fibrosis resolves over time in mice (Moeller et al. (2008) Int. J. Biochem. Cell Biol. 40: 362-382; Izbicki et al. (2002) Int. J. Exp. Pathol. 83: 111-119), suggesting that either senescent cells are eventually cleared, or that the fibrosis-promoting properties of senescent cells change over time. We therefore analyzed p16 (a biomarker of senescence) or collagen (Col1A2) expression at 0, 14, 21, 30, and 42 days after bleomycin administration. We found that p16 expression was elevated at Day 14, peaked by Day 30, and remained elevated through Day 42 (FIG. 5, panel A, blue line). Despite this increase, collagen gene expression peaked at Day 14 and progressively declined over time (FIG. 5, panel A, red line). These data indicate that the fibrosis-inducing properties of senescent cells change over time. Since we observed an early elevation in leukotriene synthase expression (FIG. 1, panel F), followed by a late elevation in prostaglandin synthase expression (FIG. 1, panel E) in cultured cells, we considered the possibility that this shift in the activity of senescent cells might underlie the recovery observed in bleomycin-treated mice. Indeed, we observed an early spike in Alox5 expression following bleomycin administration (FIG. 5, panel B, blue line), followed by a more progressive elevation of Ptgds, which synthesizes PGD2. Introduction of prostaglandin D2 synthase—or administration of PGD2 or 15d-PGJ2—attenuate bleomycin-induced fibrosis (Kida et al. (2016) PLoS One, 11: e0167729; Genovese et al. (2005) Eur. Respir. J. 25: 225-234), so elevation of Ptgds is consistent with recovery from a profibrotic state.

To determine if these shifts in eicosanoid synthesis alter the fibrotic response, we generated conditioned media (CM) from control (Day 0) or SEN(IR) cells at either Day 2 or Day 20 following IR. Cells were treated with either NS-398, Zileuton (Zil) or vehicle (DMSO) for 24h prior to generation of CM. Naïve nonsenescent IMR-90 fibroblasts were then treated with CM+TGF-beta or CM+BSA, and RNA was analyzed by qPCR 24 h later. CM from senescent cells at Day 2 induced collagen expression regardless of the presence of TGF-beta (FIG. 5, panel C), and elimination of leukotriene synthesis with zileuton prevented this effect. CM from Day 20 induced collagen expression only in the presence of TGF-beta, and this was again prevented by zileuton. Conversely, zileuton had no effect on smooth muscle actin expression (ACTA2, a marker of myofibroblast differentiation), regardless of time point (FIG. 5, panel D). Induction of ACTA2 declined at Day 20 relative to Day 2, while inhibition of prostaglandin synthesis with NS-398 abrogated this decline (FIG. 5, panel D), consistent with previous findings that prostaglandins antagonize myofibroblast formation (Garrison et al. (2013) Am. J. Respir. Cell Mol. Biol. 48: 550-558; Penke et al. (2014) J. Biol. Chem. 289: 17151-17162). Together, these data support a model in which early leukotriene synthesis stimulates collagen synthesis in myofibroblasts, whereas late prostaglandin synthesis antagonizes myofibroblast differentiation and collagen expression.

Previous reports indicate that fibroblasts from patients with idiopathic pulmonary fibrosis (IPF) fail to elevate prostaglandin synthesis (Bauman et al. (2010) J. Clin. Invest. 120: 1950-1960)³⁶, or fail to respond to prostaglandin treatment (Liu et al. (2005) J. Pharmacol. Exp. Ther. 315: 678-687). We therefore used a fibroblast strain derived from a patient with IPF (LL-29) to determine if eicosanoid synthesis was perturbed in these cells. We compared RNA expression levels of ALOX5, PTGS2, PTGDS, and PTGES in control vs SEN(IR) LL-29 and IMR-90 fibroblasts (FIG. 5, panel E). In both cell strains, ALOX5 was elevated (FIG. 5, panel E), whereas senescent LL-29 fibroblasts failed to elevate any of the prostaglandin synthases (PTGS2, PTGDS, and PTGES). These data suggest that patients with IPF respond differently to senescence-inducing stimuli with regard to prostaglandin synthesis.

In conclusion, we used a combination of metabolic profiling, gene expression, and functional assays to identify eicosanoid biosynthesis as a characteristic of senescent cells. Prostaglandins, especially PGD2-derived metabolites, promoted parts of the protein SASP and acted to reinforce senescence. By comparison, while leukotrienes had little effect on senescent cells, senescence-associated leukotriene synthesis promoted pulmonary fibrosis in a mouse model. Together, our data not only identify a new activity for senescent cells, but also important phenotypic consequences for that activity, such as fibrosis.

### Supplemental Materials.

Materials and Methods

Cell Culture.

Human fetal lung fibroblasts (IMR-90) were cultured in Dulbecco's modified eagle medium (DMEM) supplemented with 10% FBS and penicillin/streptomycin. LL 29 (AnHa) cells were obtained from the ATCC and growth to confluence using the ATCC protocol, but were subcultured as described for IMR-90 before initiation of all experiments. Quiescence was induced by replacing culture media with media containing 0.2% FBS. MCF-7 and MDA-MB-231 breast cancer cells were cultured in RPMI supplemented with 10% FBS and penicillin/streptomycin. All cells were cultured at 3% O₂, and used between 25 and 40 population doublings. All cells were mycoplasma free.

Gene Expression.

RNA was extracted from cells or tissues using commercially available kits (Isolate II—Bioline for cells; Direct-zol—Zymo for tissues) according to the manufacturer's instructions. cDNA synthesis was performed using a High Capacity cDNA Reverse Transcription Kit (Thermo Fisher) according to the manufacturer's instructions. Quantitative PCR was performed on a LightCycler 480 II (Roche) using primers and probes designed for the Universal Probe Library. Primers and probes used for human transcripts were ACTB: 5′-CCAACCGCGAGAAGATGA (SEQ ID NO:1), 5′-TCCATCACGATGCCAGTG (SEQ ID NO:2), Probe #64; TUBA: 5′-cttcgtctccgccatcag (SEQ ID NO:3), 5′-ttgccaatctggacacca (SEQ ID NO:4), Probe #58; ALOX5: 5′-ccacggagatggtagagtgc (SEQ ID NO:5), 5′-cgatgaaaatgttcccttgc (SEQ ID NO:6), Probe #64; ALOX5AP: 5′-ggtctgcggggctacttt (SEQ ID NO:7), 5′-tgcctcacaaacaagtacatcag (SEQ ID NO:8), Probe #18; ALOX12: 5′-tctcagatggaggaatttttgat (SEQ ID NO:9), 5′-agccaggtcgtcaggag (SEQ ID NO:10), Probe #32; ALOX15: 5′-aatcgtgagtctccactataagacag (SEQ ID NO:11), 5′-gcctgtaaagagacaggaaacc (SEQ ID NO:12), Probe #76; ALOX15B: 5′-gatcttcaacttccggaggac (SEQ ID NO:13), 5′-actgggaggcgaagaagg (SEQ ID NO:14), Probe #51; AKR1C1: 5′-catgcctgtcctgggattt (SEQ ID NO:15), 5′-agaatcaatatggcggaagc (SEQ ID NO:16), Probe #49; AKR1C2: 5′-ctatgcgcctgcagaggt (SEQ ID NO:17), 5′-acctgctcctcattattgtaaacat (SEQ ID NO:18), Probe #31; AKR1C3: 5′-cattggggtgtcaaacttca (SEQ ID NO:19), 5′-ccggttgaaatacggatgac (SEQ ID NO:20), Probe #78; CYSLTR2: 5′-gacgggtgatttctgcattt (SEQ ID NO:21), 5′-ctctcttgaagttttcaattgtgc (SEQ ID NO:22), Probe #18; DPEP1: 5′-gacgtcctgaggctggtg (SEQ ID NO:23), 5′-ggtgcaggaaatgtaattgttg (SEQ ID NO:24), Probe #9 (SEQ ID NO:25), ELOVL2: 5′-cacacttctctccgcgtacat (SEQ ID NO:26), 5′-gttgtagcctccttcccaagt (SEQ ID NO:27), Probe #53; ELOVL4: 5′-catttggcccatggattc (SEQ ID NO:28), 5′-ccaatggtcacatggaattg (SEQ ID NO:29), Probe #31; ELOVL5: 5′-cccttccatgcgtccata (SEQ ID NO:30), 5′-gattgtcagcacaaactgaagc (SEQ ID NO:31), Probe #31; EPHX2: 5′-ttctgctggacaccctgaa (SEQ ID NO:32), 5′-ttcagattagccccgatgtc (SEQ ID NO:33), Probe #45; GPX4: 5′-ttcccgtgtaaccagttcg (SEQ ID NO:34), 5′-cggcgaactctttgatctct (SEQ ID NO:35), Probe #36; LTA4H: 5′-ctgctctcacggtccagtc (SEQ ID NO:36), 5′-ttttctattgtaaggtcctttgtatcc (SEQ ID NO:37), Probe #64; LTBR2: 5′-tgctgcttaacctttcagctt (SEQ ID NO:38), 5′-atccttctgggcctacaggt (SEQ ID NO:39), Probe #33; LTC4S: 5′-accatgaaggacgaggtagc (SEQ ID NO:40), 5′-tgcagggagaagtaggcttg (SEQ ID NO:41), Probe #18; PTGDS: 5′-ccaacttccagcaggacaag (SEQ ID NO:42), 5′-ccacagacttgcacatggac (SEQ ID NO:43), Probe #36; PTGES: 5′-agagatgcctgcccacag (SEQ ID NO:44), 5′-tggccaccacgtacatctt (SEQ ID NO:45), Probe #4; PTGES2: 5′-gcagggctgagatcaagttc (SEQ ID NO:46), 5′-gacagaggagtcatttagttgttgc (SEQ ID NO:47), Probe #49; PTGES3 (SEQ ID NO:48), 5′-ttaacaaaagaaagggcaaagc (SEQ ID NO:49), 5′-tcagagaaacgatcaaaattagacat (SEQ ID NO:50), Probe #48; PTGIS: 5′-gatttttgatgtgcagcttcc (SEQ ID NO:51), 5′-gtgagtgcctggagctctct (SEQ ID NO:52), Probe #45; PTGR1: 5′-atgatggggcagcaagtg (SEQ ID NO:53), 5′-cccatcagaaatggagtgc (SEQ ID NO:54), Probe #64; PTGS1: 5′-tccatgttggtggactatgg (SEQ ID NO:55), 5′-gtggtggtccatgttcctg (SEQ ID NO:56), Probe #81; PTGS2: 5′-gatccccagggctcaaac (SEQ ID NO:57), 5′-tcaccgtaaatatgatttaagtccac (SEQ ID NO:58), Probe #61; TBXAS1: 5′-ctgccctatctggacatggt (SEQ ID NO:59), 5′-tgcctcccgtgtgaatct (SEQ ID NO:60), Probe #73; AREG: 5′-tgatcctcacagctgttgct (SEQ ID NO:61), 5′-tccattctcttgtcgaagtttct (SEQ ID NO:62), Probe #73; CCL2: 5′-AGTCTCTGCCGCCCTTCT (SEQ ID NO:63), 5′-GTGACTGGGGCATTGATTG (SEQ ID NO:64), Probe #40; CCL27: 5′-taggctgagcaacatgaagg (SEQ ID NO:65), 5′-gctgggtggcagtaggaat (SEQ ID NO:66), Probe #18; CXCL1: 5′-gctgaacagtgacaaatccaac (SEQ ID NO:67), 5′-cttcaggaacagccaccagt (SEQ ID NO:68), Probe #52; HGF: 5′-gattggatcaggaccatgtga (SEQ ID NO:69), 5′-ccattctcattttatgttgctca (SEQ ID NO:70), Probe #49; ILIA: 5′-GGTTGAGTTTAAGCCAATCCA (SEQ ID NO:71), 5′-TGCTGACCTAGGCTTGATGA (SEQ ID NO:72), Probe #6; IL1B: 5′-ctgtcctgcgtgttgaaaga (SEQ ID NO:73), 5′-ttgggtaatttttgggatctaca (SEQ ID NO:74), Probe #78; IL6: 5′-GCCCAGCTATGAACTCCTTCT (SEQ ID NO:75), 5′-gaaggcagcaggcaacac (SEQ ID NO:76), Probe #45; IL8: 5′-agacagcagagcacacaagc (SEQ ID NO:77), 5′-atggttccttccggtggt (SEQ ID NO:78), Probe #72; IL10: 5′-gatgccttcagcagagtgaa (SEQ ID NO:79), 5′-gcaacccaggtaacccttaaa (SEQ ID NO:80), Probe #67; MMP3: 5′-caaaacatatttctttgtagaggacaa (SEQ ID NO:81), 5′-ttcagctatttgcttgggaaa (SEQ ID NO:82), Probe #36; PDGFA: 5′-gcagtcagatccacagcatc (SEQ ID NO:83), 5′-tccaaagaatcctcactcccta (SEQ ID NO:84), Probe #80; VEGF: 5′-ggattttggaaaccagcaga (SEQ ID NO:85), 5′-ccgtctctctcttcctcgac (SEQ ID NO:86), Probe #50; P21WAF1(CDKN1A): 5′-tcactgtcttgtacccttgtgc (SEQ ID NO:87), 5′-ggcgtttggagtggtagaaa (SEQ ID NO:88), Probe #32; and LMNB1: 5′-ttggatgctcttggggttc (SEQ ID NO:89), 5′-aagcagctggagtggttgtt (SEQ ID NO:90), Probe #31. Murine primers and probes were Actb: 5′-CTAAGGCCAACCGTGAAAAG (SEQ ID NO:91), 5′-ACCAGAGGCATACAGGGACA (SEQ ID NO:92), Probe #64; Tuba: 5′-ctggaacccacggtcatc (SEQ ID NO:93), 5′-gtggccacgagcatagttatt (SEQ ID NO:94), Probe #88; Alox5: 5′-aggcacggcaaaaacagtat (SEQ ID NO:95), 5′-tgtggcatttggcatcaata (SEQ ID NO:96), Probe #58; Ltc4s: 5′-ctcttctggctaccgtcacc (SEQ ID NO:97), 5′-aagcccttcgtgcagagat (SEQ ID NO:98), Probe #7; Ptgds: 5′-ggctcctggacactacaccta (SEQ ID NO:99), 5′-atagttggcctccaccactg (SEQ ID NO:100), Probe #89; Ptges: 5′-agcacactgctggtcatcaa (SEQ ID NO:101), 5′-cagcctcatctggcctgt (SEQ ID NO:102), Probe #83; Ptgs2: 5′-gggagtctggaacattgtgaa (SEQ ID NO:103), 5′-gtgcacattgtaagtaggtggact (SEQ ID NO:104), Probe #4; Col3a1: 5′-ctcctggtgagcgaggac (SEQ ID NO:105), 5′-gaccaggttgcccatcact (SEQ ID NO:106), Probe #1; Col4a1: 5′-tggcacaaaagggacgag (SEQ ID NO:107), 5′-ggccaggaataccaggaag (SEQ ID NO:108), Probe #1; p21WAF1(Cdkn1a): 5′-TCCACAGCGATATCCAGACA (SEQ ID NO:109), 5′-GGACATCACCAGGATTGGAC (SEQ ID NO:110), Probe #21; and p16INK4a(Cdkn2a): 5′-AACTCTTTCGGTCGTACCCC (SEQ ID NO:111), 5′-TCCTCGCAGTTCGAATCTG (SEQ ID NO:112), Probe: Custom.

Induction of Senescence.

Senescence was induced by irradiation with 10 Gy of ionizing radiation. Non-senescent controls (proliferating or quiescent) were placed in the irradiator for an identical period of time without turning on the irradiator. Oncogene-induced senescence was induced via lentiviral overexpression of HRAS^{V12}, as described previously (Wiley et al. (2016) Cell Metab. 23: 303-314).

Generation of Conditioned Media.

Conditioned media were generated by culturing IMR-90 fibroblasts in the presence of serum-free DMEM supplemented with penicillin/streptomycin for 24 hours before harvest, follow by tyrpsinization and cell counting. Prior to generation of conditioned media, donor cells were subject to 10 Gy IR for 2 or 20 days prior to addition of serum free media. Day 0 consisted only of a sham irradiation protocol. During the final 24 hours before addition of serum free media, cells were cultured in the presence of DMSO, NS-398 (XXmg/mL), or zileuton (XX mg./mL), cells were serially washed with PBS 4× before addition of SFM. 200,000 cell equivalents/mL were used for induction studies supplemented with XX×g/mL TGF-beta or carrier (BSA). The CM+/−TGF-beta were applied to serum-starved naïve non-senescent IMR-90 fibroblasts for 24 hours, and the resulting cells were analyzed by qPCR.

Senescence-Associated Beta-Galactosidase.

SA-Bgal activity was detected as described previously (Dimri et al. (1995) Proc. Natl. Acad. Sci. USA, 92: 9363-9367) using a commercial kit (Biovision).

Immunofluorescence.

For EdU labeling, cells were cultured with EdU (10 μM) for 24 h, fixed in 4% buffered formalin for 10 min, washed in PBS, and permeabilized in 0.5% Triton X-100 for 30 min. Cells were treated as instructed by the manufacturer Life Technologies Cat #C10337). For immunofluorescence, cells were blocked in 10% normal goat serum for 30 min, washed, and incubated overnight in antibodies to Ki67 (Abcam) or cleaved caspase 3 (Cell Signaling). Cell were then washed, incubated in fluorescent secondary antibodies, and mounted using vectashield mounting medium with DAPI.

Immunoblots.

Cells were lysed in 5% SDS in 10 mM Tris, pH 7.4, and protein content determined by BCA assay. 20 μg protein was separated by electrophoresis and transferred to PVDF membranes. Membranes were blocked in TBST+5% BSA, incubated overnight with primary antibody, washed in TBST, incubated with HRP-conjugated secondary antibody for 30 min, and visualized by chemiluminescence. Antibodies to cPLA2 (phospho-5505 and unmodified), phosphorylated moieties of p53, and total p38MAPK were from Cell Signaling. LMNB1 and HMGB1 were from Abcam. Phospho-p38 was from PhosphoSolutions. p21(CDKN1A) was from Novus.

IL-6 ELISA.

3×10⁴ cells in 12-well plates were treated as indicated in the text, and cultured in 0.5-1 ml serum-free DMEM for 24 hr. CM were collected and clarified at 2,000×g for 10 min. Supernatants were transferred to a tube; cells were trypsinized and counted. CM (2.5 μl) were analyzed by bead-based ELISAs (AlphaLISA, Perkin-Elmer) as instructed by the manufacturer and normalized to cell number.

Cysteinyl Leukotriene ELISA.

BALF were extracted and analyzed by ELISAs (Cysteinyl Leukotriene, Cayman Chemical) and (Amersham Leukotriene C4/D4/E4 Biotrak, GE Healthcare) according to the manufacturer's instructions. Leukotrienes were quantified as pg/mL.

Reporter Assays.

Lentiviral NF-κB reporter constructs (SA Biosciences) and baculoviral PPAR gamma constructs (Signosis) were transduced into proliferating cells. Following treatment, luciferase was extracted using Passive Lysis Buffer (Promega) and analyzed using a commercially available kit (Promega) using a Perkin-Elmer Victor™ X3 luminometer.

Animals.

Animal experiments were conducted using a protocol approved by the Institutional Animal Care and Use Committee of the Buck Institute for Research on Aging. For Doxo treatments, 10-16 wk old p16-3MR mice received one intraperitoneal (i.p.) injection of 10 mg/kg of doxorubicin hydrochloride in PBS, and treated 5 days later with GCV or vehicle. GCV was administered via daily i.p. injections for 5 consecutive days at 25 mg/kg in PBS. Control mice were injected with an equal volume of PBS. Mice were euthanized and tissues were collected 10 days after doxorubicin challenge. For aging studies, p16-3MR mice were aged naturally for 21 mo, receiving 25 mg/kg GCV or an identical volume of PBS for 5 days each month from 6 mo of age until 21 mo, at which point mice were euthanized and tissues were collected for analysis.

To induce pulmonary fibrosis, WT C57BL/6J (WT) and p16-3MR mice were administered 2 U/kg body weight of bleomycin intratracheally. Control animals from both cohorts received PBS intratracheally. Bleomycin-treated animals showing less than 10% weight loss were excluded from the study. Injured animals were treated with ganciclovir (GCV) (25 mg/kg in PBS), ABT-263 (50 mg/kg in 10% EtOH, 20% PEG-400, and 70% Phosal 40), or a corresponding control for 7 days starting one-week post injury of Bleomycin. Animals were euthanized either 14 or 21 days after bleomycin challenge. To collect brochoalveolar lavage fluid (BALF), 1 ml of PBS was injected intratracheally and approximately 0.8 ml was retrieved. Bronchioalveolar lavage fluid was then centrifuged at 500×g for 10 minutes. Pelleted cells were suspended in Isoton solution and counted using the Beckman Z1 Coulter Counter. Lungs were collected and distributed as follows: left lung was used for hydroxyproline measurement or was paraffin-embedded, and other lobes were used for RNA/protein extraction.

Assessment of Pulmonary Fibrosis.

The extent of fibrosis indices in the lungs of bleomycin-challenged mice was assessed with established markers (Huang et al. (2015) Exp. Gerontol. 61:62-75). Collagen deposition was quantified using hydroxyproline measurement and picrosirus red staining and quantification. A) Measurement of Hydroxyproline: hydroxyproline content of the lung was measured as previously described (Id.). The left lobe of the lung was homogenized briefly in 1 ml of water and incubated in 50% TCA (trichloroacetic acid) for 20 minutes. Samples were then hydrolyzed with 6N HCl at 110° C. for 24 h. Samples were reconstituted in 2 ml of water and mixed at room temperature for 2 h. After incubation samples were mixed in equal parts chloramine T (1.4% chloramine T in 0.5 M sodium acetate and 10% isopropanol) and Ehrlich's solution (1M p-dimethylaminobenzaldehyde in 70% isopropanol and 30% perchloric acid) and heated at 65° C. for 15 minutes. The absorbance of each sample was then measured at 550 nm. Standard curves were generated for each experiment using a hydroxyproline standard (trans-4-hydro-1-proline). Results are expressed as mg hydroxyproline/mL tissue. B) Picrosirius red staining. Five-μm thick paraffin sections, layered on silane-coated slides, were stained with picrosirius red (PSR) to quantify parenchymal collagen deposition. The level of staining was assessed with ImagePro software v6.2 (Media Cybernetics, Inc. Bethesda, Md., United States) and expressed as a percentage of the total area of the image analyzed. To minimize variation, samples were processed under identical conditions. Five slides of the entire left lung per animal were quantified by an operator blinded to the treatments.

Chemicals and Standards.

Ammonium acetate was obtained from Sigma Aldrich (St. Louis, Mo.). HPLC-grade solvents acetonitrile and methanol were purchased from Fisher Scientific (Pittsburgh, Pa., USA) and VWR (Radnor, Pa., USA). Deionized water was generated in-house for mobile phase preparation. PGA2, PGD2, PGE2, PGF2α, PGJ2, 15d-PGJ2, LTB4, LTC4, LTD4, LTE4, and 5-HETE were from Cayman Chemical.

Liquid-Liquid Extraction (LLE) of Lipids.

Lipid extraction was performed based on previously reported protocols (Folch et al. (1951) J. Biol. Chem. 191: 833-841; Bligh & Dyer (1959) Can. J. Biochem. Physiol. 37: 911-917) with some modifications. IMR90 proliferating, quiescent, and senescent (IR) cells were rinsed with phosphate-buffered saline (PBS) and quenched using 1 mL 50% methanol with 2 μg/mL 13C1-leucine and 5ηg/mL hexanesulfonic acid added as internal standards. A volume of 2 mL of chloroform with 1 μg/mL heptadecanoic acid was added to each sample and mixed for 10 min at 4° C. Samples were centrifuged at 4,000 g for 15 min at 4° C., to fully separate aqueous and organic layers. After centrifugation, 700 μL of the aqueous layer and 1.5 mL of the organic layer were recovered and concentrated by speedvac and N₂, respectively. Both aqueous and organic fractions were reconstituted in 100 μL of 50% methanol and chloroform prior to liquid-chromatography mass spectrometry (LC-MS) analysis.

Solid Phase Extraction (SPE) of Lipids.

Lipid extraction was performed based on a previously reported protocol (Harkewicz et al. (2007) J. Biol. Chem. 282: 2899-2910) with some modifications. IMR-90 proliferating, quiescent, and senescent (IR) cells were rinsed with PBS and quenched using 1 mL methanol with 2 μg/mL 13C1-leucine, 1 μg/mL heptadecanoic acid, and 5ηg/mL hexanesulfonic acid added as internal standards. A volume of 1 mL of PBS was added to each sample and mixed for 10 min. Samples were centrifuged at 4,000 g for 15 min at 4° C. Following centrifugation, eicosanoids were separated using Phenomenex Strata-X polymeric sorbent columns connected to a vacuum manifold. Columns were pre-washed using 2 mL of methanol followed by 2 mL of water. Samples were loaded onto columns, washed with 2 mL of 90:10 water: methanol, and then eluted with 1 mL of 100% methanol. Extracts were concentrated by speedvac and reconstituted in 100 μL of 100% methanol prior to LC-MS analysis.

High-Pressure Liquid Chromatography Quadrupole-Time-of-Flight Mass Spectrometry (HPLC-QTOF-MS).

LC-MS analyses were performed on a Agilent 6520 QTOF mass spectrometer coupled to a Agilent 1260 Infinity liquid chromatography system consisting of the following modules: u-degasser (G1322A), binary pump (G1312B), thermostated column compartment (G1330B), and HiPALS auto sampler (G1367E). Chromatographic separation of cellular extracts was performed on a Phenomenex Luna NH2 (2.0 mm×150 mm, 3.0 μM) column. The mobile phase included A:20 mM ammonium acetate and 5% acetonitrile, pH9.5 and B:acetonitrile. The gradient is as follows: 0 to 20 min, 95-10% B, 25-30 min 10% B, and 30.1-35 min 95% B. LC conditions included auto sampler temperature 4° C., injection volume 10 μL and solvent flow-rate 0.3 mL/min. Mass spectrometric analyses were performed using the following ionization parameters: gas temperature (TEM) 350° C.; drying gas, 9 L/min; Vcap, 2500V; nebulizer, 35 psig; fragmentor, 125V; and skimmer, 65V. MS1 acquisition was operated in the negative ion scanning mode for a mass range of 50-1000 m/z.

LC-MS data was acquired and analyzed using Agilent MassHunter Workstation (B.05.00), Agilent MassHunter Qualitative Analysis Software (B.07.00), Mass Profiler Professional (B.12.0), and Microsoft Excel 2007 (Redmond, Wash., USA). To perform a comparative quantitation of metabolites, peak areas were assigned using Agilent MassHunter Qualitative Analysis Software in combination with the Find by Formula (FBF) algorithm. Peak areas were normalized by total protein or cell number.

High-Pressure Liquid Chromatography Quadropole Ion Trap Mass Spectrometry (HPLC-QTRAP-MS).

For HPLC-MS quantitation, HPLC was performed using a Shimadzu UFLC prominence system fitted with following modules: CBM-20A (Communication bus module), DGU-A₃(degasser), two LC-20AD (liquid chromatography, binary pump), SIL-20AC HT (auto sampler) and connected to a Phenomenex Luna NH₂ (2.0 mm×150 mm, 3.0 μM) column. The solvent system was A=20 mM ammonium acetate pH 9.5 with 5% acetonitrile and B=acetonitrile. The starting gradient conditions were 95% B at a flow rate of 0.3 mL/min. The following gradient program was used: 0 to 20 min, 95-10% B, 25-30 min 10% B, and 30.1-35 min 95% B. Samples were kept at +4° C., and the injection volume was 10 μL.

Mass spectrometric analysis experiments was conducted using negative ion electrospray ionization in the multiple reaction monitoring mode (MRM) on an AbSciex 4000 QTRAP (Foster City, Calif., USA) mass spectrometer fitted with a TurboV™ ion source. The ionization parameters were set as follows: curtain gas (CAD); 20 psi; collision gas: medium; ion spray voltage (IS): −4500V; Temperature (TEM): 550° C.; Ion source Gas 1 (GS1); 60 psi; and Ion source Gas 2 (GS2): 50 psi. The compound parameters were established using the appropriate standards. The compound parameters were set as follows: entrance potential (EP): −10.0V; and collision cell exit potential (CXP): −5V. ABSciex Analyst® v1.6.1 was used for all forms of data acquisition and method development.

ABSCIEX ANALYST® v1.6.1 was used for all forms of data acquisition and an in-depth analysis of the HPLC-MS data, specifically for calculating the peak areas for the identified features from cellular extracts. Peak areas were normalized by total protein.

Statistical Analysis.

All data were presented as means+/−SEM. Comparisons between groups were performed using 2-tailed student t-test, Mann-Whitney U test (for comparison of data sets with non-equivalent variances), or 2-way ANOVA, as appropriate. Heat maps used P<0.05 for all markers of significance. Significance was otherwise indicated by *=P<0.05, **=P<0.01, ***=P<0.001.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1: A method of identifying elevated levels of senescent cells in a mammal, said method comprising:
determining the levels of one or more indicators of senescent cells in a biological sample from said mammal, wherein said one or more indicators are selected from the group consisting of an eicosanoid, an eicosanoid precursor, leukotriene A4 (LTA4), leukotriene B4 (LTB4), PGD2, and 5-HETE; and
wherein an elevated level of said one or more indicators is an indicator of elevated levels of senescent cells in said mammal.

2-26. (canceled)

27: A method of treating a pathology characterized by elevated levels of senescent cells in a mammal, said method comprising:
administering an effective amount of one or more senolytic agents to a mammal determined to have elevated levels of one or more indicators of senescent cells wherein said one or more indicators are selected from the group consisting of an eicosanoid, an eicosanoid precursor, leukotriene A4 (LTA4), leukotriene B4 (LTB4), PDG2, and 5-HETE.

28: The method of claim 27, wherein said mammal is determined to have elevated levels of one or more indicators selected from the group consisting of an eicosanoid precursor, leukotriene A4 (LTA4), and leukotriene B4 (LTB4).

29: The method of claim 27, wherein administering comprises administering a therapeutically effective course of therapy of a small molecule senolytic agent wherein the senolytic agent selectively kills senescent cells in comparison with non-senescent cells.

30: The method of claim 27, wherein the senolytic agent is a specific inhibitor of MDM2, Bcl-xL or Akt.

31. (canceled)

32: The method of claim 27, wherein the senolytic agent comprises an inhibitor of MDM2.

33: The method of claim 32, wherein:
the MDM2 inhibitor comprises an imidazoline compound, a dihydroimidazothiazole compound, a spiro-oxindole compound, a benzodiazepine compound, or a piperidinone; or
the MDM2 inhibitor is selected from the group consisting of Nutlin-1, Nutlin-2, RG-7112, RG7388, R05503781, DS-3032b, MI-63, MI-126, MI-122, MI-142, MI-147, MI-18, MI-219, MI-220, MI-221, MI-773, 3-(4-chlorophenyl)-34(1-(hydroxymethyl)cyclopropyl)methoxy)-2-(4-nitrobenzyl)isoindolin-1-one, Serdemetan, AM-8553, CGM097, RO-2443, and RO-5963; or
the senolytic agent comprises an imidazoline compound.

34-35. (canceled)

36: The method of claim 33, wherein the senolytic agent comprises an imidazoline compound having the structure: 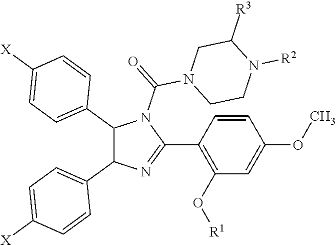
or a pharmaceutically acceptable salt thereof; wherein:
X is halide;
R¹ is alkyl, R² is —H or heteroalkyl, and
R³ is —H or ═O.

37: The method of claim 36, wherein:
the imidazoline compound is selected from the group consisting of nutlin-1, nutlin-2, and nutlin-3; or
the imidazoline compound comprises a 4-[[(4S,5R)-4,5-bis(4-chlorophenyl)-4,5-dihydro-2-[4-methoxy-2-(1-methyle-thoxy)phenyl]-1H-imidazol-1-yl]carbonyl]-2-piperazinone or a pharmaceutically acceptable salt thereof; or
the imidazoline compound comprises a compound having the structure: 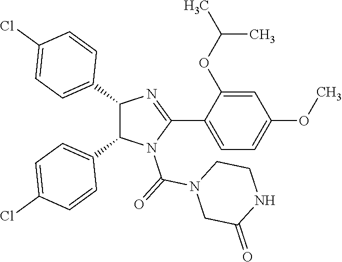 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]
or a pharmaceutically acceptable salt thereof.

38-40. (canceled)

41: The method of claim 27, wherein the administering comprises administering an amount of the senolytic agent and/or a frequency of dosage that is less than would be effective for treating cancer.

42: The method of claim 27, wherein the administering comprises a period of treatment followed by a non-treatment interval of at least two weeks.

43: The method of claim 27, wherein the administering comprises at least two treatment cycles of the senolytic agent, each treatment cycle independently including a treatment period of one day to three months followed by the non-treatment interval.

44: The method of claim 27, wherein the administering comprises a single dose of the senolytic agent followed by the non-treatment interval.

45: The method of claim 27, wherein:
said pathology comprises a pathology selected from the group consisting of a cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke), a neurodegenerative disease (e.g., Alzheimer's disease, Parkinson's disease, Huntington's disease, dementia, mild cognitive impairment, and motor neuron dysfunction), a metabolic disease (e.g., diabetes, diabetic ulcer, metabolic syndrome, and obesity), and a senescence-associated disease; or
said pathology comprises a senescence-associated disease that comprises a pulmonary disorder (e.g., pulmonary fibrosis, chronic obstructive pulmonary disease, asthma, cystic fibrosis, emphysema, bronchiectasis, and age-related loss of pulmonary function; or
said pathology comprises a senescence-associated disease that comprises an eye disease (e.g., macular degeneration, glaucoma, cataracts, presbyopia, and vision loss); or
said pathology comprises a senescence-associated disease that comprises an age-related disorder selected from the group consisting of renal disease, renal failure, frailty, hearing loss, muscle fatigue, skin conditions, skin wound healing, liver fibrosis, pancreatic fibrosis, oral submucosa fibrosis, and sarcopenia; or
said pathology comprises a senescence-associated disease that comprises a dermatological disease or disorder (e.g., eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, cutaneous lupus, etc.); or
said pathology comprises a senescence-associated disease selected from the group consisting of atherosclerosis, osteoarthritis, pulmonary fibrosis, hypertension, and chronic obstructive pulmonary disease.

46-50. (canceled)

51: The method of claim 27, wherein the senolytic agent is administered locally at or near the site of the disease or disorder.

52: The method of claim 51, wherein the senolytic agent is administered to an osteoarthritic joint.

53: The method according to any of claim 27, wherein said mammal is a human.

54: The method of claim 27, wherein said mammal is a non-human mammal.

55: The method of claim 27, wherein said method (preferentially) reduces the levels of senescent cells in said mammal.

56: A method of evaluating the efficacy of a treatment of a pathology characterized by elevated senescent cells, said method comprising:
determining a first level of one or more indicators of senescent cells in a biological sample from said mammal wherein said one or more indicators are selected from the group consisting of an eicosanoid, an eicosanoid precursor, leukotriene A4 (LTA4), leukotriene B4 (LTB4), PGD2, and 5-HETE;
treating said mammal for a pathology characterized by elevated senescent cells; and
determining a second level of said one or more indicators of senescent cells in said mammal after or during said treating, wherein a decrease in the second level of said indicator(s) as compared to the first level of said indicators indicates that said treatment is effective and the absence of change in level or an increase in the second level of said indicator(s) as compared to the first level of said indicators indicates that said treating is not effective.

57-58. (canceled)
